(19) United States
(12) Patent Application Publication
(10) Pub. No.: US 2026/0242449 A1
(43) Pub. Date: Aug. 20, 2026

(54) MULTISPECIFIC ANTI-HIV ANTIBODIES

(71) Applicant: INTERNATIONAL AIDS VACCINE INITIATIVE, New York, NY (US)

(72) Inventors: Joseph JARDINE, New York, NY (US); Mateusz KEDZIOR, New York, NY (US)

(21) Appl. No.: 19/165,009

(22) PCT Filed: Mar. 14, 2024
(86) PCT No.: PCT/US2024/019848

§ 371 Date: Sep. 12, 2025

Related U.S. Application Data
(60) Provisional application No. 63/451,968, filed on Mar. 14, 2023.

(51) Int. Cl. C07K 16/114; A61P 31/18

(52) U.S. Cl. CPC C07K 16/114; A61P 31/18; C07K 2317/31

(57) ABSTRACT

The present disclosure relates to anti-HIV Env antibodies and their use in the treatment or prevention of HIV/AIDS.

### CROSS-REFERENCE TO RELATED APPLICATIONS

This application claims priority of U.S. Provisional Application No. 63/451,968 filed Mar. 14, 2023, which is incorporated herein in its entirety.

### REFERENCE TO SEQUENCE LISTING SUBMITTED ELECTRONICALLY

The content of the electronically submitted sequence listing (Name: 6765_0806_Sequence_Listing.xml; Size: 212,905 bytes; and Date of Creation: Mar. 12, 2024) is incorporated herein by reference in its entirety.

### FIELD OF THE INVENTION

The field of the invention generally relates to anti-HIV Env antibodies and their use in the treatment or prevention of HIV/AIDS.

### BACKGROUND

While great progress has been made in the treatment of HIV/AIDS, all individuals living with HIV will have to be treated with anti-retroviral therapy (ART) for the rest of their lives since drug therapy is unable to clear latent viral reservoirs that exist in resting CD4+ T cells at a frequency of about 1/10⁶ cells. See, Eriksson, S. 2013. PLoS Pathog 9:e1003174.

Until a vaccine is discovered, many agree that a single product or approach will not completely halt new HIV infections. Accordingly, the use of HIV broadly neutralizing antibodies (bnAbs) has the potential to complement existing prevention methods by addressing important shortfalls or gaps in current product profiles. Passive immunization trials are underway in Southern Africa to test the efficacy of a single bnAb or a combination of two bnAbs in preventing new HIV infection. Clinicaltrials.gov. Safety and pharmacokinetics of the combination broadly neutralizing antibodies, 3BNC117-LS-J and 10-1074-LS-J, in healthy American and African adults. clinicaltrials.gov/ct2/show/NCT04173819 (accessed on May 21, 2021). Clinical trials involving engineered antibodies for HIV-1 therapy and cure are discussed in Grobben at al., Current Opinion in Virology, 38: 70-80 (2019). Clinicaltrials.gov/ct2/show/NCT03721510 discloses a Phase 1/2a study of PGT121, VRC07-523LS and PGDM1400 monoclonal antibodies in HIV-uninfected and HIV-infected adults.

There remains a need for the development of methods of treatment and prevention of HIV comprising the use broadly neutralizing antibodies.

### BRIEF SUMMARY

In one aspect, provided herein is a multispecific anti-HIV antibody comprising at least two antigen binding domains selected from the group consisting of (a) an antigen binding domain that binds to the V3 loop epitope region of HIV Env; (b) an antigen binding domain that binds to the V1/V2 apex epitope region of HIV Env; and (c) an antigen binding domain that binds to the CD4bs epitope region of HIV Env. In some embodiments, the multispecific antibody is a bispecific antibody. In some embodiments, the multispecific antibody is a trispecific antibody.

In some embodiments, a multispecific anti-HIV antibody described herein comprises at least two antigen binding domains selected from the group consisting of (a) an antigen binding domain that binds to the V3 loop epitope region of HIV Env and comprises a VH domain and a VL domain comprising the 3 VH CDRs and 3 VL CDRs, respectively, of ePGT121v1 or ePGT121v2; (b) an antigen binding domain that binds to the V1/V2 apex epitope region of HIV Env and comprises a VH domain and a VL domain comprising the 3 VH CDRs and 3 VL CDRs, respectively, of ePGDM1400v9; and (c) an antigen binding domain that binds to the CD4bs epitope region of HIV Env and comprises a VH domain and a VL domain comprising the 3 VH CDRs and 3 VL CDRs, respectively, of N49P7. In some embodiments, the multispecific antibody is a bispecific antibody. In some embodiments, the multispecific antibody is a trispecific antibody.

In some embodiments, a bispecific antibody described herein comprises an scFv domain linked to a first Fc domain and an Fab domain linked to a second Fc domain, wherein the (i) scFv domain linked to the first Fc domain, (ii) Fab heavy chain linked to the second Fc domain and (iii) Fab light chain comprise an amino acid sequence having at least 80%, at least 85%, at least 90%, at least 95%, at least 97% or 100% identity to SEQ ID NO: 13, 56, and 60, respectively; SEQ ID NO: 14, 56, and 60, respectively; SEQ ID NO: 13, 57, and 60, respectively; SEQ ID NO: 14, 57, and 60, respectively; SEQ ID NO: 41, 56, and 60, respectively; SEQ ID NO: 42, 56, and 60, respectively; SEQ ID NO: 41, 57, and 60, respectively; or SEQ ID NO: 42, 57, and 60, respectively.

In some embodiments, a bispecific antibody described herein comprises a first scFv domain linked to a first Fc domain and a second scFv domain linked to a second Fc domain, wherein the (i) first scFv domain linked to the first Fc domain, (ii) and second scFv domain linked to the second Fc domain comprise an amino acid sequence having at least 80%, at least 85%, at least 90%, at least 95%, at least 97% or 100% identity to SEQ ID NO: 13 and 44, respectively; SEQ ID NO: 14 and 44, respectively; SEQ ID NO: 13 and 43, respectively; or SEQ ID NO: 14 and 43, respectively.

In some embodiments, a trispecific antibody described herein comprises a first scFv linked to a first Fc domain, an antibody heavy chain comprising a second Fc domain, and second Fc domain linked to an antibody light chain, wherein the first scFv linked to a first Fc domain, the antibody heavy chain comprising a second Fc domain, and the second Fc domain linked to an antibody light chain comprise an amino acid sequence having at least 80%, at least 85%, at least 90%, at least 95%, at least 97% or 100% identity to SEQ ID NO: 41, 16 and 142, respectively, or 42, 16 and 142, respectively.

In some embodiments, a trispecific antibody described herein comprises a first and second scFv linked to a first Fc domain, an antibody heavy chain comprising a second Fc domain, and an antibody light chain, wherein the first and second scFv linked to a first Fc domain, the antibody heavy chain comprising a second Fc domain, and the antibody light chain comprise an amino acid sequence having at least 80%, at least 85%, at least 90%, at least 95%, at least 97% or 100% identity to SEQ ID NO: 65, 57 and 60, respectively, or 66, 57 and 60, respectively.

In some embodiments, a trispecific antibody described herein comprises a first scFv linked to a first Fc domain, second Fc domain linked to an antibody heavy chain comprising a second Fc domain, and an antibody light chain, wherein the first scFv linked to a first Fc domain, the second Fc domain linked to an antibody heavy chain comprising a second Fc domain, and the antibody light chain comprise an amino acid sequence having at least 80%, at least 85%, at least 90%, at least 95%, at least 97% or 100% identity to SEQ ID NO: 13, 67 and 60, respectively, or 13, 68 and 60, respectively.

In some embodiments, a bispecific antibody described herein is BiS1, BiS 2, BiS 3, BiS 4, BiS 5, BiS 6, BiS 7, BiS 8. BiS 9, BiS 10, BiS 11, BiS 12, BiS 13 or BiS 14. In some embodiments, a trispecific antibody described herein is TriS 1, TriS 2, TriS 3, TriS 4, TriS 5 or TriS 6.

In one aspect, provided herein is an isolated polynucleotide encoding a multispecific antibody described herein. In some embodiments, the isolated polynucleotide comprises a nucleotide sequence encoding a signal peptide. In some embodiments, a polynucleotide encoding a multispecific antibody described herein is DNA. In some embodiments, a polynucleotide described herein encoding a multispecific antibody described herein is RNA. In some embodiments, the polynucleotide is an mRNA comprising a modified ribonucleotide.

In one aspect, provided herein is a vector comprising a polynucleotide encoding a multispecific antibody described herein. In some embodiments, the vector comprises more than one isolated polynucleotide. In some embodiments, a vector comprising a polynucleotide encoding a multispecific antibody described herein comprises 2 isolated polynucleotides. In some embodiments, a vector comprising a polynucleotide encoding a multispecific antibody described herein comprises 3 isolated polynucleotides. In some embodiments, a vector comprising a polynucleotide encoding a multispecific antibody described herein comprises more than one vector. In some embodiments, a vector comprising a polynucleotide encoding a multispecific antibody described herein comprises 2 vectors. In some embodiments, a vector comprising a polynucleotide encoding a multispecific antibody described herein comprises 3 vectors.

In some embodiments, a polynucleotide encoding a bispecific antibody described herein comprises 3 polynucleotides comprising the nucleotide sequence of SEQ ID NO: 82-84. In some embodiments, a polynucleotide encoding a bispecific antibody described herein comprises 3 polynucleotides comprising the nucleotide sequence of SEQ ID NO: 85-87. In some embodiments, a polynucleotide encoding a bispecific antibody described herein comprises 3 polynucleotides comprising the nucleotide sequence of SEQ ID NO: 88-90. In some embodiments, a polynucleotide encoding a bispecific antibody described herein comprises 3 polynucleotides comprising the nucleotide sequence of SEQ ID NO: 91-93. In some embodiments, a polynucleotide encoding a bispecific antibody described herein comprises 3 polynucleotides comprising the nucleotide sequence of SEQ ID NO: 94-96. In some embodiments, a polynucleotide encoding a bispecific antibody described herein comprises 3 polynucleotides comprising the nucleotide sequence of SEQ ID NO: 97-99. In some embodiments, a polynucleotide encoding a bispecific antibody described herein comprises 3 polynucleotides comprising the nucleotide sequence of SEQ ID NO: 100-102. In some embodiments, a polynucleotide encoding a bispecific antibody described herein comprises 3 polynucleotides comprising the nucleotide sequence of SEQ ID NO: 103-105. In some embodiments, a polynucleotide encoding a bispecific antibody described herein comprises 2 polynucleotides comprising the nucleotide sequence of SEQ ID NO: 106 and 107. In some embodiments, a polynucleotide encoding a bispecific antibody described herein comprises 2 polynucleotides comprising the nucleotide sequence of SEQ ID NO: 108 and 109. In some embodiments, a polynucleotide encoding a bispecific antibody described herein comprises 2 polynucleotides comprising the nucleotide sequence of SEQ ID NO: 110 and 111. In some embodiments, a polynucleotide encoding a bispecific antibody described herein comprises 2 polynucleotides comprising the nucleotide sequence of SEQ ID NO: 112 and 113. In some embodiments, a polynucleotide encoding a bispecific antibody described herein comprises 2 polynucleotides comprising the nucleotide sequence of SEQ ID NO: 114 and 115. In some embodiments, a polynucleotide encoding a bispecific antibody described herein comprises 2 polynucleotides comprising the nucleotide sequence of SEQ ID NO: 116 and 117. In some embodiments, the polynucleotides encoding a bispecific antibody described herein further comprise a nucleotide sequence encoding a signal peptide. In some embodiments, the signal peptide comprises the amino acid sequence of SEQ ID NO: 69 or 70. In some embodiments, the nucleotide sequence encoding a signal peptide comprises SEQ ID NO: 136 or 137.

In some embodiments, a polynucleotide encoding a trispecific antibody described herein comprises 3 polynucleotides comprising the nucleotide sequence of SEQ ID NO: 118-120. In some embodiments, a polynucleotide encoding a trispecific antibody described herein comprises 3 polynucleotides comprising the nucleotide sequence of SEQ ID NO: 121-123. In some embodiments, a polynucleotide encoding a trispecific antibody described herein comprises 3 polynucleotides comprising the nucleotide sequence of SEQ ID NO: 124-126. In some embodiments, a polynucleotide encoding a trispecific antibody described herein comprises 3 polynucleotides comprising the nucleotide sequence of SEQ ID NO: 127-129. In some embodiments, a polynucleotide encoding a trispecific antibody described herein comprises 3 polynucleotides comprising the nucleotide sequence of SEQ ID NO: 130-132. In some embodiments, a polynucleotide encoding a trispecific antibody described herein comprises 3 polynucleotides comprising the nucleotide sequence of SEQ ID NO: 133-135. In some embodiments, the polynucleotides encoding a bispecific antibody described herein further comprise a nucleotide sequence encoding a signal peptide. In some embodiments, the signal peptide comprises the amino acid sequence of SEQ ID NO: 69 or 70. In some embodiments, the nucleotide sequence encoding a signal peptide comprises SEQ ID NO: 136 or 137.

In one aspect, provided herein is a host cell capable of producing the multispecific antibody described herein. In some embodiments, the host cell is a CHO cell or a HEK293 cell.

In one aspect, provided herein is a host cell comprising a polynucleotide described herein or a vector described herein. In some embodiments, the host cell is a CHO cell or a HEK293 cell.

In one aspect, provided herein is a recombinant virus comprising a polynucleotide encoding a multispecific antibody described herein.

In one aspect, provided herein is a pharmaceutical composition comprising a multispecific antibody described herein and a pharmaceutically acceptable excipient.

In one aspect, provided herein is a pharmaceutical composition comprising an isolated polynucleotide described herein and a pharmaceutically acceptable excipient. In some embodiments, the polynucleotide is RNA. In some embodiments, the RNA is mRNA comprising a modified ribonucleotide.

In one aspect, provided herein is a method of neutralizing an HIV virus comprising contacting the virus with a sufficient amount of a multispecific antibody described herein or a pharmaceutical composition described herein.

In one aspect, provided herein is a method of reducing the likelihood of HIV infection in a subject exposed to HIV comprising administering to the subject a therapeutically sufficient amount of a multispecific antibody described herein or a pharmaceutical composition described herein. In some embodiments, the method further comprises administering at least one additional therapeutic agent. In some embodiments, the additional therapeutic agent is an antiretroviral agent, a reservoir activator, or an additional antibody. In some embodiments, the additional therapeutic agent comprises an additional antibody, which is broadly neutralizing antibody.

In one aspect, provided herein is a method of treating HIV/AIDS comprising administering to a subject in need thereof a therapeutically sufficient amount of a multispecific antibody described herein or a pharmaceutical composition described herein. In some embodiments, the method further comprises administering at least one additional therapeutic agent. In some embodiments, the additional therapeutic agent is an antiretroviral agent, a reservoir activator, or an additional antibody. In some embodiments, the additional therapeutic agent comprises an additional antibody, which is broadly neutralizing antibody.

In one aspect, provided herein is a method of reducing viral load comprising administering to a subject in need thereof a therapeutically sufficient amount of a multispecific antibody described herein or a pharmaceutical composition described herein. In some embodiments, the method further comprises administering at least one additional therapeutic agent. In some embodiments, the additional therapeutic agent is an antiretroviral agent, a reservoir activator, or an additional antibody. In some embodiments, the additional therapeutic agent comprises an additional antibody, which is broadly neutralizing antibody.

In some embodiments, the method further comprises administering at least one additional therapeutic agent. In some embodiments, the additional therapeutic agent is an antiretroviral agent, a reservoir activator, or an additional antibody. In some embodiments, the additional therapeutic agent comprises an additional antibody, which is broadly neutralizing antibody. In some embodiments, the method further comprises administering at least one additional therapeutic agent. In some embodiments, the additional therapeutic agent is an antiretroviral agent, a reservoir activator, or an additional antibody. In some embodiments, the additional therapeutic agent comprises an additional antibody, which is broadly neutralizing antibody.

In some embodiments, the disclosure provides:
[1.] A multispecific anti-HIV antibody comprising at least two antigen binding domains selected from the group consisting of
(a) an antigen binding domain that binds to the V3 loop epitope region of HIV Env;
(b) an antigen binding domain that binds to the V1/V2 apex epitope region of HIV Env; and
(c) an antigen binding domain that binds to the CD4bs epitope region of HIV Env;
[2.] the multispecific antibody of [1], wherein the antigen binding domain that binds to the V3 loop epitope region of HIV Env comprises a VH domain and a VL domain comprising the 3 VH CDRs and 3 VL CDRs, respectively, of PGT121 or an engineered variant of PGT121;
[3.] the multispecific antibody of [2], wherein the VH domain comprises the VH CDR1, VH CDR2, VH CDR3 of ePGT121v1 or ePGT121v2, and the VL domain comprises the VL CDR1, VL CDR2 and VL CDR3 of ePGT121v1 or ePGT121v2;
[4.] the multispecific antibody of [3], wherein the VH CDR1, VH CDR2, VH CDR3, VL CDR1, VL CDR2 and VL CDR3 comprise the amino acid sequence of SEQ ID NO: 1, 2, 3, 5, 6, and 7, respectively, or SEQ ID NO: 19, 20, 21, 23, 24 and 25, respectively;
[5.] the multispecific antibody of any one of [2] to [4], wherein the VH domain and VL domain comprises an amino acid sequence having at least 80%, at least 85%, at least 90%, at least 95%, at least 97% or 100% identity to SEQ ID NO: 4 and 8, respectively, or 22 and 26, respectively;
[6.] the multispecific antibody of any one of [2] to [5], wherein the antigen binding domain comprises an scFv domain or an Fab domain;
[7.] the multispecific antibody of [6], wherein the antigen binding domain comprises an scFv comprising the amino acid sequence of SEQ ID NO: 9, 10, 11 or 12;
[8.] the multispecific antibody of [8], wherein the antigen binding domain comprises an Fab domain comprising a heavy chain and light chain having the amino acid sequence of SEQ ID NO: 17 and 18, respectively, or 28 and 29, respectively;
[9.] the multispecific antibody of [1], wherein the antigen binding domain that binds to the V1/V2 apex epitope region of HIV Env comprises a VH domain and a VL domain comprising the 3 VH CDRs and 3 VL CDRs, respectively, of PGDM1400 or an engineered variant of PGDM1400;
[10.] the multispecific antibody of [9], wherein the VH domain comprises the VH CDR1, VH CDR2, VH CDR3 of ePGDM1400v9, and the VL domain comprises the VL CDR1, VL CDR2 and VL CDR3 of ePGDM1400v9;
[11.] the multispecific antibody of [10], wherein the VH CDR1, VH CDR2, VH CDR3, VL CDR1, VL CDR2 and VL CDR3 comprise the amino acid sequence of SEQ ID NO: 30, 31, 32, 34, 35 and 36, respectively;
[12.] the multispecific antibody of any one of [9] to [11], wherein the VH domain and VL domain comprises an amino acid sequence having at least 80%, at least 85%, at least 90%, at least 95%, at least 97% or 100% identity to SEQ ID NO: 33 and 38, respectively;
[13.] the multispecific antibody of any one of [9] to [12], wherein the antigen binding domain comprises an scFv domain or an Fab domain;
[14.] the multispecific antibody of [13], wherein the antigen binding domain comprises an scFv comprising the amino acid sequence of SEQ ID NO: 39 or 40;
[15.] the multispecific antibody of [13], wherein the antigen binding domain comprises an Fab domain comprising a VH and VL having the amino acid sequence of SEQ ID NO: 33 and 38, respectively;
[16.] the multispecific antibody of [1], wherein the antigen binding domain that binds to the CD4bs epitope region of HIV Env comprises a VH domain and a VL domain comprising the 3 VH CDRs and 3 VL CDRs, respectively, of N49P7 or an engineered variant of N49P7;
[17.] the multispecific antibody of [16], wherein the VH domain comprises the VH CDR1, VH CDR2, VH CDR3 of N49P7, and the VL domain comprises the VL CDR1, VL CDR2 and VL CDR3 of N49P7;
[18.] the multispecific antibody of [17], wherein the VH CDR1, VH CDR2, VH CDR3. VL CDR1, VL CDR2 and VL CDR3 comprise the amino acid sequence of SEQ ID NO: 45, 46, 47, 50, 51 and 52, respectively;
[19.] the multispecific antibody of any one of [16] to [18], wherein the VH domain and VL domain comprises an amino acid sequence having at least 80%, at least 85%, at least 90%, at least 95%, at least 97% or 100% identity to SEQ ID NO: 48 and 53, respectively, or 49 and 53, respectively;
[20.] the multispecific antibody of any one of [16] to [19], wherein the antigen binding domain comprises an scFv domain or an Fab domain;
[21.] the multispecific antibody of [20], wherein the antigen binding domain comprises an scFv comprising the amino acid sequence of SEQ ID NO: 54 or 55;
[22.] the multispecific antibody of [20], wherein the antigen binding domain comprises an Fab domain comprising a heavy chain and light chain having the amino acid sequence of SEQ ID NO: 58 and 60, respectively, or 59 and 60, respectively;
[23.] the multispecific antibody of any one of [1] to [22], which is a bispecific antibody;
[24.] the multispecific antibody of [23] comprising an scFv domain linked to an antibody, wherein the scFv domain and the antibody have different binding specificities;
[25.] the multispecific antibody of [24], wherein the C-terminal end of the scFv domain is linked to the N-terminal end of the antibody light chain;
[26.] the multispecific antibody of [25], wherein the scFv linked to the antibody light chain and the antibody heavy chain comprises an amino acid sequence having at least 80%, at least 85%, at least 90%, at least 95%, at least 97% or 100% identity to SEQ ID NO: 61 and 27, respectively, or 62 and 27, respectively;
[27.] the multispecific antibody of [23] comprising
(a) an scFv domain linked to a first Fc domain and an Fab domain linked to a second Fc domain, wherein the scFv and Fab domains have different binding specificities; or
(b) a first scFv domain linked to a first Fc domain and a second scFv domain linked to a second Fc domain, wherein the first and second scFv domains have different binding specificities;
[28.] the multispecific antibody of [27], wherein the bispecific antibody comprises an scFv domain linked to a first Fc domain and an Fab domain linked to a second Fc domain, wherein the
(i) scFv domain, (ii) Fab heavy chain and (iii) Fab light chain comprise an amino acid sequence having at least 80%, at least 85%, at least 90%, at least 95%, at least 97% or 100% identity to
(a) SEQ ID NO: 9, 58, and 60, respectively;
(b) SEQ ID NO: 10, 58, and 60, respectively;
(c) SEQ ID NO: 11, 58, and 60, respectively;
(d) SEQ ID NO: 12, 58, and 60, respectively;
(e) SEQ ID NO: 9, 59, and 60, respectively;
(f) SEQ ID NO: 10, 59, and 60, respectively;
(g) SEQ ID NO: 11, 59, and 60, respectively;
(h) SEQ ID NO: 12, 59, and 60, respectively;
(i) SEQ ID NO: 39, 58, and 60, respectively;
(j) SEQ ID NO: 40, 58, and 60, respectively;
(k) SEQ ID NO: 39, 59, and 60, respectively; or
(l) SEQ ID NO: 40, 59, and 60, respectively;
[29.] the multispecific antibody of [27], wherein the bispecific antibody comprises a first scFv domain linked to a first Fc domain and a second scFv domain linked to a second Fc domain, wherein the (i) first scFv domain, (ii) and second scFv domain comprise an amino acid sequence having at least 80%, at least 85%, at least 90%, at least 95%, at least 97% or 100% identity to
(a) SEQ ID NO: 9 and 40, respectively;
(b) SEQ ID NO: 10 and 40, respectively;
(c) SEQ ID NO: 11 and 40, respectively;
(d) SEQ ID NO: 12 and 40, respectively;
(e) SEQ ID NO: 9 and 39, respectively;
(f) SEQ ID NO: 10 and 39, respectively;
(g) SEQ ID NO: 11 and 39, respectively; or
(h) SEQ ID NO: 12 and 39, respectively;
[30.] the multispecific antibody of any one of [27] to [29], wherein the first and second Fc domains are IgG Fc domains, and wherein
(a) the first Fc domain comprises the T366W substitution and the second Fc domain comprises the T366S, L368A and Y407V substitutions;
(b) the first Fc domain comprises the T366S, L368A and Y407V substitutions and the second Fc domain comprises the T366W substitution;
[31.] the multispecific antibody of [30], wherein the bispecific antibody comprises an scFv domain linked to a first Fc domain and an Fab domain linked to a second Fc domain, wherein the (i) scFv domain linked to the first Fc domain, (ii) Fab heavy chain linked to the second Fc domain and (iii) Fab light chain comprise an amino acid sequence having at least 80%, at least 85%, at least 90%, at least 95%, at least 97% or 100% identity to
(a) SEQ ID NO: 13, 56, and 60, respectively;
(b) SEQ ID NO: 14, 56, and 60, respectively;
(c) SEQ ID NO: 13, 57, and 60, respectively;
(d) SEQ ID NO: 14, 57, and 60, respectively;
(e) SEQ ID NO: 41, 56, and 60, respectively;
(f) SEQ ID NO: 42, 56, and 60, respectively;
(g) SEQ ID NO: 41, 57, and 60, respectively; or
(h) SEQ ID NO: 42, 57, and 60, respectively;
[32.] the multispecific antibody of [30], wherein the bispecific antibody comprises a first scFv domain linked to a first Fc domain and a second scFv domain linked to a second Fc domain, wherein the (i) first scFv domain linked to the first Fc domain, (ii) and second scFv domain linked to the second Fc domain comprise an amino acid sequence having at least 80%, at least 85%, at least 90%, at least 95%, at least 97% or 100% identity to
(a) SEQ ID NO: 13 and 44, respectively;
(b) SEQ ID NO: 14 and 44, respectively;
(c) SEQ ID NO: 13 and 43, respectively; or
(d) SEQ ID NO: 14 and 43, respectively;
[33.] the multispecific antibody of any one of [1] to [22], which is a trispecific antibody;
[34.] the multispecific antibody of [33], wherein the trispecific antibody comprises
(a) a first scFv linked to a first Fc domain, an antibody heavy chain comprising a second Fc domain, and second Fc domain linked to an antibody light chain;
(b) a first and second scFv linked to a first Fc domain, an antibody heavy chain comprising a second Fc domain, and an antibody light chain; or
(c) a first scFv linked to a first Fc domain, second Fc domain linked to an antibody heavy chain comprising a second Fc domain, and an antibody light chain;
[35.] the multispecific antibody of [34], wherein the first and second Fc domains are IgG Fc domains, and wherein
(a) the first Fc domain comprises the T366W substitution and the second Fc domain comprises the T366S, L368A and Y407V substitutions;
(b) the first Fc domain comprises the T366S, L368A and Y407V substitutions and the second Fc domain comprises the T366W substitution;
[36.] the multispecific antibody of [34] or [35], wherein
(a) the first scFv linked to a first Fc domain, the antibody heavy chain comprising a second Fc domain, and the second Fc domain linked to an antibody light chain comprise an amino acid sequence having at least 80%, at least 85%, at least 90%, at least 95%, at least 97% or 100% identity to SEQ ID NO: 41, 16 and 142, respectively, or 42, 16 and 142, respectively;
(b) the first and second scFv linked to a first Fc domain, the antibody heavy chain comprising a second Fc domain, and the antibody light chain comprise an amino acid sequence having at least 80%, at least 85%, at least 90%, at least 95%, at least 97% or 100% identity to SEQ ID NO: 65, 57 and 60, respectively, or 66, 57 and 60, respectively; or
(c) the first scFv linked to a first Fc domain, the second Fc domain linked to an antibody heavy chain comprising a second Fc domain, and the antibody light chain comprise an amino acid sequence having at least 80%, at least 85%, at least 90%, at least 95%, at least 97% or 100% identity to SEQ ID NO: 13, 67 and 60, respectively, or 13, 68 and 60, respectively.
[37.] An isolated polynucleotide encoding the multispecific antibody of any one of [1] to [36];
[38.] the isolated polynucleotide of [37], wherein the polynucleotide comprises a nucleotide sequence encoding a signal peptide;
[39.] the isolated polynucleotide of [38], wherein the signal peptide comprises the amino acid sequence of SEQ ID NO: 69 or 70;
[40.] the polynucleotide of any one of [37] to [39] that is DNA;
[41.] the polynucleotide of any one of [37] to [39] that is RNA;
[42.] the polynucleotide of [41], wherein the RNA is mRNA comprising a modified ribonucleotide.
[43.] A vector comprising the polynucleotide of any one of [37] to [39];
[44.] the vector of [43] comprising more than one isolated polynucleotide.
[45.] A host cell capable of producing the multispecific antibody of any one of [1] to [36].
[46.] A host cell comprising the polynucleotide of any one of [37] to [39] or the vector of [43] or [44];
[47.] the host cell of [46] which is a CHO cell or a HEK293 cell.
[48.] A recombinant virus comprising the polynucleotide of any one of [37] to [39].
[49.] A pharmaceutical composition comprising the multispecific antibody of any one of [1] to [36] and a pharmaceutically acceptable excipient.
[50.] A pharmaceutical composition comprising the isolated polynucleotide of any one of [37] to [42] and a pharmaceutically acceptable excipient;
[51.] the pharmaceutical composition of [50], wherein the polynucleotide is RNA;
[52.] the pharmaceutical composition of [51], wherein the RNA is mRNA comprising a modified ribonucleotide.
[53.] A method of neutralizing an HIV virus comprising contacting the virus with a sufficient amount of a multispecific antibody of any one of [1] to [36] or a pharmaceutical composition according to any one of [49] to [52].
[54.] A method of reducing the likelihood of HIV infection in a subject exposed to HIV comprising administering to the subject a therapeutically sufficient amount of a multispecific antibody of any one of [1] to [36] or a pharmaceutical composition according to any one of [49] to [52].
[55.] A method of treating HIV/AIDS comprising administering to a subject in need thereof a therapeutically sufficient amount of a multispecific antibody of any one of [1] to [36] or a pharmaceutical composition according to any one of [49] to [52].
[56.] A method of reducing viral load comprising administering to a subject in need thereof a therapeutically sufficient amount of a multispecific antibody of any one of [1] to [36] or a pharmaceutical composition according to any one of [49] to [52];
[57.] the method of any one of [54] to [56] further comprising administering at least one additional therapeutic agent;
[58.] the method of [57], wherein the additional therapeutic agent is an antiretroviral agent, a reservoir activator, or an additional antibody;
[59.] the method of [58], wherein the additional therapeutic agent comprises an additional antibody, which is broadly neutralizing antibody.

### BRIEF DESCRIPTION OF THE DRAWINGS

FIG. 1. Examples of bispecific antibody compositions.

FIG. 2. Examples of trispecific antibody compositions.

FIG. 3. Pseudovirus neutralization by BiS 1-12 and TriS 1-2 using a panel of 10 cross-clade isolates.

FIG. 4. Pseudovirus neutralization by BiS 1-12 and TriS 1-2. IC80 against the panel of 10 cross-clade isolates shown in FIG. 3.

FIG. 5. Pseudovirus neutralization by TriS 1 and TriS 2 using a panel of 34 cross-clade isolates.

FIG. 6. Pseudovirus neutralization by TriS 1 and TriS 2. IC80 against the panel of 34 cross-clade isolates shown in FIG. 5.

FIG. 7. Pseudovirus neutralization by BiS 13 using a panel of 40 cross-clade isolates.

FIG. 8. Pseudovirus neutralization by BiS 13. A) IC80 against the panel of 40 cross-clade isolates shown in FIG. 7. B) IC80 against Q259.17, MS208.A1, 6545.V4.C1, BJOX009000.02.4, CNE8, BJIC80, T257-31, QH0692.42-N332A, TRO.11-N332A, CAP45.G3, 3016.v5.C45, 231965.c01 and X1632.S2.B10 isolates.

FIG. 9. Pseudovirus neutralization by BiS 14 using a panel of 12 cross-clade isolates.

FIG. 10. Pseudovirus neutralization by BiS 14. IC80 against the panel of 12 cross-clade isolates shown in FIG. 7.

### DETAILED DESCRIPTION

### I. Definitions

To facilitate an understanding of the present invention, a number of terms and phrases are defined below.

The terms “human immunodeficiency virus” or “HIV,” as used herein, refer generally to a retrovirus that is the causative agent for acquired immunodeficiency syndrome (AIDS), variants thereof (e.g., simian acquired immunodeficiency syndrome, SAIDS), and diseases, conditions, or opportunistic infections associated with AIDS or its variants, and includes HIV-Type 1 (HIV-1) and HIV-Type 2 (HIV-2) of any clade or strain therein, related retroviruses (e.g., simian immunodeficiency virus (SIV)), and variants thereof (e.g., engineered retroviruses, e.g., chimeric HIV viruses, e.g., simian-human immunodeficiency viruses (SHIVs)). In some embodiments, an HIV virus is an HIV-Type-1 virus. Previous names for HIV include human T-lymphotropic virus-Ill (HTLV-III), lymphadenopathy-associated virus (LAV), and AIDS-associated retrovirus (ARV).

As used herein, the term “clade” refers to related human immunodeficiency viruses (HIVs) classified according to their degree of genetic similarity. There are currently four known groups of HIV-1 isolates: M, N, O, and P. Group M (major strains) viruses are responsible for the majority of the global HIV epidemic. The other three groups, i.e., N, O and P are quite uncommon and only occur in Cameroon, Gabon and Equatorial Guinea. In some embodiments, an HIV virus is a Group M HIV virus. Within group M there are known to be at least nine genetically distinct subtypes or clades of HIV-1: subtypes or clades A, B, C, D, F, G, H, J and K. Additionally, different subtypes can combine genetic material to form a hybrid virus, known as a ‘circulating recombinant form’ (CRFs). Subtype/clade B is the dominant HIV subtype in the Americas, Western Europe and Australasia. Subtype/clade C is very common in the high AIDS prevalence countries of Southern Africa, as well as in the horn of Africa and India. Just under half of all people living with HIV have subtype C. In certain exemplary embodiments, methods described herein can be used to treat a subject (e.g., a human) infected with HIV (e.g., HIV-1) or to block or prevent HIV (e.g., HIV-1) infection in subject (e.g., a human) at risk of HIV transmission. The HIV may be of two, three, four, five, six, seven, eight, nine, ten, or more clades and/or two or more groups of HIV.

Acquired immune deficiency syndrome (“AIDS”) is a disease caused by the human immunodeficiency virus, or HIV.

As used herein, the term “envelope glycoprotein” or “Env” refers to the glycoprotein that is expressed on the surface of the envelope of HIV virions and the surface of the plasma membrane of HIV infected cells. “Envelope glycoprotein” or “Env” encompass, but are not limited to, native Env, an isoform of Env, or a variant of Env (e.g., SOSIP) derived from an HIV isolate, for example, BG505. Env is the sole virally encoded gene product on the surface of the virus and, as such, is the only target of neutralizing antibodies. Env is a trimer of heterodimers composed of two non-covalently associated subunits: the receptor-binding gp120 and the fusion machinery-containing gp41. Each subunit is derived from a gp160 precursor glycoprotein following cleavage by cellular furins. HIV-1 gp120 binds the CD4 molecule on the surface of human target T cells to initiate the viral entry process, and following co-receptor engagement, fusion is mediated by gp41. gp140 env is the uncleaved ectodomain of gp160. In some embodiments, Env is a BG505 Env polypeptide. UniProtKB accession number Q2N0S5-1, Q2N0S6-1, and Q2N0S7-1 provide BG505 env gp160 polypeptide sequences.

HIV Env “epitope region” means a region of HIV Env defined by the overlapping epitopes recognized by particular classes of broadly neutralizing anti-HIV antibodies. See, e.g., Zhang et al., Int J Mol Sci. 17(11): 1901 (2016); Liu et al., Emerg Microbes Infect 9(1):194-206 (2020). In recent years, new techniques allowed the identification of a large number of broadly neutralizing antibodies (bNAbs) from HIV-1-infected donors. Based on their epitope specificity, the bNAbs were classified into five major group. bNAbs in each of the 5 groups recognize overlapping epitopes, which collectively define 5 epitope regions on the surface of HIV Env. These regions are the CD4 binding site (CD4bs) epitope region, V1/V2 apex epitope region, V3 loop epitope region involving Asn332 glycan, gp120-gp4l interface epitope region, and the gp41 membrane proximal external epitope region (MPER). Representative antibodies capable of binding to the CD4 binding site (CD4bs) epitope region include N49P7, VRC07, NIH45-46, 3BNC117, VRC07-523, b12, VRC01, VRC02, NIH-45-46, 3BNC60, 3BNC62, 3BNC95, 3BNC176, 12A12, VRC-PG04, VRC-CH30, VRC-CH31, VRC-CH32, VRC-CH33, VRC-CH34, VRC03, 3BNC55, 3BNC91, 3BNC104, 3BNC89, 12A21, VRC-PG04b, VRC03HC-VRC01LC, VRC01HC/VRC03LC, gVRC-H5(d74)/VRC-PG04LC, and gVRCOH12(D74)/VRC-PG04LC. Zhang et al., Int J Mol Sci. 17(11): 1901 (2016); U.S. Appl. No. 20160213779 and 20210079070, U.S. Pat. Nos. 9,175,070, 8,637,036, 9,738,703, 10,035,845, 9,695,230, and 10,035,844, and Int'l. Pat. Appl. Pub. No. WO2019165122, each of which is incorporated herein by reference for all purposes each of which is incorporated herein by reference for all purposes. Representative antibodies capable of binding to the V1/V2 apex epitope region include PG9, PG16, CH01-04, PGT141-145, PGDM1400. Representative antibodies capable of binding to the V3 loop epitope region include PGT121-123, PGT125-131, PGT135, 10-1074 and 2G12.

The term “antibody” means an immunoglobulin molecule (or a group of immunoglobulin molecules) that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the terms “antibody” and “antibodies” are terms of art and can be used interchangeably herein and refer to a molecule with an antigen-binding site that specifically binds an antigen.

Antibodies can include, for example, monoclonal antibodies, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), affybodies, Fab fragments, F(ab′)₂ fragments, disulfide-linked Fvs (sdFv), anti-idiotypic (anti-Id) antibodies (including, e.g., anti-anti-Id antibodies), bispecific antibodies, and multi-specific antibodies. In certain embodiments, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁, or IgA₂), or any subclasses (isotypes) thereof (e.g. IgG1, IgG2, IgG3, IgG4, IgA1 and IgA2), of immunoglobulin molecule, based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated or fused to other molecules such as toxins, radioisotopes, other polypeptides etc.

As used herein, the terms “antigen-binding domain,” “antigen-binding region,” “antigen-binding site,” and similar terms refer to the portion of antibody molecules which comprises the amino acid residues that confer on the antibody molecule its specificity for the antigen (e.g., HIV Env). The antigen-binding region can be derived from any animal species, such as mouse and humans.

As used herein, the terms “variable region” or “variable domain” are used interchangeably and are common in the art. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen (e.g., HIV Env). In certain embodiments, the variable region comprises 3 CDRs (CDR1, CDR2, and CDR3) and 4 framework regions (FR1, FR2, FR3, and FR4) in the order of FR1-CDR1-FR2-CDR2-FR3-CDR3-FR4 from the N terminus to the C terminus. In certain embodiments, the variable region is a human variable region. In certain embodiments, the variable region comprises human CDRs and human framework regions (FRs). In certain embodiments, the variable region comprises CDRs and framework regions (FRs) wherein one or more of the CDRs were modified by a substitution, deletion, or insertion relative to the CDRs of a parental antibody. In certain embodiments, the variable region comprises CDRs and framework regions (FRs) wherein one or more of the FRs were modified by a substitution, deletion, or insertion relative to the FRs of a parental antibody. In certain embodiments, the variable region comprises CDRs and framework regions (FRs) wherein one or more of the CDRs and one or more of the FRs were modified by a substitution, deletion, or insertion relative to the CDRs and FRs of a parental antibody. In certain embodiments, the parental antibody is PGDM1400. In certain embodiments, the variable region comprises human CDRs and primate (e.g., non-human primate) framework regions (FRs).

A skilled artisan understands that there are at several techniques for determining CDRs. One approach is based on cross-species sequence variability (i.e., Kabat et al., Sequences of Proteins of Immunological Interest. (5th ed., 1991, National Institutes of Health, Bethesda Md.) (“Kabat”). Another approach is based on crystallographic studies of antigen-antibody complexes (Al-lazikani et al, J. Molec. Biol. 273:927-948 (1997)) (“Chothia”). In addition, combinations of these two approaches are sometimes used in the art to determine CDRs. In some embodiments, the CDR sequences are identified according to Kabat. In some embodiments, the CDR sequences are identified according to Chothia. It is understood that the identification of CDRs in a variable region also identifies the FRs as the sequences flanking the CDRs.

The Kabat numbering system is generally used when referring to a residue in the variable domain (approximately residues 1-107 of the light chain and residues 1-113 of the heavy chain) (e.g., Kabat et al., Sequences of Immunological Interest. (5th Ed., 1991, National Institutes of Health, Bethesda, Md.) (“Kabat”).

The amino acid position numbering as in Kabat, refers to the numbering system used for heavy chain variable domains or light chain variable domains of the compilation of antibodies in Kabat et al. (Sequences of Immunological Interest. (5th Ed., 1991, National Institutes of Health, Bethesda, Md.), “Kabat”). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. In some embodiments, the CDR sequences are identified according to Kabat. In some embodiments, the CDR sequences are identified according to Chotia. In some embodiments, the CDR sequences are identified according to AbM. In some embodiments, the VH CDR3 sequence is identified according to Kabat. In some embodiments, the VH CDR3 sequence is identified according to Chotia. In some embodiments, the VH CDR3 sequence is identified according to AbM.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The terms “VL” and “VL domain” are used interchangeably to refer to the light chain variable region of an antibody.

The terms “VH” and “VH domain” are used interchangeably to refer to the heavy chain variable region of an antibody.

The term “antibody fragment” refers to a portion of an intact antibody. An “antigen-binding fragment” refers to a portion of an intact antibody that binds to an antigen. An antigen-binding fragment can contain the antigenic determining variable regions of an intact antibody. Examples of antibody fragments include, but are not limited to Fab, Fab′, F(ab′)2, and Fv fragments, linear antibodies, and single chain antibodies.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

The term “polyclonal antibody” describes a composition of different (diverse) antibody molecules which are capable of binding to or reacting with several different specific antigenic determinants on the same or on different antigens. Usually, the variability of a polyclonal antibody is located in the so-called variable regions of the polyclonal antibody, in particular in the CDR regions. In the present disclosure a mixture of two or more polyclonal antibodies (a polycomposition) is produced in one mixture from a polyclonal polycomposition cell line, which is produced from two or more parental polyclonal cell lines each expressing antibody molecules which are capable of binding to a distinct target, but it may also be a mixture of two or more polyclonal antibodies produced separately. A mixture of monoclonal antibodies providing the same antigen/epitope coverage as a polyclonal antibody described herein will be considered as an equivalent of a polyclonal antibody. When stating that a member of a polyclonal antibody binds to an antigen, it is herein meant to be binding with a binding constant below 100 nM, preferably below 10 nM, even more preferred below 1 nM.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following. In certain embodiments, an anti-HIV Env antibody disclosed herein binds to HIV gp120 with a Kd of at least about 0.1 M or less, at least about 0.01 M or less, at least about 1 nM or less, or at least about 0.1 nM or less. In certain embodiments, an anti-HIV Env antibody disclosed herein binds to HIV gp120 with a Kd of at least about 0.01 M or less. In certain embodiments, the HIV gp120 is BG505 gp120.

As used herein, the terms “immunospecifically binds,” “immunospecifically recognizes,” “specifically binds,” and “specifically recognizes” are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In a specific embodiment, molecules that immunospecifically bind to an antigen bind to the antigen with a Kd that is at least 2 logs, 2.5 logs, 3 logs, or 4 logs lower than the Kd when the molecules bind non-specifically to another antigen. In one example, the antibody may specifically bind to the BG505 SOSIP Env trimer. The antibody may bind to BG505 SOSIP trimer with a Kd at least 2 logs, 2.5 logs, 3 logs, or 4 logs lower than Kd of binding to other viral or non-viral polypeptides. An antibody that specifically binds to Env encompass, but are not limited to, antibodies that specifically bind to native Env, an isoform of Env, or a variant of Env (e.g., SOSIP) derived from an HIV isolate, for example, BG505. In some embodiments, the antibody specifically binds to BG505 Env. In some embodiments, the antibody or fusion polypeptide specifically binds to BG505 SOSIP.

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

An antibody is said to “competitively inhibit” binding of a reference antibody to a given epitope if it preferentially binds to that epitope or an overlapping epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

The term “broadly neutralizing antibody” or “bnAb,” as used herein, with respect to HIV (e.g., HIV-1), refers to an antibody that recognizes HIV Env of more than one isolate or strain of HIV and inhibits or prevents receptor binding of target cells as evaluated in an in vitro neutralization assay. In some embodiments, a broadly neutralizing antibody inhibits infection of a susceptible target cell by HIV. In some embodiments, a broadly neutralizing antibody specifically binds an HIV Env and inhibits infection of a susceptible target cell (e.g., TZM-bl) by an HIV pseudovirus comprising an Env polypeptide. HIV pseudovirus neutralization assays have been disclosed in the art, for example, in Walker, L. M. et al., Nature 477, 466-470 (2011), Li M., et al., J. Virol. 79:10108-10125 (2005), each of which is incorporated herein by reference in its entirety for all purposes. In some embodiments, a broadly neutralizing antibody neutralizes 2, 3, 4, 5, 6, 7, 8, 9, or more HIV strains or pseudoviruses. In some embodiments, a broadly neutralizing antibody neutralizes 2, 3, 4, 5, 6, 7, 8, 9, or more HIV strains or pseudoviruses that belong to the same or different clades. In some embodiments, a broadly neutralizing antibody is capable of neutralizing HIV strains or pseudoviruses from at least two different clades. In some embodiments, a broadly neutralizing antibody is capable of neutralizing at least one clade B strain or pseudovirus and one clade C strain or pseudovirus. In some embodiments, a broadly neutralizing antibody is capable of neutralizing more than one clade B strain or pseudovirus and more than one clade C strain or pseudovirus. In some embodiments, a broadly neutralizing antibody is capable of neutralizing an HIV strain or pseudovirus from at least one, at least two, at least three, at least four, at least five, at least six, at least seven, at least eight, at least nine, at least ten, at least eleven, at least twelve, at least thirteen, at least fourteen, or all fifteen clades represented in the 116-member indicator virus panel. In some embodiments, a broadly neutralizing antibody is capable of neutralizing an HIV strain or pseudovirus from at least one, at least two, at least three, at least four, at least five, at least six, at least seven, at least eight, at least nine, at least ten, at least eleven, at least twelve, at least thirteen, at least fourteen, or all fifteen clades selected from the group consisting of clades A, A (T/F), AC, ACD, B, B (T/F), BC, C, C (T/F), CD, CRF01_AE, CRF01_AE (T/F), CRF02_AG, D, and G. In some embodiments, a broadly neutralizing antibody is capable of neutralizing an HIV strain or pseudovirus from at least one, at least two, at least three, at least four, at least five, at least six, at least seven, at least eight, at least nine, at least ten, or all eleven clades selected from the group consisting of clades A, AC, ACD, AE, AG, B, BC, C, CD, D, G.

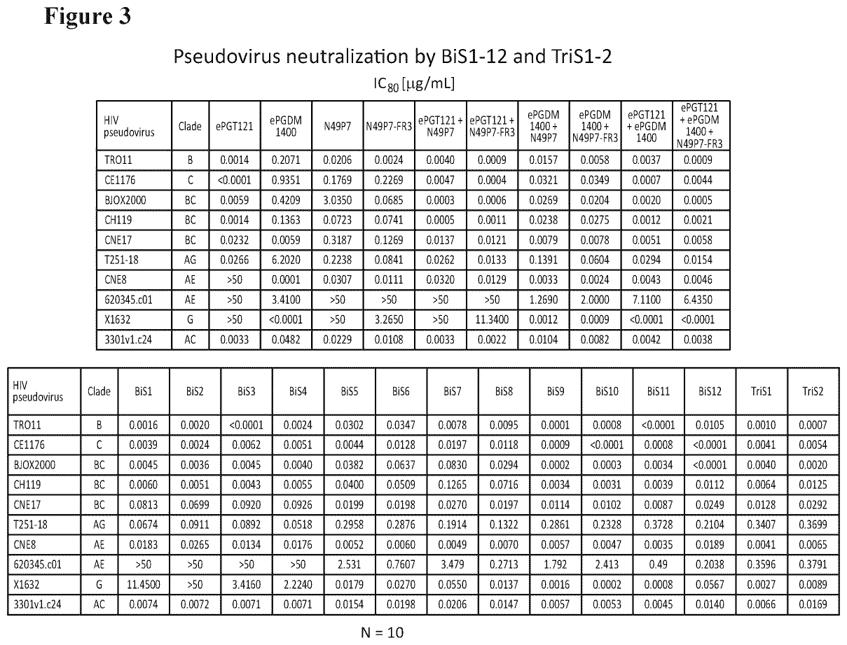

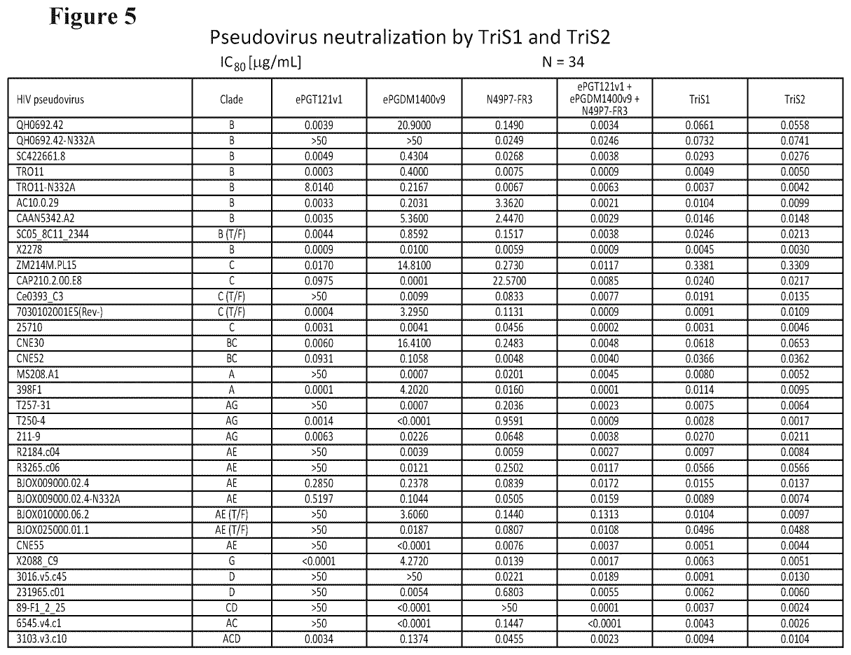

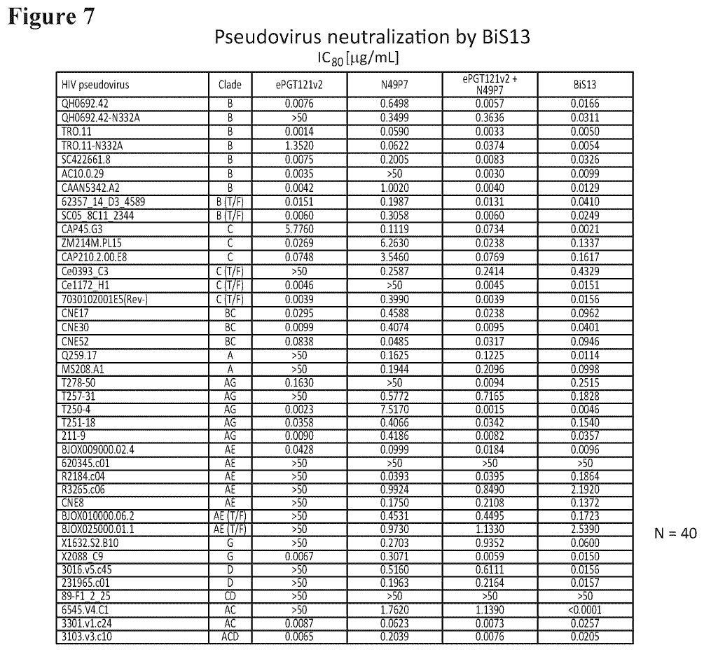

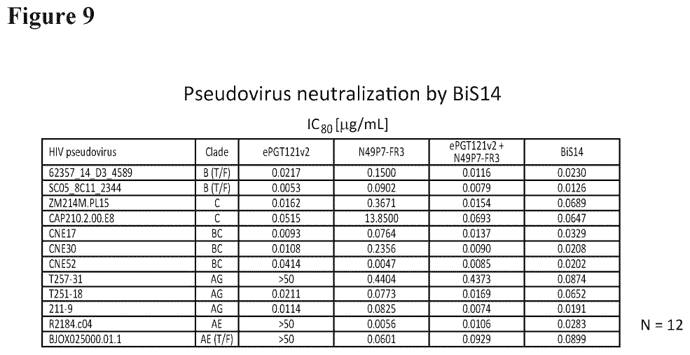

In some embodiments, the breadth of neutralization is tested on an indicator virus panel comprising cross-clade HIV isolates. In some embodiments, the virus panel comprises the 10 cross-clade isolates listed in FIG. 3. In some embodiments, the virus panel comprises the 34 cross-clade isolates listed in FIG. 5. In some embodiments, the virus panel comprises the 40 cross-clade isolates listed in FIG. 7. In some embodiments, the virus panel comprises the 12 cross-clade isolates listed in FIG. 9. In some embodiments, a broadly neutralizing antibody is capable of neutralizing at least 4, 5, 6, 7 or 8 of the cross-clade HIV isolates in the indicator virus panel. In some embodiments, a broadly neutralizing antibody is capable of neutralizing at least 5 of the cross-clade HIV isolates in the indicator virus panel. In some embodiments, a multispecific antibody described herein is a broadly neutralizing antibody.

In some embodiments, the potency of neutralization by a broadly neutralizing antibody is expressed as the median IC₅₀ neutralization activity against a virus panel. In some embodiments, the potency of neutralization by a broadly neutralizing antibody is expressed as the median IC₅₀ neutralization activity against a virus panel. In some embodiments, a broadly neutralizing antibody is capable of neutralizing at least about 4, 5, 6, 7, or 8 of the cross-clade HIV isolates in the indicator virus panel with a median IC₅₀ equal to or less than about 0.1 μg/ml, 0.07 μg/ml, 0.06 μg/ml, 0.05 μg/ml, 0.025 μg/ml, 0.01 μg/ml or 0.005 μg/ml. In some embodiments, a broadly neutralizing antibody is capable of neutralizing at least 5 of the cross-clade HIV isolates in the indicator virus panel with a median IC₅₀ equal to or less than 0.05 μg/ml.

The term “IC₅₀” refers to the half maximal inhibitory concentration of an inhibitor, e.g., a broadly neutralizing antibody. For example, IC₅₀ is the concentration of an inhibitor, e.g., a broadly neutralizing antibody, where the response, e.g., infection by pseudovirus, is reduced by half.

The phrase “substantially similar,” or “substantially the same”, as used herein, denotes a sufficiently high degree of similarity between two numeric values (generally one associated with an antibody described herein and the other associated with a reference/comparator antibody) such that one of skill in the art would consider the difference between the two values to be of little or no biological and/or statistical significance within the context of the biological characteristic measured by said values (e.g., Kd values). The difference between said two values can be less than about 50%, less than about 40%, less than about 30%, less than about 20%, or less than about 10% as a function of the value for the reference/comparator antibody.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure.

As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides described herein are based upon antibodies, in certain embodiments, the polypeptides can occur as single chains or associated chains.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin et al, Proc. Natl. Acad. Sci., 87:2264-2268 (1990), as modified in Karlin et al., Proc. Natl. Acad. Sci., 90:5873-5877 (1993), and incorporated into the NBLAST and XBLAST programs (Altschul et al., Nucleic Acids Res., 25:3389-3402 (1991)). In certain embodiments, Gapped BLAST can be used as described in Altschul et al., Nucleic Acids Res. 25:3389-3402 (1997). BLAST-2, WU-BLAST-2 (Altschul et al., Methods in Enzymology, 266:460-480 (1996)), ALIGN, ALIGN-2 (Genentech, South San Francisco, California) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48):444-453 (1970)) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS. 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, WI 53711). Bestfit uses the local homology algorithm of Smith and Waterman (Advances in Applied Mathematics 2: 482 489 (1981)) to find the best segment of homology between two sequences. When using Bestfit or any other sequence alignment program to determine whether a particular sequence is, for instance, 95% identical to a reference sequence described herein, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides described herein are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. Identity can exist over a region of the sequences that is at least about 10, about 20, about 40-60 residues in length or any integral value there between, and can be over a longer region than 60-80 residues, for example, at least about 90-100 residues, and in some embodiments, the sequences are substantially identical over the full length of the sequences being compared, such as the coding region of a nucleotide sequence for example.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In some embodiments, conservative substitutions in the sequences of the polypeptides and antibodies described herein do not abrogate the binding of the polypeptide or antibody containing the amino acid sequence, to the antigen(s). Methods of identifying nucleotide and amino acid conservative substitutions which do not eliminate antigen binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:412-417 (1997)).

As used herein, the terms “treatment” or “therapy” (as well as different forms thereof, including curative or palliative) refer to treatment of an infected person. As used herein, the term “treating” includes alleviating or reducing at least one adverse or negative effect or symptom of a condition, disease or disorder. This condition, disease or disorder can be HIV infection.

Terms such as “treating” or “treatment” or “to treat” or “alleviating” or “to alleviate” refer to therapeutic measures that cure, slow down, lessen symptoms of, and/or halt progression of a diagnosed pathologic condition or disorder, such as HIV or AIDS. Thus, those in need of treatment include those already diagnosed with or suspected of having the disorder. In certain embodiments, a subject is successfully “treated” for the disorder according to the methods described herein if the patient shows one or more of the following: a reduction in the number of or complete absence of viral load; a reduction in the viral burden; inhibition of or an absence of the virus into peripheral organs; relief of one or more symptoms associated with the disorder; reduced morbidity and mortality; improvement in quality of life; increased progression-free survival (PFS), disease-free survival (DFS), or overall survival (OS), complete response (CR), partial response (PR), stable disease (SD), a decrease in progressive disease (PD), a reduced time to progression (TTP), or any combination thereof.

As used herein, the terms “prevention” or “prophylaxis” refer to preventing a subject from becoming infected with, or reducing the risk of a subject from becoming infected with, or halting transmission of, or the reducing the risk of transmission of a virus. Prophylactic or preventative measures refer to measures that prevent and/or slow the development of a targeted pathological condition or disorder. Thus, those in need of prophylactic or preventative measures include those prone to have the disorder and those in whom the disorder is to be prevented. In some embodiments, prevention encompasses passive immunization of a subject in need thereof comprising administering an effective amount of an antibody disclosed herein.

As employed above and throughout the disclosure the term “effective amount” refers to an amount effective, at dosages, and for periods of time necessary, to achieve the desired result with respect to the treatment of the relevant disorder, condition, or side effect. An “effective amount” can be determined empirically and in a routine manner, in relation to the stated purpose. It will be appreciated that the effective amount of components of the present invention will vary from patient to patient not only with the particular vaccine, component or composition selected, the route of administration, and the ability of the components to elicit a desired result in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide an improved therapeutic response. An effective amount is also one in which any toxic or detrimental effects of the components are outweighed by the therapeutically beneficial effects.

The term “therapeutically effective amount” refers to an amount of an antibody, recombinant virus, immunoconjugate, or other drug effective to “treat” a disease or disorder in a subject or mammal. To the extent an antibody can prevent growth and/or kill existing cells, it can be cytostatic and/or cytotoxic. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The terms “subject,” “individual,” and “patient” are used interchangeably herein, and refer to an animal, for example a human, to whom treatment, including prophylactic treatment, with the antibody or pharmaceutical composition according to the present disclosure, is provided. In some embodiments, the subject, individual, or patient has been infected with HIV. In some embodiments, the subject, individual, or patient suffers from AIDS. In some embodiments, the subject, individual, or patient has been exposed to HIV. In some embodiments, the subject, individual, or patient is at risk of being exposed to HIV.

Administration “in combination with” one or more further therapeutic agents includes simultaneous (concurrent) or consecutive administration in any order.

The terms “pharmaceutically composition,” “pharmaceutical formulation,” “pharmaceutically acceptable formulation,” or “pharmaceutically acceptable composition” all of which are used interchangeably, refer to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. “Pharmaceutically acceptable” or “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The term “antiretroviral therapy” or “ART,” as used herein, refers to any of the therapies used to manage progression of a retrovirus (e.g., HIV) infection in a subject (e.g., a human), including, for example, nucleoside reverse transcriptase inhibitors (NRTIs), non-nucleoside reverse transcriptase inhibitors (NNRTIs), protease inhibitors (PIs), fusion inhibitors, entry inhibitors, maturation inhibitors, cellular inhibitors, integrase strand transfer inhibitors, and multi-class combinations. Such drugs include, but are not limited to, lamivudine and zidovudine, emtricitabine (FTC), zidovudine (ZDV), azidothymidine (AZT), lamivudine (3TC), zalcitabine, dideoxycytidine (ddC), tenofovir disoproxil fumarate (TDF), didanosine (ddl), stavudine (d4T), abacavir sulfate (ABC), etravirine, delavirdine (DLV), efavirenz (EFV), nevirapine (NVP), amprenavir (APV), tipranavir (TPV), indinavir (IDV), saquinavir, saquinavir mesylate (SQV), lopinavir (LPV), ritonavir (RTV), fosamprenavir calcium (FOS-APV), ritonavir, RTV, darunavir, atazanavir sulfate (ATV), nelfinavir mesylate (NFV), enfuvirtide, T-20, maraviroc and raltegravir. ART drugs can also include antibodies that target HIV proteins or cellular proteins associated with disease progression. Also included are immune-based therapies, such as IL-2, IL-12, and alpha-epibromide. Each of these drugs can be administered alone or in combination with any other ART drug or any HIV-specific neutralizing antibody, such as a broadly neutralizing antibody, which is incorporated by reference herein in its entirety for all purposes.

The term “reservoir activator,” as used herein, refers to an agent capable of activating a viral reservoir (e.g., an HIV reservoir). In some embodiments, a reservoir activator comprises a histone deacytelase (HDAC) inhibitor (e.g., romidepsin, vorinostat, and panobinostat), immunologic activator (e.g., cytokines and TLR agonists), or a dedicated small molecule drug.

The term “immunomodulator,” as used herein, refers to an agent, such as an antibody or peptide, which is capable of increasing, inducing, or extending an immune response (e.g., a cell-mediated immune response and/or a humoral immune response) when administered to a subject (e.g., a human, e.g., a human infected with HIV or at risk of an HIV infection or transmission). Immunomodulators include, but are not limited to immune checkpoint inhibitors, for example, a PD-1, PD-L1, LAG-3, or TIGIT antagonist. In some embodiments, an immunomodulator used in the methods described herein comprises an anti-PD-1 antibody, anti-PD-L1 antibody, anti-LAG3 antibody, or an anti-TIGIT antibody. An immunomodulator can be administered in conjunction with (e.g., prior to, concurrently with, or subsequent to, or within the context of a treatment regimen that includes the administration of a broadly neutralizing antibody described herein.

As used in this specification and the appended claims, the singular forms “a,” “an,” and “the” include plural referents unless the content clearly dictates otherwise. Thus, for example, reference to “a cell” includes a combination of two or more cells, and the like.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “about” as used herein when referring to a measurable value such as an amount, a temporal duration, and the like, is meant to encompass variations of up to ±20% from the specified value, as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope described herein are approximations, the numerical values set forth in the specific examples are reported as precisely as possible. Any numerical value, however, inherently contain certain errors necessarily resulting from the standard deviation found in their respective testing measurements.

It is understood that wherever embodiments are described herein with the language “comprising,” otherwise analogous embodiments described in terms of “consisting of” and/or “consisting essentially of” are also provided.

### II. Multispecific Antibodies

In one aspect, provided herein is a multispecific anti-HIV antibody comprising at least two antigen binding domains selected from the group consisting of (a) an antigen binding domain that binds to the V3 loop epitope region of HIV Env; (b) an antigen binding domain that binds to the V1/V2 apex epitope region of HIV Env; and (c) an antigen binding domain that binds to the CD4bs epitope region of HIV Env. In some embodiments, the multispecific antibody is a bispecific antibody. In some embodiments, the multispecific antibody is a trispecific antibody. In some embodiments, the antigen binding domain that binds to the V3 loop epitope region of HIV Env comprises a VH domain and a VL domain comprising the 3 VH CDRs and 3 VL CDRs, respectively, of PGT121 or an engineered variant of PGT121. In some embodiments, the antigen binding domain that binds to the V3 loop epitope region of HIV Env comprises a VH domain and a VL domain comprising the 3 VH CDRs and 3 VL CDRs, respectively, of ePGTT21v1 or ePGT121v2. In some embodiments, the antigen binding domain that binds to the V1/V2 apex epitope region of HIV Env comprises a VH domain and a VL domain comprising the 3 VH CDRs and 3 VL CDRs, respectively, of PGDM1400 or an engineered variant of PGDM1400. In some embodiments, the antigen binding domain that binds to the V1/V2 apex epitope region of HIV Env comprises a VH domain and a VL domain comprising the 3 VH CDRs and 3 VL CDRs, respectively, of ePGDM1400v9. In some embodiments, the antigen binding domain that binds to the CD4bs epitope region of HIV Env comprises a VH domain and a VL domain comprising the 3 VH CDRs and 3 VL CDRs, respectively, of N49P7 or an engineered variant of N49P7. In some embodiments, the antigen binding domain that binds to the CD4bs epitope region of HIV Env comprises a VH domain and a VL domain comprising the 3 VH CDRs and 3 VL CDRs, respectively, of N49P7.

TABLE 1
SEQ ID NOs of VH, VL, and CDR sequences of ePGT121v1,
ePGT121v2, ePGDM1400v9, N49P7 and N49P7 FR3

| | | | VH | VH | VH | VL | VL | VL |
|---|---|---|---|---|---|---|---|---|
| | VH | VL | CDR1 | CDR2 | CDR3 | CDR1 | CDR2 | CDR 3 |
| ePGT121v1 | 4 | 8 | 1 | 2 | 3 | 5 | 6 | 7 |
| ePGT121v2 | 22 | 26 | 19 | 20 | 21 | 23 | 24 | 25 |
| ePGDM1400v9 | 33 | 38 | 30 | 31 | 32 | 34 | 35 | 36 |
| N49P7 | 48 | 53 | 45 | 46 | 47 | 50 | 51 | 52 |
| N49P7 FR3 | 49 | 53 | 45 | 46 | 47 | 50 | 51 | 52 |

In some embodiments, the antigen binding domain that binds to the V3 loop epitope region of HIV Env comprises a VH domain and a VL domain comprising the 3 VH CDRs and 3 VL CDRs, respectively, of PGT121 or an engineered variant of PGT121. PGT121 has been disclosed, for example, in U.S. Pat. No. 9,464,131, which is incorporated by reference herein for all purposes. Engineered PGT121 variants have been disclosed, for example, in Int. Pat. Appl. Pub. No. WO/2020/023827, which is incorporated by reference herein for all purposes. In some embodiments, the VH domain comprises the VH CDR1, VH CDR2, VH CDR3 of ePGT121v1 or ePGT121v2, and the VL domain comprises the VL CDR1, VL CDR2 and VL CDR3 of ePGT121v1 or ePGT121v2. In some embodiments, the VH CDR1, VH CDR2, VH CDR3, VL CDR1, VL CDR2 and VL CDR3 comprise the amino acid sequence of SEQ ID NO: 1, 2, 3, 5, 6, and 7, respectively, or SEQ ID NO: 19, 20, 21, 23, 24 and 25, respectively. In some embodiments, the VH domain and VL domain comprises an amino acid sequence having at least 80%, at least 85%, at least 90%, at least 95%, at least 97% or 100% identity to SEQ ID NO: 4 and 8, respectively, or 22 and 26, respectively. In some embodiments, the antigen binding domain comprises an scFv domain or an Fab domain. In some embodiments, the antigen binding domain comprises an scFv comprising the amino acid sequence of SEQ ID NO: 9, 10, 11 or 12. In some embodiments, the antigen binding domain comprises an Fab domain comprising a heavy chain and light chain having the amino acid sequence of SEQ ID NO: 17 and 18, respectively, or 28 and 29, respectively.

In some embodiments, the antigen binding domain that binds to the V1/V2 apex epitope region of HIV Env comprises a VH domain and a VL domain comprising the 3 VH CDRs and 3 VL CDRs, respectively, of PGDM1400 or an engineered variant of PGDM1400. PGDM1400 has been disclosed, for example, in U.S. Pat. Appl. Pub. No. 20150361160, which is incorporated by reference herein for all purposes. Engineered PGDM1400 variants have been disclosed, for example, in Int. Pat. Appl. Pub. No. WO2021087015, which is incorporated by reference herein for all purposes. In some embodiments, the VH domain comprises the VH CDR1, VH CDR2, VH CDR3 of ePGDM1400v9, and the VL domain comprises the VL CDR1, VL CDR2 and VL CDR3 of ePGDM1400v9. In some embodiments, the VH CDR1, VH CDR2, VH CDR3, VL CDR1, VL CDR2 and VL CDR3 comprise the amino acid sequence of SEQ ID NO: 30, 31, 32, 34, 35 and 36, respectively. In some embodiments, the VH domain and VL domain comprises an amino acid sequence having at least 80%, at least 85%, at least 90%, at least 95%, at least 97% or 100% identity to SEQ ID NO: 33 and 38, respectively. In some embodiments, the antigen binding domain comprises an scFv domain or an Fab domain. In some embodiments, the antigen binding domain comprises an scFv comprising the amino acid sequence of SEQ ID NO: 39 or 40. In some embodiments, the antigen binding domain comprises an Fab domain comprising a VH and VL having the amino acid sequence of SEQ ID NO: 33 and 38, respectively.

In some embodiments, the antigen binding domain that binds to the CD4bs epitope region of HIV Env comprises a VH domain and a VL domain comprising the 3 VH CDRs and 3 VL CDRs, respectively, of N49P7 or a variant of N49P7. N49P7 has been disclosed, for example, in U.S. Pat. Appl. Pub. No. 20200172601, which is incorporated by reference herein for all purposes. N49P7 variants have been disclosed, for example, in Int. Pat. Appl. Pub. No. WO2021108761, which is incorporated by reference herein for all purposes. In some embodiments, the VH domain comprises the VH CDR1, VH CDR2, VH CDR3 of N49P7, and the VL domain comprises the VL CDR1, VL CDR2 and VL CDR3 of N49P7. In some embodiments, the VH CDR1, VH CDR2, VH CDR3, VL CDR1, VL CDR2 and VL CDR3 comprise the amino acid sequence of SEQ ID NO: 45, 46, 47, 50, 51 and 52, respectively. In some embodiments, the VH domain and VL domain comprises an amino acid sequence having at least 80%, at least 85%, at least 90%, at least 95%, at least 97% or 100% identity to SEQ ID NO: 48 and 53, respectively, or 49 and 53, respectively. In some embodiments, the antigen binding domain comprises an scFv domain or an Fab domain. In some embodiments, the antigen binding domain comprises an scFv comprising the amino acid sequence of SEQ ID NO: 54 or 55. In some embodiments, the antigen binding domain comprises an Fab domain comprising a heavy chain and light chain having the amino acid sequence of SEQ ID NO: 58 and 60, respectively, or 59 and 60, respectively.

In some embodiments, a multispecific anti-HIV antibody described herein comprises at least two antigen binding domains selected from the group consisting of (a) an antigen binding domain that binds to the V3 loop epitope region of HIV Env and comprises a VH domain and a VL domain comprising the 3 VH CDRs and 3 VL CDRs, respectively, of ePGT121v1 or ePGT121v2; (b) an antigen binding domain that binds to the V1/V2 apex epitope region of HIV Env and comprises a VH domain and a VL domain comprising the 3 VH CDRs and 3 VL CDRs, respectively, of ePGDM1400v9; and (c) an antigen binding domain that binds to the CD4bs epitope region of HIV Env and comprises a VH domain and a VL domain comprising the 3 VH CDRs and 3 VL CDRs, respectively, of N49P7. In some embodiments, the multispecific antibody is a bispecific antibody. In some embodiments, the multispecific antibody is a trispecific antibody.

In some embodiments, a multispecific anti-HIV antibody described herein comprises an scFv domain linked to an antibody, wherein the scFv domain and the antibody have different binding specificities. In some embodiments, the C-terminal end of the scFv domain is linked to the N-terminal end of the antibody light chain. In some embodiments, the linker comprises between 15 and 35 amino acid residues. In some embodiments, the linker comprises between 25 and 45 amino acid residues. In some embodiments, the multispecific antibody is a bispecific antibody. In some embodiments, the multispecific antibody is a trispecific antibody.

In some embodiments, the linker comprises between 20 and 30 amino acid residues, between 20 and 27 amino acid residues, between 23 and 30 amino acid residues, or between 23 and 27 amino acid residues. In some embodiments, the linker comprises 20, 21, 22, 23, 24, 25, 26, 27, 28, 29 or 30 amino acid residues, optionally wherein the linker comprises 23, 24, 25, 26, or 27 amino acid residues. In some embodiments, the linker comprises one or more copies of the amino acid sequence of SEQ ID NO: 141. In some embodiments, the linker comprises the amino acid sequence of SEQ ID NO: 140. In some embodiments, the multispecific antibody is a bispecific antibody. In some embodiments, the multispecific antibody is a trispecific antibody.

In some embodiments, the linker comprises between 30 and 40 amino acid residues, between 30 and 37 amino acid residues, between 33 and 40 amino acid residues, or between 33 and 37 amino acid residues. In some embodiments, the linker comprises 30, 31, 32, 33, 34, 35, 36, 37, 38, 39 or 40 amino acid residues, optionally wherein the linker comprises 33, 34, 35, 36, or 37 amino acid residues. In some embodiments, the linker comprises one or more copies of the amino acid sequence of SEQ ID NO: 141. In some embodiments, the linker comprises the amino acid sequence of SEQ ID NO: 143. In some embodiments, the multispecific antibody is a bispecific antibody. In some embodiments, the multispecific antibody is a trispecific antibody.

In some embodiments, the bispecific antibody comprises an scFv domain linked to an antibody, wherein the scFv domain and the antibody have different binding specificities. In some embodiments, the C-terminal end of the scFv domain is linked to the N-terminal end of the antibody light chain. In some embodiments, the scFv linked to the antibody light chain and the antibody heavy chain comprises an amino acid sequence having at least 80%, at least 85%, at least 90%, at least 95%, at least 97% or 100% identity to SEQ ID NO: 61 and 27, respectively, or 62 and 27, respectively.

In some embodiments, the bispecific antibody comprises an scFv domain linked to an antibody, wherein the scFv domain and the antibody have different binding specificities. In some embodiments, the C-terminal end of the scFv domain is linked to the N-terminal end of the antibody light chain. In some embodiments, the scFv linked to the antibody light chain and the antibody heavy chain comprises an amino acid sequence having at least 80%, at least 85%, at least 90%, at least 95%, at least 97% or 100% identity to SEQ ID NO: 142 and 27, respectively.

In some embodiments, the bispecific antibody comprises an scFv domain linked to a first Fc domain and an Fab domain linked to a second Fc domain, wherein the scFv and Fab domains have different binding specificities. In some embodiments, the bispecific antibody comprises a first scFv domain linked to a first Fc domain and a second scFv domain linked to a second Fc domain, wherein the first and second scFv domains have different binding specificities.

In some embodiments, the bispecific antibody comprises an scFv domain linked to a first Fc domain and an Fab domain linked to a second Fc domain, wherein the (i) scFv domain, (ii) Fab heavy chain and (iii) Fab light chain comprise an amino acid sequence having at least 80%, at least 85%, at least 90%, at least 95%, at least 97% or 100% identity to (a) SEQ ID NO: 9, 58, and 60, respectively; (b) SEQ ID NO: 10, 58, and 60, respectively; (c) SEQ ID NO: 11, 58, and 60, respectively; (d) SEQ ID NO: 12, 58, and 60, respectively; (e) SEQ ID NO: 9, 59, and 60, respectively; (f) SEQ ID NO: 10, 59, and 60, respectively; (g) SEQ ID NO: 11, 59, and 60, respectively; (h) SEQ ID NO: 12, 59, and 60, respectively; (i) SEQ ID NO: 39, 58, and 60, respectively; (j) SEQ ID NO: 40, 58, and 60, respectively; (k) SEQ ID NO: 39, 59, and 60, respectively; or (1) SEQ ID NO: 40, 59, and 60, respectively.

In some embodiments, the bispecific antibody comprises a first scFv domain linked to a first Fc domain and a second scFv domain linked to a second Fc domain, wherein the (i) first scFv domain, (ii) and second scFv domain comprise an amino acid sequence having at least 80%, at least 85%, at least 90%, at least 95%, at least 97% or 100% identity to (a) SEQ ID NO: 9 and 40, respectively; (b) SEQ ID NO: 10 and 40, respectively; (c) SEQ ID NO: 11 and 40, respectively; (d) SEQ ID NO: 12 and 40, respectively; (e) SEQ ID NO: 9 and 39, respectively; (f) SEQ ID NO: 10 and 39, respectively; (g) SEQ ID NO: 11 and 39, respectively; or (h) SEQ ID NO: 12 and 39, respectively.

In some embodiments, the first and second Fc domains are IgG Fc domains. In some embodiments, the first and/or second Fc domain has been modified to promote heterodimer formation. In some embodiments, the first Fc domain comprises the T366W substitution and the second Fc domain comprises the T366S, L368A and Y407V substitutions. In some embodiments, the first Fc domain comprises the T366S, L368A and Y407V substitutions and the second Fc domain comprises the T366W substitution. In some embodiments, the first Fc domain comprises the K409R substitution and the second Fc domain comprises the F405L substitution, or the first Fc domain comprises the F405L substitution and the second Fc domain comprises the K409R substitution.

In some embodiments, the bispecific antibody comprises an scFv domain linked to a first Fc domain and an Fab domain linked to a second Fc domain, wherein the (i) scFv domain linked to the first Fc domain, (ii) Fab heavy chain linked to the second Fc domain and (iii) Fab light chain comprise an amino acid sequence having at least 80%, at least 85%, at least 90%, at least 95%, at least 97% or 100% identity to (a) SEQ ID NO: 13, 56, and 60, respectively; (b) SEQ ID NO: 14, 56, and 60, respectively; (c) SEQ ID NO: 13, 57, and 60, respectively; (d) SEQ ID NO: 14, 57, and 60, respectively; (e) SEQ ID NO: 41, 56, and 60, respectively; (f) SEQ ID NO: 42, 56, and 60, respectively; (g) SEQ ID NO: 41, 57, and 60, respectively; or (h) SEQ ID NO: 42, 57, and 60, respectively.

In some embodiments, the bispecific antibody comprises a first scFv domain linked to a first Fc domain and a second scFv domain linked to a second Fc domain, wherein the (i) first scFv domain linked to the first Fc domain, (ii) and second scFv domain linked to the second Fc domain comprise an amino acid sequence having at least 80%, at least 85%, at least 90%, at least 95%, at least 97% or 100% identity to (a) SEQ ID NO: 13 and 44, respectively; (b) SEQ ID NO: 14 and 44, respectively; (c) SEQ ID NO: 13 and 43, respectively; or (d) SEQ ID NO: 14 and 43, respectively.

In some embodiments, a bispecific antibody described herein comprises an scFv domain linked to a first Fc domain and an Fab domain linked to a second Fc domain, wherein the (i) scFv domain linked to the first Fc domain, (ii) Fab heavy chain linked to the second Fc domain and (iii) Fab light chain comprise an amino acid sequence of SEQ ID NO: 13, 56, and 60, respectively; SEQ ID NO: 14, 56, and 60, respectively; SEQ ID NO: 13, 57, and 60, respectively; SEQ ID NO: 14, 57, and 60, respectively; SEQ ID NO: 41, 56, and 60, respectively; SEQ ID NO: 42, 56, and 60, respectively; SEQ ID NO: 41, 57, and 60, respectively; or SEQ ID NO: 42, 57, and 60, respectively.

In some embodiments, a bispecific antibody described herein comprises a first scFv domain linked to a first Fc domain and a second scFv domain linked to a second Fc domain, wherein the (i) first scFv domain linked to the first Fc domain, (ii) and second scFv domain linked to the second Fc domain comprise an amino acid sequence of SEQ ID NO: 13 and 44, respectively; SEQ ID NO: 14 and 44, respectively; SEQ ID NO: 13 and 43, respectively; or SEQ ID NO: 14 and 43, respectively.

In some embodiments, a multispecific antibody disclosed herein is a trispecific antibody. In some embodiments, a trispecific antibody disclosed herein comprises a first scFv linked to a first Fc domain, an antibody heavy chain comprising a second Fc domain, and second Fc domain linked to an antibody light chain. In some embodiments, a trispecific antibody disclosed herein comprises a first and second scFv linked to a first Fc domain, an antibody heavy chain comprising a second Fc domain, and an antibody light chain. In some embodiments, a trispecific antibody disclosed herein comprises a first scFv linked to a first Fc domain, second Fc domain linked to an antibody heavy chain comprising a second Fc domain, and an antibody light chain. In some embodiments, the first and second Fc domains are IgG Fc domains. In some embodiments, the first and/or second Fc domain has been modified to promote heterodimer formation. In some embodiments, the first Fc domain comprises the T366W substitution and the second Fc domain comprises the T366S, L368A and Y407V substitutions. In some embodiments, the first Fc domain comprises the T366S, L368A and Y407V substitutions and the second Fc domain comprises the T366W substitution. In some embodiments, the first Fc domain comprises the K409R substitution and the second Fc domain comprises the F405L substitution, or the first Fc domain comprises the F405L substitution and the second Fc domain comprises the K409R substitution.

In some embodiments, a trispecific antibody described herein comprises a first scFv linked to a first Fc domain, an antibody heavy chain comprising a second Fc domain, and second Fc domain linked to an antibody light chain, wherein the first scFv linked to a first Fc domain, the antibody heavy chain comprising a second Fc domain, and the second Fc domain linked to an antibody light chain comprise an amino acid sequence having at least 80%, at least 85%, at least 90%, at least 95%, at least 97% or 100% identity to SEQ ID NO: 41, 16 and 142, respectively, or 42, 16 and 142, respectively.

In some embodiments, a trispecific antibody described herein comprises a first and second scFv linked to a first Fc domain, an antibody heavy chain comprising a second Fc domain, and an antibody light chain, wherein the first and second scFv linked to a first Fc domain, the antibody heavy chain comprising a second Fc domain, and the antibody light chain comprise an amino acid sequence having at least 80%, at least 85%, at least 90%, at least 95%, at least 97% or 100% identity to SEQ ID NO: 65, 57 and 60, respectively, or 66, 57 and 60, respectively.

In some embodiments, a trispecific antibody described herein comprises a first scFv linked to a first Fc domain, second Fc domain linked to an antibody heavy chain comprising a second Fc domain, and an antibody light chain, wherein the first scFv linked to a first Fc domain, the second Fc domain linked to an antibody heavy chain comprising a second Fc domain, and the antibody light chain comprise an amino acid sequence having at least 80%, at least 85%, at least 90%, at least 95%, at least 97% or 100% identity to SEQ ID NO: 13, 67 and 60, respectively, or 13, 68 and 60, respectively.

In some embodiments, a bispecific antibody described herein is BiS1. BiS 2, BiS 3, BiS 4, BiS 5, BiS 6, BiS 7, BiS 8, BiS 9, BiS 10, BiS 11, BiS 12, BiS 13 or BiS 14. In some embodiments, a trispecific antibody described herein is TriS 1, TriS 2, TriS 3, TriS 4, TriS 5 or TriS 6.

TABLE 2
Polypeptide chains of exemplary multispecific antibodies

| Multispecific | | | |
|---|---|---|---|
| antibody | Chain 1 | Chain 2 | Chain 3 |
| BiS1 | SEQ ID NO: 13 | SEQ ID NO: 56 | SEQ ID NO: 60 |
| BiS 2 | SEQ ID NO: 14 | SEQ ID NO: 56 | SEQ ID NO: 60 |
| BiS 3 | SEQ ID NO: 13 | SEQ ID NO: 57 | SEQ ID NO: 60 |
| BiS 4 | SEQ ID NO: 14 | SEQ ID NO: 57 | SEQ ID NO: 60 |
| BiS 5 | SEQ ID NO: 41 | SEQ ID NO: 56 | SEQ ID NO: 60 |
| BiS 6 | SEQ ID NO: 42 | SEQ ID NO: 56 | SEQ ID NO: 60 |
| BiS 7 | SEQ ID NO: 41 | SEQ ID NO: 57 | SEQ ID NO: 60 |
| BiS 8 | SEQ ID NO: 42 | SEQ ID NO: 57 | SEQ ID NO: 60 |
| BiS 9 | SEQ ID NO: 13 | SEQ ID NO: 44 | N/A |
| BiS 10 | SEQ ID NO: 14 | SEQ ID NO: 44 | N/A |
| BiS 11 | SEQ ID NO: 13 | SEQ ID NO: 43 | N/A |
| BiS 12 | SEQ ID NO: 14 | SEQ ID NO: 43 | N/A |
| BiS 13 | SEQ ID NO: 27 | SEQ ID NO: 61 | N/A |
| BiS 14 | SEQ ID NO: 27 | SEQ ID NO: 62 | N/A |
| TriS 1 | SEQ ID NO: 41 | SEQ ID NO: 16 | SEQ ID NO: 142 |
| TriS 2 | SEQ ID NO: 42 | SEQ ID NO: 16 | SEQ ID NO: 142 |
| TriS 3 | SEQ ID NO: 65 | SEQ ID NO: 57 | SEQ ID NO: 60 |
| TriS 4 | SEQ ID NO: 66 | SEQ ID NO: 57 | SEQ ID NO: 60 |
| TriS 5 | SEQ ID NO: 13 | SEQ ID NO: 67 | SEQ ID NO: 60 |
| TriS 6 | SEQ ID NO: 13 | SEQ ID NO: 68 | SEQ ID NO: 60 |

Also provided herein are polypeptides that comprise an amino acid sequence having at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence, or is identical to the sequences listed in Table 2. In some embodiments, the polypeptides comprise the 3 VH CDRs and 3 VL CDRs of ePGT121 v1, ePGT121 v2, ePGDM1400v9 or N49P7.

In some embodiments, a multispecific (e.g., bispecific) antibody described herein is a recombinant antibody, a chimeric antibody, a bispecific antibody, or a trispecific antibody. In some embodiments, the antibody fragment comprises a F(ab′)2 fragment.

In some embodiments, a multispecific antibody described herein is capable of neutralizing the BG505 HIV isolate.

In some embodiments, a multispecific antibody described herein is capable of neutralizing at least two cross-clade isolates of HIV. In one embodiment, the antibody is capable of neutralizing at least one clade B isolate and at least one clade C isolate.

In some embodiments, a multispecific antibody described herein is a broadly neutralizing antibody.

In some embodiments, a multispecific antibody described herein neutralizes 2, 3, 4, 5, 6, 7, 8, 9, or more HIV strains or pseudoviruses that belong to the same or different clades. In some embodiments, a multispecific antibody described herein is capable of neutralizing HIV strains or pseudoviruses from at least two different clades. In some embodiments, a multispecific antibody described herein is capable of neutralizing at least one clade B strain or pseudovirus and one clade C strain or pseudovirus. In some embodiments, a multispecific antibody described herein is capable of neutralizing more than one clade B strain or pseudovirus and more than one clade C strain or pseudovirus. In some embodiments, a multispecific antibody described herein is capable of neutralizing an HIV strain or pseudovirus from at least one, at least two, at least three, at least four, at least five, at least six, at least seven, at least eight, at least nine, at least ten, at least eleven, at least twelve, at least thirteen, at least fourteen, or fifteen clades selected from the group consisting of clades A, A (T/F), AC, ACD, B, B (T/F), BC, C, C(T/F), BC, CD, AE, AE (T/F), AG, D, and G.

In some embodiments, a multispecific antibody described herein is capable of neutralizing at least 90% of the cross-clade HIV isolates in the indicator virus panel of FIG. 3. In some embodiments, a multispecific antibody described herein is capable of neutralizing at least 90% of the cross-clade HIV isolates in the indicator virus panel of FIG. 5. In some embodiments, a multispecific antibody described herein is capable of neutralizing at least 90% of the cross-clade HIV isolates in the indicator virus panel of FIG. 7. In some embodiments, a multispecific antibody described herein is capable of neutralizing at least 90% of the cross-clade HIV isolates in the indicator virus panel of FIG. 9.

In some embodiments, a multispecific antibody described herein is capable of neutralizing the cross-clade HIV isolates in the indicator virus panel of FIG. 3. In some embodiments, a multispecific antibody described herein is capable of neutralizing the cross-clade HIV isolates in the indicator virus panel of FIG. 5. In some embodiments, a multispecific antibody described herein is capable of neutralizing the cross-clade HIV isolates in the indicator virus panel of FIG. 7. In some embodiments, a multispecific antibody described herein is capable of neutralizing the cross-clade HIV isolates in the indicator virus panel of FIG. 9.

Techniques for making multispecific antibodies are known by those skilled in the art, see for example, Millstein et al., 1983, Nature, 305:537-539; Brennan et al., 1985, Science, 229:81; Suresh et al., 1986, Methods in Enzymol., 121:120; Traunecker et al., 1991, EMBO J., 10:3655-3659; Shalaby et al., 1992, J. Exp. Med., 175:217-225; Kostelny et al., 1992, J. Immunol., 148:1547-1553; Gruber et al., 1994, J. Immunol., 152:5368; U.S. Pat. No. 5,731,168; International Publication No. WO 2009/089004; and U.S. Patent Publication No. 2011/0123532. In some embodiments, the multispecific antibodies comprise heavy chain constant regions with modifications in the amino acids which are part of the interface between the two heavy chains.

In some embodiments, multispecific antibodies described herein comprise asymmetric heavy chain constant regions that promote heterodimer formation, including in “knob-in-hole” structures. See, Kontermann, MAbs., 4(2):182-97 (2012). Knobs-into-holes (KIHs) technology involves engineering CH3 domains to create either a “knob” or a “hole” in each heavy chain to promote heterodimerization. KIH technology is described, for instance, in Ridgway et al., Protein Engineering 9(7):617-721 (1996); U.S. Pat. Nos. 5,731,168; 5,807,706; 5,821,333, each of which is herein incorporated by reference in its entirety. The knobs-into-holes approach introduces amino acids with bulky side chains into the CH3 domain of one heavy chain that fit into appropriately designed cavities in the CH3 domain of the other heavy chain. The combination of approaches prevents mismatch of both heavy chain to heavy chain and heavy chain to light chain interactions, resulting in primarily a single product. At times the “knobs” and “holes” terminology is replaced with the terms “protuberances” and “cavities”. In some embodiments, the multispecific antibodies may comprise variant hinge regions incapable of forming disulfide linkages between the heavy chains (see, e.g., WO 2006/028936).

In some embodiments, a multispecific antibody described herein comprises a first and second heavy chain or Fc region. In some embodiments, a multispecific antibody described herein comprises a human heavy and/or light chain constant region. In some embodiments, the heavy chain constant region is human immunoglobulin IgG1, IgG2, IgG3, IgG4, IgA1, or IgA2 constant region. In some embodiments, the heavy chain constant region is human immunoglobulin IgG1 constant region. In some embodiments, the heavy chain constant region is human immunoglobulin IgG4 constant region. In some embodiments, the heavy chain constant region is human immunoglobulin IgA1 constant region. In some embodiments, the heavy chain constant region is human immunoglobulin IgA2 constant region. In some embodiments, the heavy chain constant region comprises a native amino acid sequence. In some embodiments, the heavy chain constant region comprises a variant amino acid sequence. In some embodiments, the first and second heavy chain comprises IgG Fc domains. In some embodiments, the first and second heavy chain comprises IgG1 Fc domains. In some embodiments, the first and second heavy chain comprises IgG4 Fc domains. In some embodiments, the first and second heavy chain comprises IgA Fc domains. In some embodiments, the first and second heavy chain comprises IgA1 Fc domains. In some embodiments, the first and second heavy chain comprises IgA2 Fc domains.

In some embodiments, a multispecific antibody described herein comprises a first and second heavy chain comprising a first and second VH domain described herein and a common light chain comprising a common light chain described herein, wherein the first and second heavy chains comprise first and second Fc domains that comprising modifications to promote heterodimer formation. In some embodiments, the first Fc domain comprises the T366W substitution and the second Fc domain comprises the T366S, L368A and Y407V substitutions, wherein the first and second Fc domains are IgG Fc domains. In some embodiments, the first Fc domain comprises the T366S, L368A and Y407V substitutions and the second Fc domain comprises the T366W substitution, wherein the first and second Fc domains are IgG Fe domains. In some embodiments, the first and second Fc domains are IgG Fc domains, wherein (1) the first Fc domain comprises the K409R substitution and the second Fc domain comprises the F405L substitution; or (2) the first Fc domain comprises the F405L substitution and the second Fc domain comprises the K409R substitution. In some embodiments, the first and second Fc domains are modified, wherein (1) the first Fc domain comprises a substitution corresponding to the K409R substitution of the IgG Fc domain and the second Fc domain comprises a substitution corresponding to the F405L substitution of the IgG Fc domain; or (2) the first Fc domain comprises a substitution corresponding to the F405L substitution of the IgG Fc domain and the second Fc domain comprises a substitution corresponding to the K409R substitution of the IgG Fc domain.

In some embodiments, a multispecific (e.g., multispecific) antibody described herein can be made using recombinant DNA methods, for example, as described in U.S. Pat. Nos. 5,731,168; 5,807,706; 5,821,333, each of which is herein incorporated by reference in its entirety. The polynucleotides encoding a multispecific (e.g., bispecific) antibody can be amplified from a suitable source or chemically synthetized. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells.

The polynucleotide(s) encoding a multispecific (e.g., bispecific) antibody described herein can be modified in a number of different manners using recombinant DNA technology to generate alternative antibodies. In some embodiments, the constant domains of the light and heavy chains can be substituted for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

Methods for engineering antibodies can also be used and are well known in the art. An engineered antibody can have one or more amino acid residues substituted, deleted or inserted. These sequence modifications can be used to reduce immunogenicity or reduce, enhance or modify binding, affinity, on-rate, off-rate, avidity, specificity, half-life, or any other suitable characteristic, as known in the art. Antibodies can also be engineered to eliminate development liabilities by altering or eliminating sequence elements targeted for post-translational modification including glycosylation sites, oxidation sites, or deamination sites. In general, the CDR residues are directly and most substantially involved in influencing antibody binding. Accordingly, part or all of the CDR sequences are maintained while the variable framework and constant regions can be engineered by introducing substitutions, insertions, or deletions.

Antibodies disclosed herein can also optionally be engineered with retention of high affinity for the antigen and other favorable biological properties. To achieve this goal, engineered antibodies can be prepared by a process of analysis of the parental sequences and various conceptual engineered products using three-dimensional models of the parental and engineered sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved.

In certain embodiments an antibody fragment, e.g., F(ab′)2 is provided. Various techniques are known for the production of antibody fragments. Traditionally, these fragments are derived via proteolytic digestion of intact antibodies (for example Morimoto et al., 1993, Journal of Biochemical and Biophysical Methods 24:107-117; Brennan et al., 1985, Science, 229:81). In certain embodiments, antibody fragments are produced recombinantly. F(ab′)2 antibody fragments can be expressed in host cells to allowing the production of large amounts of these fragments. Such antibody fragments can also be isolated from antibody phage libraries. The antibody fragment can also be linear antibodies as described in U.S. Pat. No. 5,641,870, for example, and can be monospecific or bispecific. Other techniques for the production of antibody fragments will be apparent to the skilled practitioner.

In certain embodiments, the variable domains in both the heavy and light chains are altered by at least partial replacement of one or more CDRs and, if necessary, by partial framework region replacement and sequence changing. Although the CDRs can be derived from an antibody of the same class or even subclass as the antibody from which the framework regions are derived, it is envisaged that the CDRs will be derived from an antibody of different class and in certain embodiments from an antibody from a different species. It may not be necessary to replace all of the CDRs with the complete CDRs from the donor variable region to transfer the antigen-binding capacity of one variable domain to another. Rather, it may only be necessary to transfer those residues that are necessary to maintain the activity of the antigen-binding site. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

Those skilled in the art will appreciate that the antibodies described herein will comprise antibodies (e.g., full-length antibodies or antigen-binding fragments thereof) in which at least a fraction of one or more of the constant region domains has been deleted or otherwise altered so as to provide desired biochemical characteristics such as increased serum half-life when compared with an antibody of approximately the same antigen-binding activity comprising a native or unaltered constant region. In some embodiments, the constant region of the antibodies will comprise a human constant region. Modifications to the constant region compatible with this invention comprise additions, deletions or substitutions of one or more amino acids in one or more domains. That is, the antibodies described herein can comprise alterations or modifications to one or more of the three heavy chain constant domains (CH1, CH2 or CH3) and/or to the light chain constant domain (CL). In some embodiments, modified constant regions wherein one or more domains are partially or entirely deleted are contemplated. In some embodiments, the antibodies will comprise domain deleted constructs or variants wherein the entire CH2 domain has been removed (ΔCH2 constructs). In some embodiments, the omitted constant region domain will be replaced by a short amino acid spacer (e.g., 10 residues) that provides some of the molecular flexibility typically imparted by the absent constant region.

It will be noted that in certain embodiments, the antibodies can be engineered to fuse the CH3 domain directly to the hinge region of the respective antibodies. In other constructs it may be desirable to provide a peptide spacer between the hinge region and the modified CH2 and/or CH3 domains. For example, compatible constructs could be expressed wherein the CH2 domain has been deleted and the remaining CH3 domain (modified or unmodified) is joined to the hinge region with a 5-20 amino acid spacer. Such a spacer can be added, for instance, to ensure that the regulatory elements of the constant domain remain free and accessible or that the hinge region remains flexible. However, it should be noted that amino acid spacers can, in some cases, prove to be immunogenic and elicit an unwanted immune response against the construct. Accordingly, in certain embodiments, any spacer added to the construct will be relatively non-immunogenic, or even omitted altogether, so as to maintain the desired biochemical qualities of the antibodies.

Besides the deletion of whole constant region domains, it will be appreciated that the antibodies described herein can be provided by the partial deletion or substitution of a few or even a single amino acid. For example, it may be desirable to simply delete that part of one or more constant region domains that control the effector function (e.g., complement C1Q binding) to be modulated. Such partial deletions of the constant regions can improve selected characteristics of the antibody (serum half-life) while leaving other desirable functions associated with the subject constant region domain intact. Moreover, as alluded to above, the constant regions of the disclosed antibodies can be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the antibody. Certain embodiments can comprise the addition of one or more amino acids to the constant region to enhance desirable characteristics such as decreasing or increasing effector function or provide for more cytotoxin or carbohydrate attachment. In such embodiments it can be desirable to insert or replicate specific sequences derived from selected constant region domains. In further embodiments, an antibody disclosed herein comprises a variant IgG Fc region (e.g., variant IgG1 Fc region) comprising the M428L and N434S substitutions to improve the recycling of the antibody via the antibody salvage pathway. See, e.g., Grevys, et al., The Journal of Immunology, 194(11):5497-508 (2015). In some embodiments, an antibody disclosed herein comprises the variant IgG Fc region of SEQ ID NO: 110.

In some embodiments, an antibody used in a method disclosed herein comprises a variant IgG Fc region that has been modified to increase half-life. The half-life of an IgG is mediated by its pH-dependent binding to the neonatal receptor FcRn. In some embodiments, the Fc has been modified to enhance binding to FcRn (see, e.g., Petkova et al., Int. Immunol. 18: 1759-1769 (2006); Dall'Acqua et al., J. Immunol.169: 5171-5180 (2002); Oganesyan et al., Mol. Immunol. 46: 1750-1755 (2009); Dall'Acqua et al., J. Biol. Chem. 281: 23514-23524 (2006), Hinton et al., J. Immunol. 176: 346-356 (2006); Datta-Mannan et al., Drug Metab. Dispos. 35: 86-94 (2007); Datta-Mannan et al., J. Biol. Chem. 282: 1709-1717 (2007); WO 06/130834; Strohl, Curr. Op. Biotechnol. 20: 685-691 (2009); and Yeung et al., J. Immunol. 182: 7663-7671 (2009), the contents of each of which is herein incorporated by reference in its entirety). In some embodiments, the modification to the Fc region comprises one or more of the following modifications that increase half-life: IgG1-M252Y, S254T, T256E; IgG1-T250Q, M428L; IgG1-H433K, N434Y; IgG1-N434A; and IgG1-T307A, E380A, N434A; wherein the numbering of the residues is that of the EU index of Kabat et al. (Kabat et al., Sequences of Proteins of Immunological Interest, 1991 Fifth edition, herein incorporated by reference). In some embodiments, the modification to the Fc region comprises the M428L/N434S substitution.

The present invention further embraces variants and equivalents which are substantially homologous to the antibodies, or antibody fragments thereof, set forth herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

The antibodies described herein can be further modified to contain additional chemical moieties not normally part of the protein. Those derivatized moieties can improve the solubility, the biological half-life or absorption of the protein. The moieties can also reduce or eliminate any desirable side effects of the proteins and the like. An overview for those moieties can be found in REMINGTON'S PHARMACEUTICAL SCIENCES, 21th ed., Mack Publishing Co., Easton, PA (2005).

### II. Polynucleotides

In certain aspects, provided herein are polynucleotides comprising a nucleotide sequence or nucleotide sequences encoding a multispecific antibody described herein and vectors, e.g., vectors comprising such polynucleotides. In one embodiment, the vectors can be used for recombinant expression of a multispecific antibody described herein in host cells (e.g., E. coli and mammalian cells). In one embodiment, the vectors can be used for administration of a multispecific antibody described herein to a patient in need thereof. In some embodiment, the multispecific antibody is BiS13 or BiS14. In some embodiment, the multispecific antibody is BiS13.

In some embodiments, a polynucleotide encoding a bispecific antibody described herein comprises 3 polynucleotides comprising the nucleotide sequence of SEQ ID NO: 82-84. In some embodiments, a polynucleotide encoding a bispecific antibody described herein comprises 3 polynucleotides comprising the nucleotide sequence of SEQ ID NO: 85-87. In some embodiments, a polynucleotide encoding a bispecific antibody described herein comprises 3 polynucleotides comprising the nucleotide sequence of SEQ ID NO: 88-90. In some embodiments, a polynucleotide encoding a bispecific antibody described herein comprises 3 polynucleotides comprising the nucleotide sequence of SEQ ID NO: 91-93. In some embodiments, a polynucleotide encoding a bispecific antibody described herein comprises 3 polynucleotides comprising the nucleotide sequence of SEQ ID NO: 94-96. In some embodiments, a polynucleotide encoding a bispecific antibody described herein comprises 3 polynucleotides comprising the nucleotide sequence of SEQ ID NO: 97-99. In some embodiments, a polynucleotide encoding a bispecific antibody described herein comprises 3 polynucleotides comprising the nucleotide sequence of SEQ ID NO: 100-102. In some embodiments, a polynucleotide encoding a bispecific antibody described herein comprises 3 polynucleotides comprising the nucleotide sequence of SEQ ID NO: 103-105. In some embodiments, a polynucleotide encoding a bispecific antibody described herein comprises 2 polynucleotides comprising the nucleotide sequence of SEQ ID NO: 106 and 107. In some embodiments, a polynucleotide encoding a bispecific antibody described herein comprises 2 polynucleotides comprising the nucleotide sequence of SEQ ID NO: 108 and 109. In some embodiments, a polynucleotide encoding a bispecific antibody described herein comprises 2 polynucleotides comprising the nucleotide sequence of SEQ ID NO: 110 and 111. In some embodiments, a polynucleotide encoding a bispecific antibody described herein comprises 2 polynucleotides comprising the nucleotide sequence of SEQ ID NO: 112 and 113. In some embodiments, a polynucleotide encoding a bispecific antibody described herein comprises 2 polynucleotides comprising the nucleotide sequence of SEQ ID NO: 114 and 115. In some embodiments, a polynucleotide encoding a bispecific antibody described herein comprises 2 polynucleotides comprising the nucleotide sequence of SEQ ID NO: 116 and 117. In some embodiments, the polynucleotides encoding a bispecific antibody described herein further comprise a nucleotide sequence encoding a signal peptide. In some embodiments, the signal peptide comprises the amino acid sequence of SEQ ID NO: 69 or 70. In some embodiments, the nucleotide sequence encoding a signal peptide comprises SEQ ID NO: 136 or 137.

In some embodiments, a polynucleotide encoding a trispecific antibody described herein comprises 3 polynucleotides comprising the nucleotide sequence of SEQ ID NO: 118-120. In some embodiments, a polynucleotide encoding a trispecific antibody described herein comprises 3 polynucleotides comprising the nucleotide sequence of SEQ ID NO: 121-123. In some embodiments, a polynucleotide encoding a trispecific antibody described herein comprises 3 polynucleotides comprising the nucleotide sequence of SEQ ID NO: 124-126. In some embodiments, a polynucleotide encoding a trispecific antibody described herein comprises 3 polynucleotides comprising the nucleotide sequence of SEQ ID NO: 127-129. In some embodiments, a polynucleotide encoding a trispecific antibody described herein comprises 3 polynucleotides comprising the nucleotide sequence of SEQ ID NO: 130-132. In some embodiments, a polynucleotide encoding a trispecific antibody described herein comprises 3 polynucleotides comprising the nucleotide sequence of SEQ ID NO: 133-135. In some embodiments, the polynucleotides encoding a bispecific antibody described herein further comprise a nucleotide sequence encoding a signal peptide. In some embodiments, the signal peptide comprises the amino acid sequence of SEQ ID NO: 69 or 70. In some embodiments, the nucleotide sequence encoding a signal peptide comprises SEQ ID NO: 136 or 137.

In one embodiment, an isolated polynucleotide described herein encodes a multispecific antibody described herein and comprises an mRNA. In one embodiment, the mRNA comprises at least one modified nucleotide. In one embodiment, a modified mRNA encoding an antibody disclosed herein is for administering to a subject to treat or prevent HIV infection.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1% (in particular less than about 10%) of other material, e.g., cellular material, culture medium, other nucleic acid molecules, chemical precursors and/or other chemicals. In a specific embodiment, a nucleic acid molecule(s) encoding an antibody or fusion polypeptide described herein is isolated or purified.

In particular aspects, provided herein are polynucleotides comprising nucleotide sequences encoding antibodies described herein, as well as antibodies that compete with such antibodies for binding to HIV, or which binds to the same epitope as that of such antibodies.

In yet another specific embodiment, a polynucleotide provided herein comprises a nucleotide sequence encoding an antibody described herein, which immunospecifically binds Env, wherein the antibody comprises a VL domain and a VH domain comprising any amino acid sequences described herein, and wherein the constant regions comprise the amino acid sequences of the constant regions of a human IgA1, human IgA2′ human IgG1 (e.g., allotype 1, 17, or 3), human IgG2, or human IgG4.

In yet another specific embodiment, a polynucleotide provided herein comprises a nucleotide sequences encoding an anti-Env antibody or a polypeptide thereof that are optimized, e.g., by codon/RNA optimization, replacement with heterologous signal sequences, and elimination of mRNA instability elements. Methods to generate optimized nucleic acids encoding an anti-Env antibody or a polypeptide thereof for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid.

In certain embodiments, an optimized polynucleotide sequence encoding an anti-Env antibody described herein or a polypeptide thereof can hybridize to an antisense (e.g., complementary) polynucleotide of an unoptimized polynucleotide sequence encoding an anti-Env antibody described herein or a polypeptide. In specific embodiments, an optimized nucleotide sequence encoding an anti-Env antibody described herein or a polypeptide thereof hybridizes under high stringency conditions to antisense polynucleotide of an unoptimized polynucleotide sequence encoding an anti-Env antibody described herein or a polypeptide thereof. In a specific embodiment, an optimized nucleotide sequence encoding an anti-Env antibody described herein or a polypeptide thereof hybridizes under high stringency, intermediate or lower stringency hybridization conditions to an antisense polynucleotide of an unoptimized nucleotide sequence encoding an anti-Env antibody described herein or a polypeptide thereof. Information regarding hybridization conditions has been described, see, e.g., U.S. Patent Application Publication No. US 2005/0048549 (e.g., paragraphs 72-73), which is incorporated herein by reference.

The polynucleotides can be obtained, and the nucleotide sequence of the polynucleotides determined, by any method known in the art. Nucleotide sequences encoding antibodies described herein, and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier G et al., (1994), BioTechniques 17: 242-246), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody or polypeptide thereof described herein can be generated from nucleic acid from a suitable source (e.g., PBMCs) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning, for example, to generate chimeric and humanized antibodies.

If a clone containing a nucleic acid encoding a particular antibody or polypeptide thereof is not available, but the sequence of the antibody molecule or polypeptide thereof is known, a nucleic acid encoding the immunoglobulin or polypeptide can be chemically synthesized or obtained from a suitable source (e.g., an antibody cDNA library or a cDNA library generated from, or nucleic acid, preferably poly A+ RNA, isolated from, any tissue or cells expressing the antibody, such as hybridoma cells selected to express an antibody described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding anti-HIV Env antibodies described herein can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of anti-Env antibodies in the recombinant host cells.

The DNA also can be modified, for example, by substituting the coding sequence for human heavy and light chain constant domains with a coding sequence for a non-immunoglobulin polypeptide, or by covalently joining to the immunoglobulin coding sequence all or part of the coding sequence for a non-immunoglobulin polypeptide.

### IV. Vectors, Cells, and Methods of Producing a Multispecific Antibody

In certain aspects, provided herein are cells (e.g., host cells) expressing (e.g., recombinantly) multispecific antibodies described herein which specifically bind to HIV Env and related polynucleotides and expression vectors. Provided herein are vectors (e.g., expression vectors) comprising polynucleotides comprising nucleotide sequences encoding anti-HIV Env antibodies or a fragment thereof described herein. In one embodiment, the vectors can be used for recombinant expression of an antibody described herein in host cells (e.g., mammalian cells). In one embodiment, the vectors can be used for administration of an antibody described herein to a patient in need thereof. Also provided herein are host cells comprising such vectors for recombinantly expressing anti-HIV Env antibodies described herein. In a particular aspect, provided herein are methods for producing an antibody described herein, comprising expressing such antibody in a host cell. In some embodiment, the multispecific antibody is BiS13 or BiS14. In some embodiment, the multispecific antibody is BiS13.

In certain aspects, provided herein is an isolated vector comprising a polynucleotide described herein. In one embodiment, the vector is a viral vector.

In certain aspects, provided herein is a recombinant virus comprising a polynucleotide described herein. In one embodiment, the recombinant virus encodes an antibody described herein. In one embodiment, the recombinant virus encodes a multispecific (e.g. bispecific or trispecific) antibody described herein. In one embodiment, the recombinant virus is a replication defective virus. Suitable replication defective viral vectors are known to those skilled in the art, for example, as disclosed in U.S. Pat. Nos. 7,198,784, 9,408,905, 9,862,931, 8,067,156, U.S. Pat. Appl. Pub. Nos. 20150291935, 20120220492, 20180291351, and 20170175137, each of which is incorporated herein by reference in its entirety. In one embodiment, the recombinant virus is a retrovirus or retroviral vector, for example, a lentivirus or lentiviral vector. In one embodiment, the recombinant virus is an adenovirus or adenoviral vector, HSV or HSV vector, or influenza virus or viral vector. In one embodiment, the recombinant virus is an adeno-associated virus (AAV). In one embodiment, the recombinant virus is for administration to a subject to prevent or treat HIV infection. In one embodiment, the recombinant virus is an adeno-associated virus (AAV) for administration to a subject to prevent or treat HIV infection. Recombinant AAV particles encoding an antibody that binds to HIV Env and methods for producing thereof are known to one skilled in the art, for example, as disclosed in U.S. Pat. No. 8,865,881 and US20190031740, each of which is incorporated by reference herein in its entirety for all purposes. See also, Lin and Balazs, Retrovirology 15:66 (2018) and van den berg et al., Molecular Therapy: methods & Clinical Development 14:100-112 (2019), each of which is incorporated by reference herein in its entirety for all purposes.

In certain aspects, provided herein is a host cell comprising a polynucleotide described herein, or a vector described herein. In one embodiment, the vector encodes a multispecific antibody described herein. In one embodiment, a vector described herein comprises a first vector encoding a first polypeptide of a multispecifc (e.g. bispecific or trispecific) antibody described herein and a second vector encoding a second polypeptide of the antibody described herein. In one embodiment, a vector described herein comprises a first, second and third vector encoding a first, second and third polypeptide of a multispecifc antibody described herein. In one embodiment, a vector described herein comprises a first nucleotide sequence encoding a first polypeptide of a multispecifc antibody described herein and a second nucleotide sequence encoding a second polypeptide of the antibody described herein. In one embodiment, a vector described herein comprises a first, second and third nucleotide sequence encoding a first, second and third polypeptide of a multispecific antibody described herein. In some embodiments, a polypeptide of a multispecifc antibody described herein comprises a signal peptide. In some embodiments, the nucleotide sequence encoding a polypeptide of an antibody described herein comprises a nucleotide sequence encoding a signal peptide. In some embodiments, the signal peptide comprises the amino acid sequence of SEQ ID NO: 69 or 70. In some embodiments, the nucleotide sequence encoding the signal peptide comprises SEQ ID NO: 136 or 137.

In some embodiments, a vector encoding a bispecific antibody described herein comprises the nucleotide sequence of SEQ ID NO: 82-84. In some embodiments, a vector encoding a bispecific antibody described herein comprises the nucleotide sequence of SEQ ID NO: 85-87. In some embodiments, a vector encoding a bispecific antibody described herein comprises the nucleotide sequence of SEQ ID NO: 88-90. In some embodiments, a vector encoding a bispecific antibody described herein comprises the nucleotide sequence of SEQ ID NO: 91-93. In some embodiments, a vector encoding a bispecific antibody described herein comprises the nucleotide sequence of SEQ ID NO: 94-96. In some embodiments, a vector encoding a bispecific antibody described herein comprises the nucleotide sequence of SEQ ID NO: 97-99. In some embodiments, a vector encoding a bispecific antibody described herein comprises the nucleotide sequence of SEQ ID NO: 100-102. In some embodiments, a vector encoding a bispecific antibody described herein comprises the nucleotide sequence of SEQ ID NO: 103-105. In some embodiments, a vector encoding a bispecific antibody described herein comprises the nucleotide sequence of SEQ ID NO: 106 and 107. In some embodiments, a vector encoding a bispecific antibody described herein comprises the nucleotide sequence of SEQ ID NO: 108 and 109. In some embodiments, a vector encoding a bispecific antibody described herein comprises the nucleotide sequence of SEQ ID NO: 110 and 111. In some embodiments, a vector encoding a bispecific antibody described herein comprises the nucleotide sequence of SEQ ID NO: 112 and 113. In some embodiments, a vector encoding a bispecific antibody described herein comprises the nucleotide sequence of SEQ ID NO: 114 and 115. In some embodiments, a vector encoding a bispecific antibody described herein comprises the nucleotide sequence of SEQ ID NO: 116 and 117. In some embodiments, the polynucleotides encoding a bispecific antibody described herein further encode a signal peptide. In some embodiments, the signal peptide comprises the amino acid sequence of SEQ ID NO: 69 or 70. In some embodiments, the nucleotide sequence encoding a signal peptide comprises SEQ ID NO: 136 or 137.

In some embodiments, a vector encoding a trispecific antibody described herein comprises the nucleotide sequence of SEQ ID NO: 118-120. In some embodiments, a vector encoding a trispecific antibody described herein comprises the nucleotide sequence of SEQ ID NO: 121-123. In some embodiments, a vector encoding a trispecific antibody described herein comprises the nucleotide sequence of SEQ ID NO: 124-126. In some embodiments, a vector encoding a trispecific antibody described herein comprises the nucleotide sequence of SEQ ID NO: 127-129. In some embodiments, a vector encoding a trispecific antibody described herein comprises the nucleotide sequence of SEQ ID NO: 130-132. In some embodiments, a vector encoding a trispecific antibody described herein comprises the nucleotide sequence of SEQ ID NO: 133-135. In some embodiments, the polynucleotides encoding a bispecific antibody described herein further encode a nucleotide sequence encoding a signal peptide. In some embodiments, the signal peptide comprises the amino acid sequence of SEQ ID NO: 69 or 70. In some embodiments, the nucleotide sequence encoding a signal peptide comprises SEQ ID NO: 136 or 137.

In one embodiment, the host cell is selected from the group consisting of E. coli, Pseudomonas, Bacillus, Streptomyces, yeast, CHO, YB/20, NS0, PER-C6, HEK-293T, NIH-3T3, Helga, BHK, Hep G2, SP2/0, R1.1, B-W, L-M, COS 1, COS 7, BSC1, BSC40, BMT10 cell, plant cell, insect cell, and human cell in tissue culture. In one embodiment, the host cell is CHO.

In certain aspects, provided herein is a method of producing an antibody that binds to HIV comprising culturing a host cell described herein so that the polynucleotide is expressed and the antibody is produced. In one embodiment, the method further comprises recovering the antibody.

The isolated polypeptides, i.e., anti-HIV Env antibodies described herein can be produced by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g., Zoeller et al., Proc. Nat'l. Acad. Sci. USA 81:5662-5066 (1984) and U.S. Pat. No. 4,588,585.

In some embodiments a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis or another method), the polynucleotide sequences encoding a particular isolated polypeptide of interest will be inserted into an expression vector and operatively linked to an expression control sequence appropriate for expression of the protein in a desired host. Proper assembly can be confirmed by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding antibodies or fragments thereof. Recombinant expression vectors are replicable DNA constructs which have synthetic or cDNA-derived DNA fragments encoding a polypeptide chain of an antibody or fragment thereof operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of expression control sequence and expression vector will depend upon the choice of host. A variety of host-expression vector systems can be utilized to express antibody molecules described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6. VERO, CRL7030, HsS78Bst, Helga, and NIH 3T3, HEK-293T. HepG2. SP210. R1.1. B-W. L-M. BSC1, BSC40. YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, cells for expressing antibodies described herein are CHO cells, for example CHO cells from the CHO GS System™ (Lonza). In a particular embodiment, cells for expressing antibodies described herein are human cells, e.g., human cell lines. In a specific embodiment, a mammalian expression vector is pOptiVEC™ or pcDNA3.3. In a particular embodiment, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45: 101-105; and Cockett M I et al., (1990) Biotechnology 8: 662-667). In certain embodiments, antibodies described herein are produced by CHO cells or NS0 cells. In a specific embodiment, the expression of nucleotide sequences encoding antibodies described herein which immunospecifically bind Env is regulated by a constitutive promoter, inducible promoter or tissue specific promoter.

For applications where it is desired that the antibodies described herein be expressed in vivo, for example in a subject in need of treatment with an antibody described herein, any vector that allows for the expression of the antibodies and is safe for use in vivo may be used. In one embodiment, the vector is a viral vector. Viral vectors can include poxvirus (vaccinia), including vaccinia Ankara and canarypox; adenoviruses, including adenovirus type 5 (Ad5); rubella; sendai virus; rhabdovirus; alphaviruses; and adeno-associated viruses. In one embodiment, the viral vector is an adeno-associated virus. Alternatively, a polynucleotide encoding the antibody could be delivered as DNA or RNA to the subject for in vivo expression of the antibody.

Suitable host cells for expression of a polypeptide of interest such as an antibody described herein include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are described by Pouwels et al. (Cloning Vectors: A Laboratory Manual, Elsevier, N.Y., 1985), the relevant disclosure of which is hereby incorporated by reference. Additional information regarding methods of protein production, including antibody production, can be found, e.g., in U.S. Patent Publication No. 2008/0187954, U.S. Pat. Nos. 6,413,746 and 6,660,501, and International Patent Publication No. WO 04009823, each of which is hereby incorporated by reference herein in its entirety.

Various mammalian or insect cell culture systems are also advantageously employed to express a recombinant protein such as an antibody described herein. Expression of recombinant proteins in mammalian cells can be performed because such proteins are generally correctly folded, appropriately modified and completely functional. Examples of suitable mammalian host cell lines include but are not limited to CHO, VERO, BHK, Hela, MDCK, HEK 293, NIH 3T3, W138, BT483, Hs578T, HTB2, BT20 and T47D, NS0 (a murine mycloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1. BSC40, YB/20, BMT10 and HsS78Bst cells. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein, e.g., an antibody, into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further an agent. Some or all of the foregoing purification steps, in various combinations, can also be employed to provide a homogeneous recombinant protein.

Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425, 2008/0177048, and 2009/0187005, each of which is hereby incorporated by reference herein in its entirety.

In specific embodiments, an antibody described herein is isolated or purified. Generally, an isolated antibody is one that is substantially free of other antibodies with different antigenic specificities than the isolated antibody. For example, in a particular embodiment, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody. When the polypeptide (e.g., antibody described herein) is recombinantly produced, it is also generally substantially free of culture medium, i.e., culture medium represents less than about 20%, 10%, 2%, 1%, 0.5%, or 0.1% of the volume of the protein preparation. When the polypeptide (e.g., antibody described herein) is produced by chemical synthesis, it is generally substantially free of chemical precursors or other chemicals, i.e., it is separated from chemical precursors or other chemicals which are involved in the synthesis of the protein. Accordingly, such preparations of the polypeptide (e.g., antibody described herein) have less than about 30%, 20%, 10%, or 5% (by dry weight) of chemical precursors or compounds other than the polypeptide of interest. In one embodiment, antibodies described herein are isolated or purified.

### V. Pharmaceutical Compositions

Compositions comprising the multispecific antibodies described herein (e.g., BiS1, BiS 2, BiS 3, BiS 4, BiS 5, BiS 6, BiS 7, BiS 8, BiS 9, BiS 10, BiS 11, BiS 12, BiS 13, BiS 14, TriS 1, TriS 2, TriS 3, TriS 4, TriS 5 or TriS 6) are also provided. Further provided herein are compositions comprising a polynucleotide or polynucleotides encoding the multispecific antibodies described herein. In some embodiments, the polynucleotide comprises mRNA. In some embodiments, the composition is a pharmaceutical composition. In some embodiment, the multispecific antibody is BiS13 or BiS14. In some embodiment, the multispecific antibody is BiS13.

In some embodiments, the composition is a lyophilized composition.

In certain aspects, provided herein is a pharmaceutical composition comprising a multispecific antibody described herein (e.g., BiS1, BiS 2, BiS 3, BiS 4, BiS 5. BiS 6, BiS 7, BiS 8, BiS 9, BiS 10, BiS 11, BiS 12, BiS 13, BiS 14, TriS 1, TriS 2, TriS 3, TriS 4, TriS 5 or TriS 6) and a pharmaceutically acceptable excipient. Such compositions are intended, for example, for prevention and treatment of HIV infection. In some embodiments, the composition is formulated for intravenous administration. In some embodiments, the composition is formulated for intramuscular administration. In some embodiments, the composition is formulated for subcutaneous administration. In some embodiments, the composition is formulated for topical administration, and in certain embodiments the composition is formulated for vaginal or rectal administration.

In further embodiments of the present disclosure, a composition comprising the multispecific antibody described herein can additionally be combined with other compositions for the treatment of HIV infection or the prevention of HIV transmission.

In some embodiments, a multispecific antibody described herein may be administered within a pharmaceutically-acceptable diluent, carrier, or excipient, in unit dose form. Conventional pharmaceutical practice may be employed to provide suitable formulations or compositions to administer to individuals being treated for HIV infection. In some embodiments, the administration is prophylactic. Any appropriate route of administration may be employed, for example, administration may be parenteral, intravenous, intra-arterial, subcutaneous, intramuscular, intraperitoneal, intranasal, aerosol, suppository, oral administration, vaginal, or anal.

The pharmaceutical compositions described herein are prepared in a manner known per se, for example, by means of conventional dissolving, lyophilizing, mixing, granulating or confectioning processes. The pharmaceutical compositions may be formulated according to conventional pharmaceutical practice (see for example, in Remington: The Science and Practice of Pharmacy (21st ed.), ed. A. R. Gennaro, 2005, Lippincott Williams & Wilkins, Philadelphia, PA, and Encyclopedia of Pharmaceutical Technology, eds. J. Swarbrick and J. C. Boylan, 2013, Marcel Dekker, New York, NY).

The injection compositions are prepared in customary manner under sterile conditions; the same applies also to introducing the compositions into ampoules or vials and sealing the containers.

Pharmaceutical compositions according to the invention may be, for example, in unit dose form, such as in the form of ampoules, vials, suppositories, tablets, pills, or capsules. The formulations can be administered to human individuals in therapeutically or prophylactic effective amounts (e.g., amounts which prevent, eliminate, or reduce a pathological condition) to provide therapy for a disease or condition. The preferred dosage of therapeutic agent to be administered is likely to depend on such variables as the type and extent of the disorder, the overall health status of the particular patient, the formulation of the compound excipients, and its route of administration.

In certain embodiments, the compositions described herein can be formulated for topical administration, and in certain embodiments the composition is formulated for vaginal or rectal administration. The composition may be formulated as a gel, or formulated as a topical cream, ointment, lotion or foam formulation. Useful formulations are known in the art, for example, as disclosed in U.S. Patent Appl. Pub. No. 20130022619, which is incorporated by reference herein in its entirety for all purposes.

In certain embodiments, the composition may further comprise a pharmaceutically acceptable excipient, a lubricant, or an antiviral agent.

The topical formulations of the present invention can be used to prevent HIV infection in a human, or to inhibit transmission of the HIV virus from an infected human to another human. The topical formulations of the present invention can inhibit the growth or replication of HIV. The topical formulations are useful in the prophylactic treatment of humans who are at risk for HIV infection. The topical formulations also can be used to treat objects or materials, such as contraceptive devices (for example condoms or intrauterine devices), medical equipment, supplies, or fluids, including biological fluids, such as blood, blood products, and tissues, to prevent or inhibit viral infection of a human. Such topical formulations also are useful to prevent transmission, such as sexual transmission of viral infections, e.g., HIV, which is the primary way in which HIV is transmitted globally. The methods of prevention or inhibition or retardation of transmission of viral infection, e.g., HIV infection, in accordance with the present invention, comprise vaginal, rectal, penile or other topical treatment with an antiviral effective amount of a topical preparation of the present invention, alone or in combination with another antiviral compound as described herein.

In some embodiments the composition is in the form of a cream, lotion, gel, or foam that is applied to the affected skin or epithelial cavity, and preferably spread over the entire skin or epithelial surface which is at risk of contact with bodily fluids. Such formulations, which are suitable for vaginal or rectal administration, may be present as aqueous or oily suspensions, solutions or emulsions (liquid formulations) containing in addition to the active ingredient, such carriers as are known in the art to be appropriate. These formulations are useful to protect not only against sexual transmission of HIV, but also to prevent infection of a baby during passage through the birth canal. Thus the vaginal administration can take place prior to sexual intercourse, during sexual intercourse, and immediately prior to childbirth.

As a vaginal formulation, the active ingredient may be used in conjunction with a spermicide and may be employed with a condom, diaphragm, sponge or other contraceptive device. Examples of suitable spermicides include nonylphenoxypolyoxyethylene glycol (nonoxynol 9), benzethonium chloride, and chlorindanol. Suitably, the pH of the composition is 4.5 to 8.5. Vaginal compositions preferably have a pH of 4.5 to 6, most preferably about 5.

Vaginal formulations include suppositories (for example, gel-covered creams), tablets and films. The suppositories can be administered by insertion with an applicator using methods well known in the art.

Vaginal formulations further include vaginal ring devices formulated for sustained release. See, e.g., Morrow et al., Eur J Pharm Biopharm. 77(1):3-10 (2011), Zhao et al., Antimicrob Agents Chemother. 61(7) pii: e02465-16 (2017).

Buccal formulations include creams, ointments, gels, tablets or films that comprise ingredients that are safe when administered via the mouth cavity. Buccal formulations can also comprise a taste-masking or flavoring agent.

The present compositions may be associated with a contraceptive device or article, such as a vaginal ring device, an intrauterine device (IUD), vaginal diaphragm, vaginal sponge, pessary, condom, etc.

In some embodiments the compositions described herein are used in conjunction with condoms, to enhance the risk-reducing effectiveness of condoms and provide maximum protection for users. The composition can either be coated onto condoms during manufacture, and enclosed within conventional watertight plastic or foil packages that contain one condom per package, or it can be manually applied by a user to either the inside or the outside of a condom, immediately before use. As used herein, “condom” refers to a barrier device which is used to provide a watertight physical barrier between male and female genitalia during sexual intercourse, and which is removed after intercourse. This term includes conventional condoms that cover the penis; it also includes so-called “female condoms” which are inserted into the vaginal cavity prior to intercourse.

In another embodiment a composition described herein is in the form of an intra-vaginal pill, an intra-rectal pill, or a suppository. The suppository or pill should be inserted into the vaginal or rectal cavity in a manner that permits the suppository or pill, as it dissolves or erodes, to coat the vaginal or rectal walls with a prophylactic layer of a multispecific antibody described herein.

In certain embodiments, the composition may further comprise a pharmaceutically acceptable excipient, a lubricant, or an antiviral agent.

Compositions used in the methods of this invention may also comprise other active agents, such as another agent to prevent HIV infection, and agents that protect individuals from conception and other sexually transmitted diseases. Thus, in another embodiment the compositions used in this invention further comprise a second anti-HIV agent, a virucide effective against viral infections other than HIV, and/or a spermicide.

The compositions used in this invention may also contain a lubricant that facilitates application of the composition to the desired areas of skin and epithelial tissue, and reduces friction during sexual intercourse. In the case of a pill or suppository, the lubricant can be applied to the exterior of the dosage form to facilitate insertion.

In the cream or ointment embodiments of the present invention, the topical formulation comprises one or more lubricants. The gels and foams of the present invention optionally can include one or more lubricants.

Non-limiting examples of useful lubricants include cetyl esters wax, hydrogenated vegetable oil, magnesium stearate, methyl stearate, mineral oil, polyoxyethylene-polyoxypropylene copolymer, polyethylene glycol, polyvinyl alcohol, sodium lauryl sulfate, white wax, or mixtures of two or more of the above.

The gel formulations of the present invention comprise one or more gelling agents. Non-limiting examples of useful gelling agents include carboxylic acid polymers including acrylic acid polymers crosslinked with cross links such as allyl ethers of sucrose (e.g. carbomer brand thickeners), cetostearyl alcohol, hydroxymethyl cellulose, polyoxyethylene-polyoxypropylene copolymer, sodium carboxymethylcellulose, polyvinyl pyrrolidone, or mixtures of two or more thereof.

### VI. Therapeutic Uses and Methods

In one aspect, provided herein is a method of treating HIV or inhibiting transmission of HIV. In one embodiment, the method of inhibiting transmission of HIV comprises administering to a subject in need thereof an effective amount of an multispecific antibody described herein (e.g., BiS1, BiS 2. BiS 3, BiS 4. BiS 5, BiS 6. BiS 7, BiS 8. BiS 9, BiS 10, BiS 11, BiS 12, BiS 13, BiS 14, TriS 1, TriS 2, TriS 3, TriS 4, TriS 5 or TriS 6), a pharmaceutical composition described herein, an isolated polynucleotide described herein, or a recombinant virus (e.g., recombinant AAV) described herein. In one embodiment, the method of inhibiting transmission of HIV comprises administering to a subject in need thereof an effective amount of a multispecific antibody described herein. In one embodiment, the method of inhibiting transmission of HIV comprises administering to a subject in need thereof an effective amount of a recombinant AAV encoding a multispecific antibody described herein. In one embodiment, the method of inhibiting transmission of HIV comprises administering to a subject in need thereof an effective amount of an multispecific antibody described herein. In one embodiment, the subject has been exposed to HIV. In one embodiment, the subject is at risk of being exposed to HIV. In one embodiment, the subject at risk of being exposed to HIV is a health care worker, a sexual partner of an HIV infected individual, or a sex worker. In one embodiment, the subject that has been exposed to HIV or is at risk of being exposed to HIV is a newborn. In some embodiment, the multispecific antibody is BiS13 or BiS14. In some embodiment, the multispecific antibody is BiS13.

In one aspect, provided herein is a method of reducing the risk of a subject becoming infected with HIV comprising administering to the subject in need thereof an effective amount of a multispecific antibody described herein (e.g., BiS1, BiS 2, BiS 3, BiS 4, BiS 5, BiS 6, BiS 7, BiS 8, BiS 9, BiS 10, BiS 11, BiS 12, BiS 13, BiS 14, TriS 1, TriS 2, TriS 3, TriS 4, TriS 5 or TriS 6), a pharmaceutical composition described herein, an isolated polynucleotide described herein, or a recombinant virus described herein. In one embodiment, the method comprises administering to a subject in need thereof an effective amount of a multispecific antibody described herein. In one embodiment, the method comprises administering to a subject in need thereof an effective amount of a recombinant AAV encoding a multispecific antibody described herein. In one embodiment, the subject has been exposed to HIV. In one embodiment, the subject is at risk of being exposed to HIV. In one embodiment, the subject at risk of being exposed to HIV is a health care worker, a sexual partner of an HIV infected individual, or a sex worker. In one embodiment, the subject that has been exposed to HIV or is at risk of being exposed to HIV is a newborn. In one aspect, provided herein is a multispecific antibody, a pharmaceutical composition, an isolated polynucleotide, or a recombinant virus for reducing the risk of a subject becoming infected with HIV. In some embodiment, the multispecific antibody is BiS13 or BiS14. In some embodiment, the multispecific antibody is BiS13.

In one aspect, provided herein is a method for passively immunizing a subject comprising administering to the subject in need thereof an effective amount of an multispecific antibody described herein (e.g., BiS1, BiS 2, BiS 3, BiS 4, BiS 5, BiS 6, BiS 7, BiS 8, BiS 9, BiS 10, BiS 11, BiS 12, BiS 13, BiS 14, TriS 1, TriS 2, TriS 3, TriS 4, TriS 5 or TriS 6), a pharmaceutical composition described herein, an isolated polynucleotide described herein, or a recombinant virus described herein. In one embodiment, the method comprises administering to a subject in need thereof an effective amount of a multispecific antibody described herein. In one embodiment, the method comprises administering to a subject in need thereof an effective amount of a recombinant AAV encoding a multispecific antibody described herein. In one embodiment, the subject has been exposed to HIV. In one embodiment, the subject is at risk of being exposed to HIV. In one embodiment, the subject at risk of being exposed to HIV is a health care worker, a sexual partner of an HIV infected individual, or a sex worker. In one embodiment, the subject that has been exposed to HIV or is at risk of being exposed to HIV is a newborn. In one aspect, provided herein is provided herein is a multispecific, a pharmaceutical composition, an isolated polynucleotide, or a recombinant virus for passively immunizing a subject. In some embodiment, the multispecific antibody is BiS13 or BiS14. In some embodiment, the multispecific antibody is BiS13.

Further provided herein is a method of neutralizing an HIV virus comprising contacting the virus with an effective amount of a multispecific antibody described herein (e.g., BiS1, BiS 2, BiS 3. BiS 4, BiS 5, BiS 6, BiS 7, BiS 8. BiS 9, BiS 10, BiS 11, BiS 12, BiS 13, BiS 14, TriS 1, TriS 2, TriS 3, TriS 4, TriS 5 or TriS 6). In one embodiment, the virus is comprised by a composition, for example, a fluid, including a biological fluid, such as blood or blood product. In certain embodiments, the method comprises adding a multispecific antibody described herein to a composition comprising HIV in a sufficient amount or concentration to neutralize the HIV.

Further provided herein is a method of reducing viral load comprising administering to a subject in need thereof an effective amount of a multispecific antibody (e.g., BiS1, BiS 2, BiS 3, BiS 4, BiS 5, BiS 6, BiS 7, BiS 8, BiS 9, BiS 10, BiS 11, BiS 12, BiS 13, BiS 14, TriS 1, TriS 2, TriS 3, TriS 4, TriS 5 or TriS 6), a pharmaceutical composition described herein, an isolated polynucleotide described herein, or a recombinant virus described herein. In one embodiment, the method comprises administering to a subject in need thereof an effective amount of a multispecific antibody described herein. In one embodiment, the method comprises administering to a subject in need thereof an effective amount of a recombinant AAV encoding a multispecific antibody described herein. In some embodiment, the multispecific antibody is BiS13 or BiS14. In some embodiment, the multispecific antibody is BiS13.

In one embodiment, a method of preventing HIV infection provided herein comprises administering to a subject in need thereof a therapeutically sufficient amount of a multispecific antibody described herein (e.g., BiS1, BiS 2, BiS 3. BiS 4, BiS 5. BiS 6, BiS 7, BiS 8, BiS 9. BiS 10, BiS 11, BiS 12, BiS 13, BiS 14, TriS 1, TriS 2, TriS 3, TriS 4, TriS 5 or TriS 6), a pharmaceutical composition described herein, an isolated polynucleotide described herein, or a recombinant virus described herein. In some embodiment, the multispecific antibody is BiS13 or BiS14. In some embodiment, the multispecific antibody is BiS13.

In one embodiment, a method of treating HIV/AIDS provided herein comprises administering to a subject in need thereof a therapeutically sufficient amount of a multispecific antibody described herein (e.g., BiS1, BiS 2, BiS 3, BiS 4, BiS 5, BiS 6, BiS 7, BiS 8, BiS 9, BiS 10, BiS 11, BiS 12, BiS 13, BiS 14, TriS 1, TriS 2, TriS 3, TriS 4, TriS 5 or TriS 6), a pharmaceutical composition described herein, an isolated polynucleotide described herein, or a recombinant virus described herein. In one embodiment, a method of treating HIV/AIDS comprises administering a multispecific antibody described herein. In one embodiment, a method of treating HIV/AIDS comprises administering a pharmaceutical composition described herein. In one embodiment, a method of treating HIV/AIDS comprises administering an isolated polynucleotide described herein. In one embodiment, a method of treating HIV/AIDS comprises administering a recombinant virus described herein. In one aspect, provided herein is an antibody, a pharmaceutical composition, an isolated polynucleotide, or a recombinant virus for treating HIV/AIDS. In some embodiment, the multispecific antibody is BiS13 or BiS14. In some embodiment, the multispecific antibody is BiS13.

In one embodiment, the administering to the subject is by at least one mode selected from oral, parenteral, subcutaneous, intramuscular, intravenous, vaginal, rectal, buccal, sublingual, and transdermal

In one embodiment, a method of treatment described herein further comprises administering at least one additional therapeutic agent. In one embodiment, the additional therapeutic agent comprises an antiretroviral therapy (ART) agent, a reservoir activator, an immunomodulator, a second antibody, or a second and third antibody. In one embodiment, the additional therapeutic agent comprises a second antibody. In one embodiment, the additional therapeutic agent comprises a second and third antibody. In one embodiment, the additional therapeutic agent comprises a second and optionally third antibody which is an anti-HIV antibody. In one embodiment, the additional therapeutic agent comprises a second and optionally third antibody which is an anti-HIV Env antibody. In one embodiment, the additional therapeutic agent comprises a second and optionally third anti-HIV Env antibody which binds to an HIV Env epitope region different from the HIV Env epitope region bound by a multispecific antibody disclosed herein. In one embodiment, the additional therapeutic agent comprises a second and optionally third anti-HIV Env antibody which binds to the gp120-gp41 interface epitope region or the gp41 membrane proximal external epitope region (MPER). In one embodiment, the additional therapeutic agent comprises a second anti-HIV Env antibody which binds to the gp120-gp41 interface epitope region. In one embodiment, the additional therapeutic agent comprises a second anti-HIV Env antibody which binds to the gp41 membrane proximal external epitope region (MPER).

In certain embodiments, the subject is at risk for exposure to HIV. In some embodiments, the subject is infected with HIV. In some embodiments, the subject is diagnosed with AIDS. In certain embodiments, the subject at risk for exposure to HIV is a health care worker. In certain embodiments, the subject at risk for exposure to HIV is a sex worker. In certain embodiments, the subject at risk for exposure to HIV is a sexual partner of an HIV infected individual. In certain embodiments, the subject at risk for exposure to HIV is a newborn.

The invention also features methods of blocking HIV infection in a subject (e.g., a human) at risk of HIV transmission. For example, in one aspect, the subject may be a fetus of an HIV-infected pregnant female and the method includes administering to the HIV-infected pregnant female a multispecific antibody described herein, thereby blocking the HIV infection in the fetus. In other instances, the subject is a newborn having an HIV-infected mother, a subject at risk of HIV transmission following a needle stick injury, or a subject at risk of HIV transmission following a sexual exposure to an HIV-infected individual.

In instances when the subject is a newborn having an HIV-infected mother, the newborn can be administered a multispecific antibody described herein peripartum and/or postpartum, for example, prior to, during, and/or following breastfeeding from the HIV-infected mother, in order to block an HIV infection in the newborn.

In instances when the subject is at risk of HIV transmission following a sexual exposure to an HIV-infected individual, the subject can be administered a multispecific antibody described herein following the sexual exposure in order to block an HIV infection in the subject.

In some embodiments, an multispecific antibody described herein can be used as a microbicides to prevent mucosal HIV acquisition. In some embodiments, a multispecific antibody described herein is used to prevent vaginal or rectal acquisition of HIV. In some embodiments, an multispecific antibody described herein can be used as a microbicides to reduce the likelihood of mucosal HIV acquisition. In some embodiments, a multispecific antibody described herein is used to reduce the likelihood of vaginal or rectal acquisition of HIV.

In any of the methods described above, further administration of ART and/or an immunomodulator and/or a second antibody is contemplated. For example, the ART and/or immunomodulator and/or a second antibody can be administered in conjunction with, prior to, concurrently with, subsequent to, or within the context of a treatment regimen that includes administration of a multispecific antibody described herein.

A multispecific antibody described herein, or a pharmaceutical composition described herein can be delivered to a subject by a variety of routes, such as oral, parenteral, subcutaneous, intravenous, intradermal, transdermal, intranasal, vaginal, or anal. In one embodiment, the antibody or pharmaceutical composition is administered intravenously, vaginally, or anally.

The amount of a multispecific antibody described herein, or a pharmaceutical composition described herein which will be effective in the treatment and/or prevention of a condition will depend on the nature of the disease, and can be determined by standard clinical techniques.

The precise dose to be employed in a pharmaceutical composition will also depend on the route of administration, and the seriousness of the disease, and should be decided according to the judgment of the practitioner and each subject's circumstances. For example, effective doses may also vary depending upon means of administration, target site, physiological state of the patient (including age, body weight and health), whether the patient is human or an animal, other medications administered, or whether treatment is prophylactic or therapeutic. Usually, the patient is a human but non-human mammals including transgenic mammals can also be treated. Treatment dosages are optimally titrated to optimize safety and efficacy.

In certain embodiments, an in vitro assay is employed to help identify optimal dosage ranges. Effective doses may be extrapolated from dose response curves derived from in vitro or animal model test systems.

### EXAMPLES

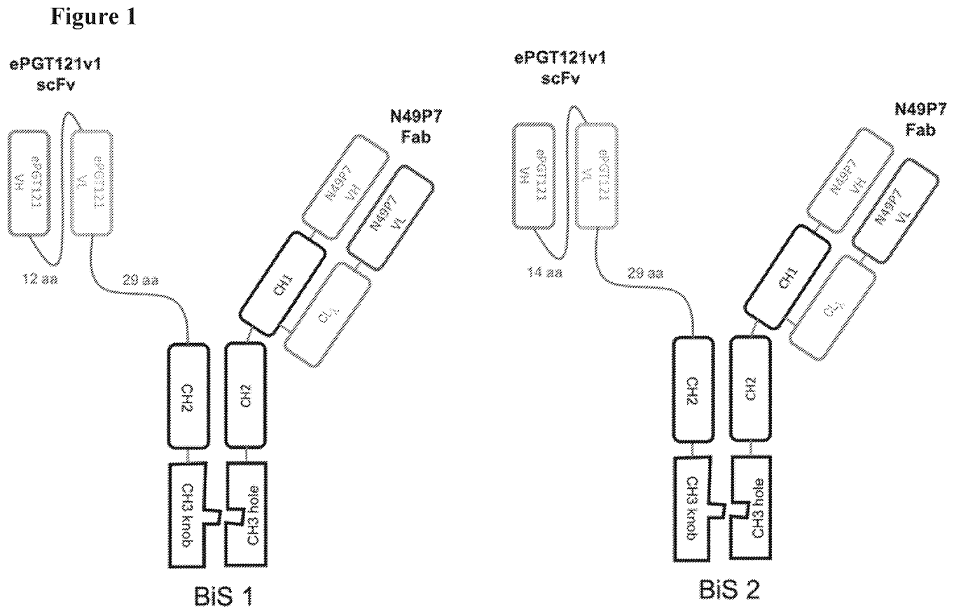

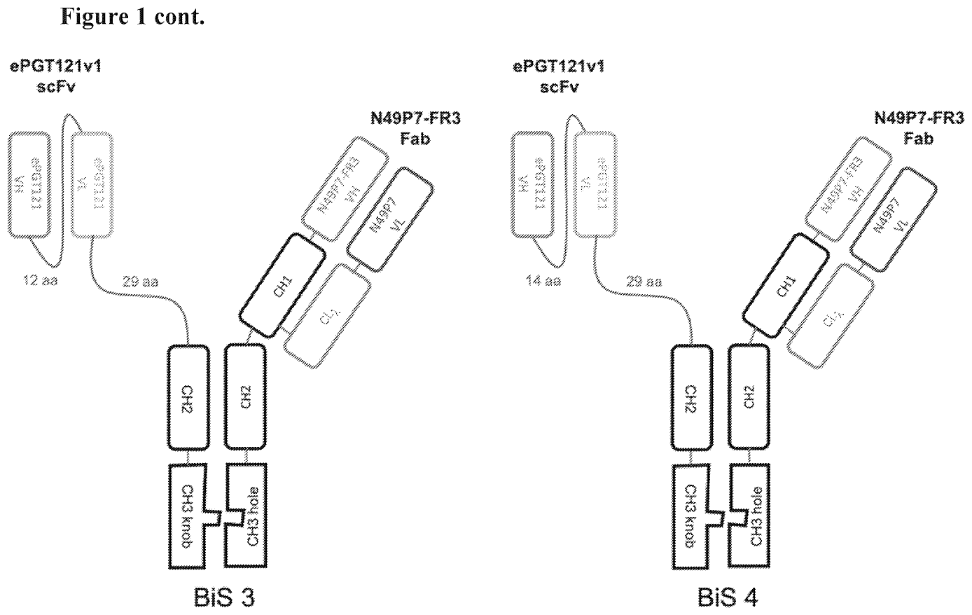

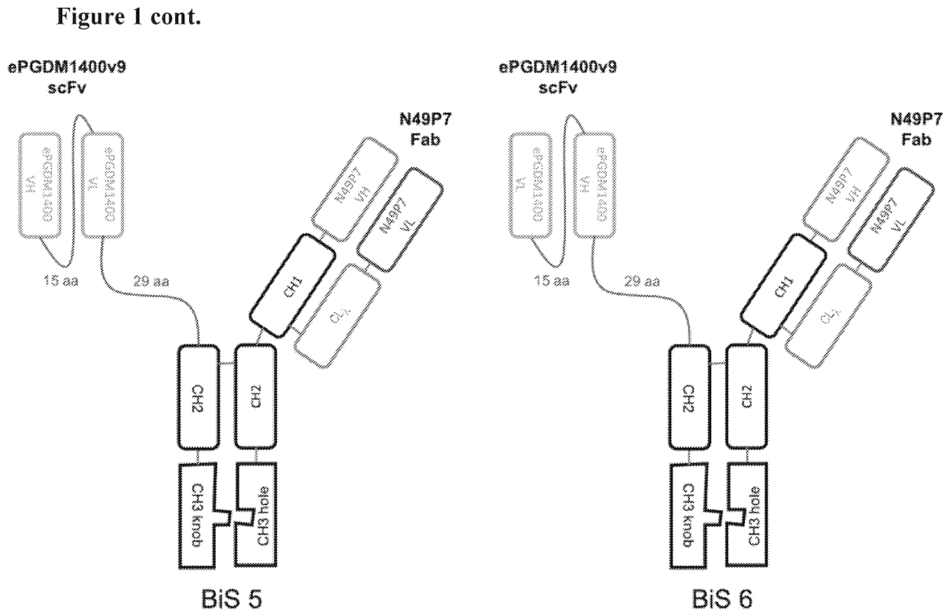

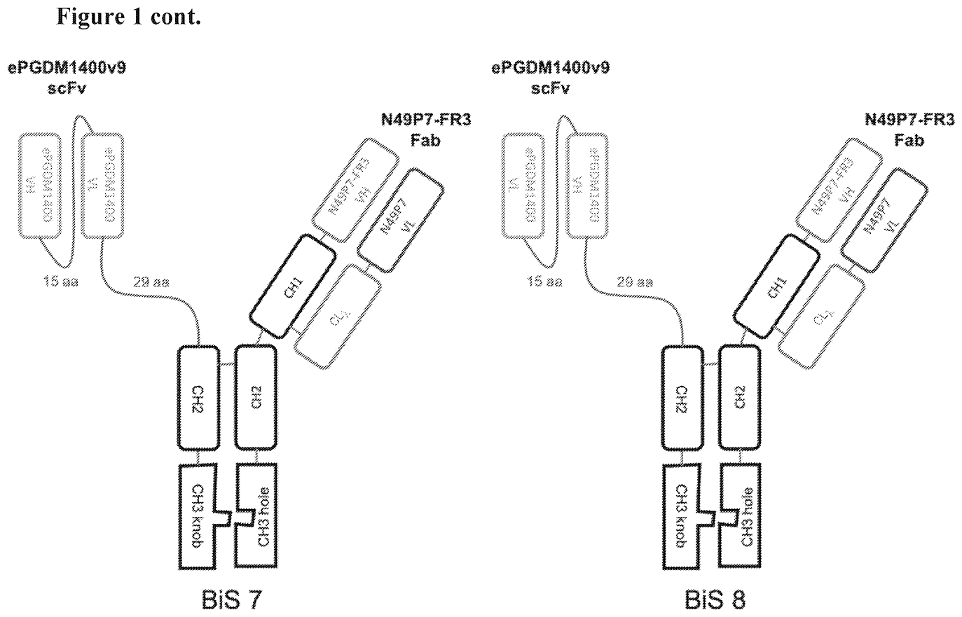

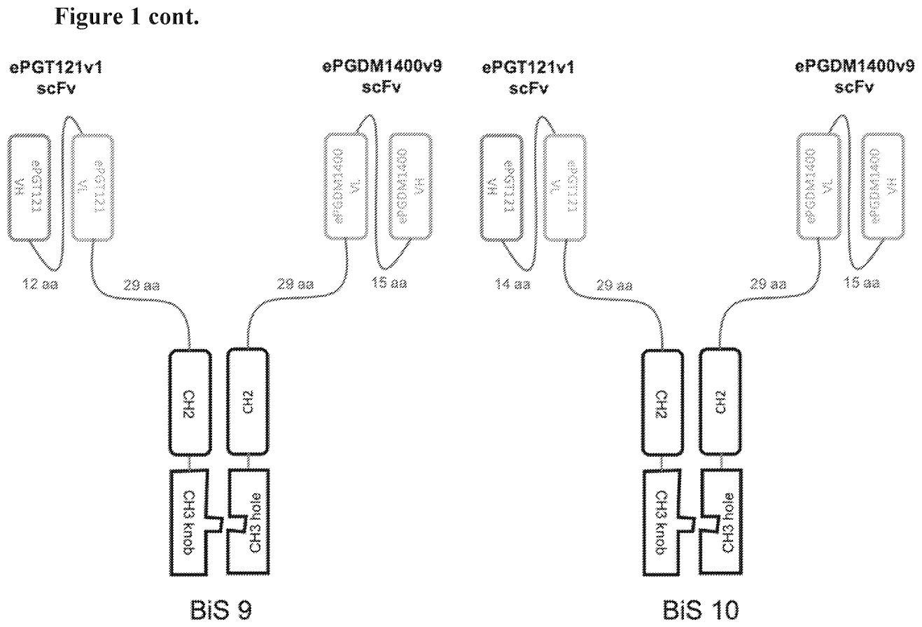

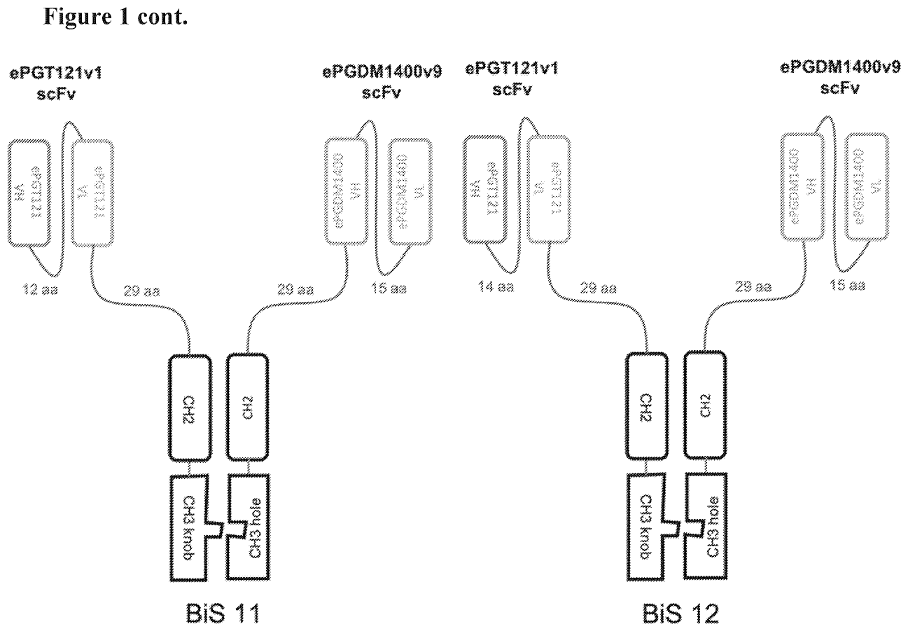

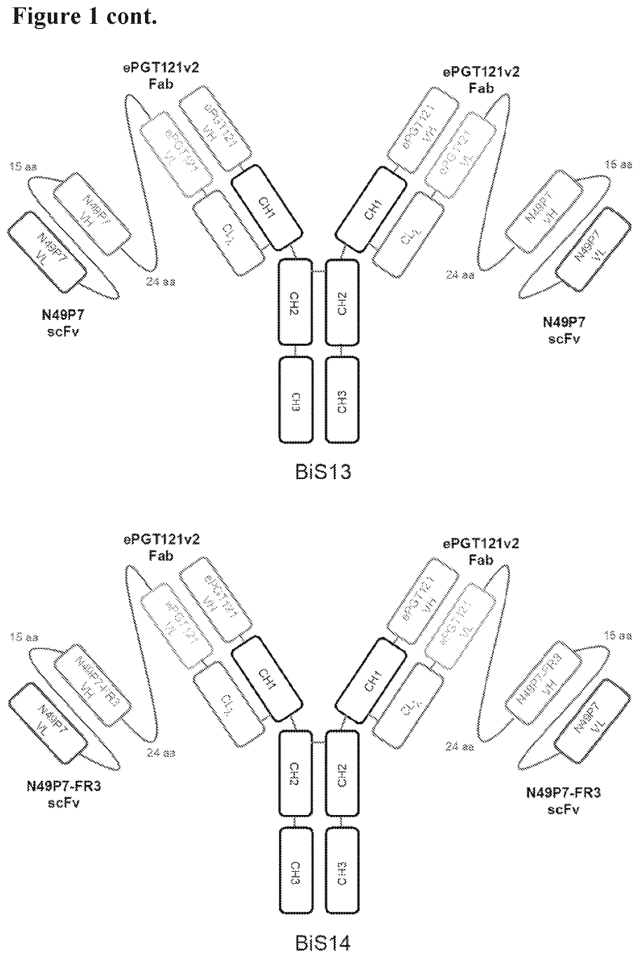

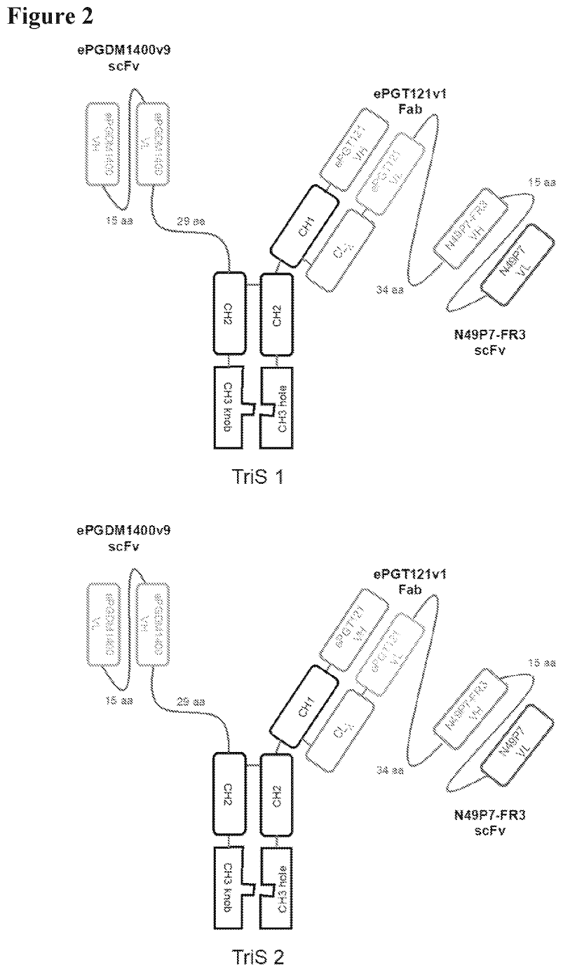

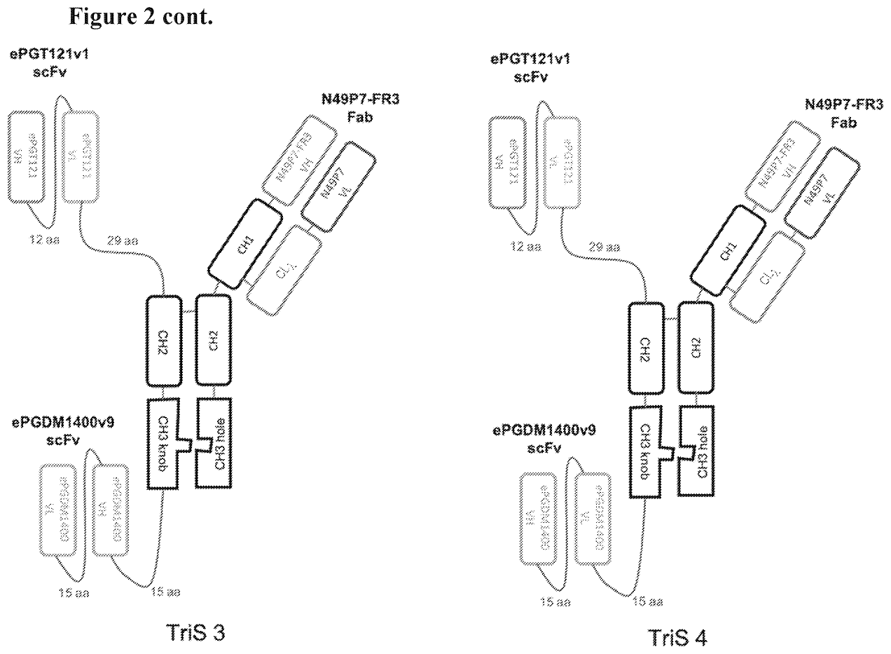

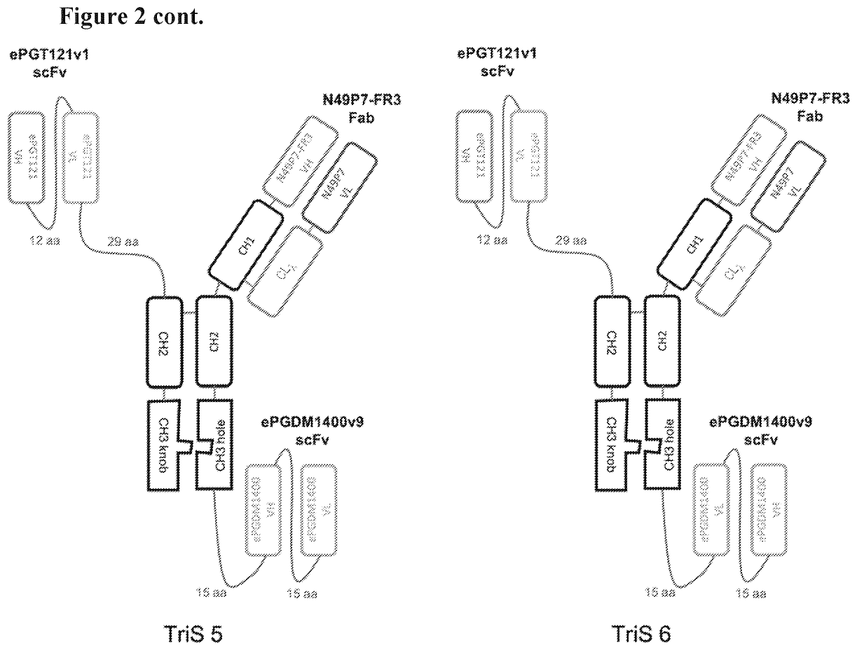

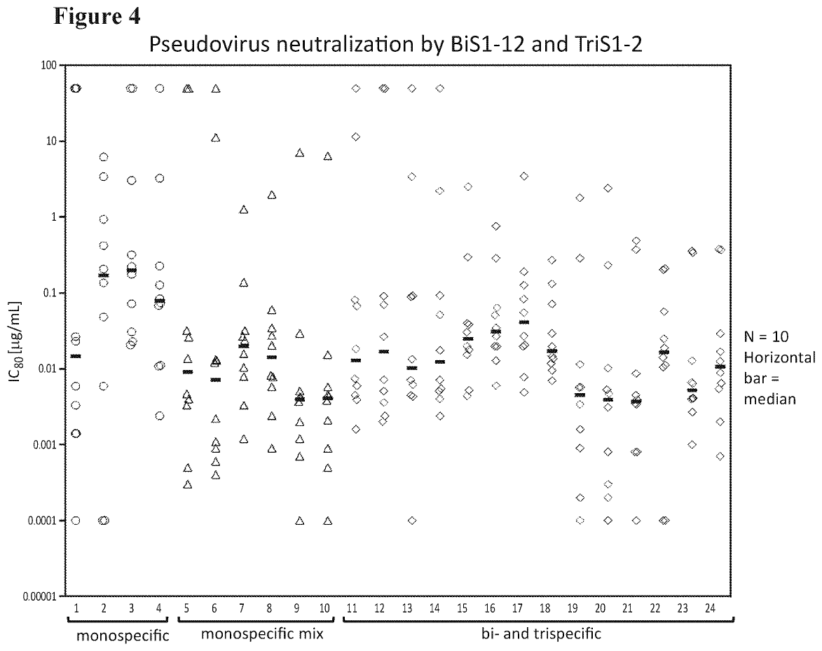

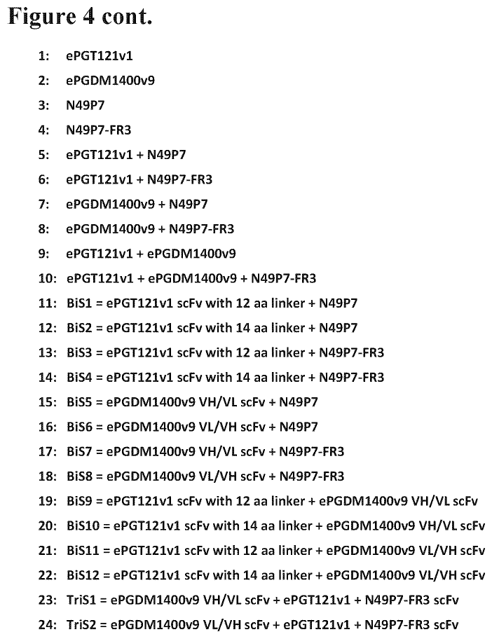

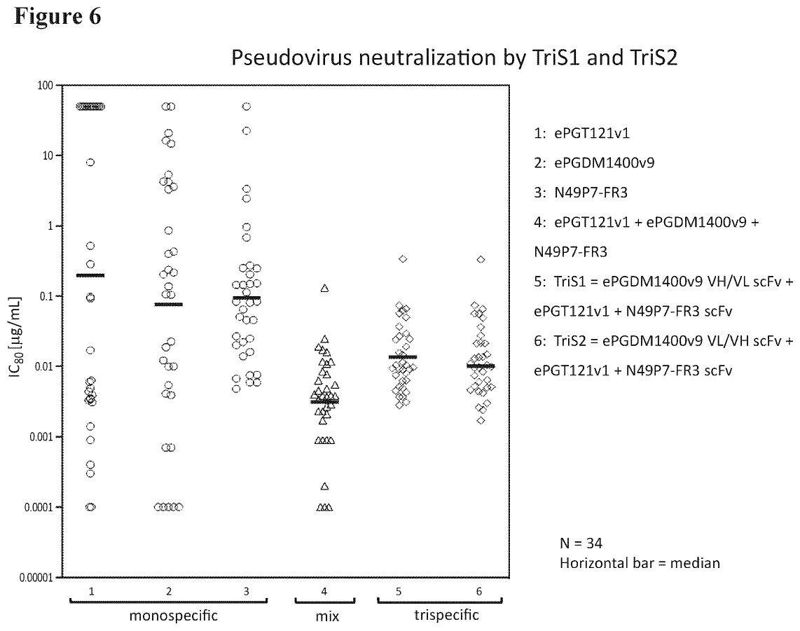

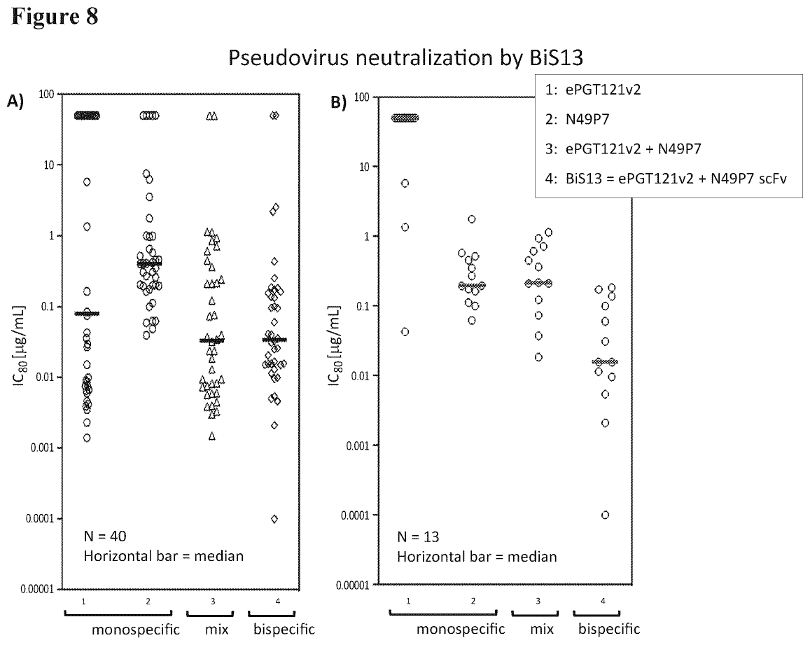

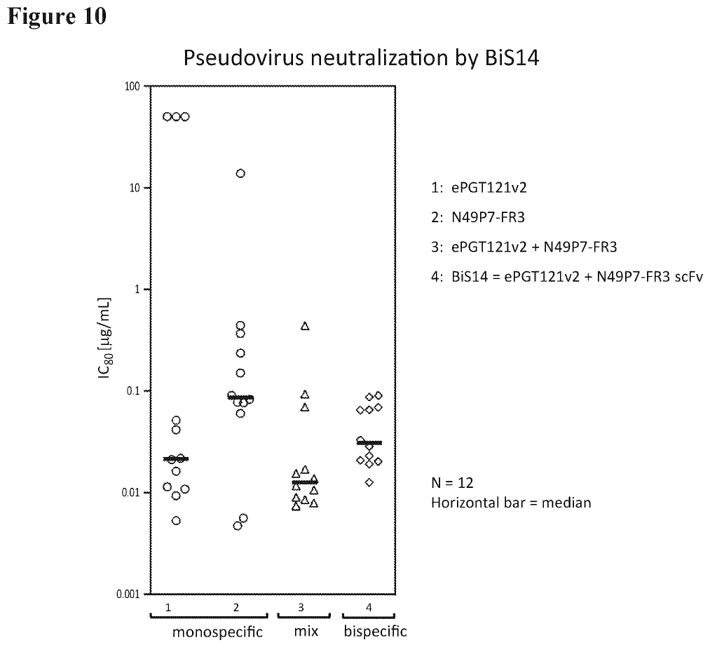

Bi- and tri-specific antibodies shown in FIGS. 1 and 2 were produced as described below. Neutralization activity of the antibodies was tested using an HIV-1 pseudovirus neutralization assay. The results of the assay are shown in FIGS. 3 to 10. Among the bi- and trispecific antibodies tested against a panel of 10 HIV-1 pseudoviruses, BiS9, BiS10, BiS11, TriS1, and TriS2 showed the highest breadth and potency with median IC₈₀ in a range of 0.004-0.011 μg/mL (FIGS. 3 and 4). TriS1 and TriS2 potently neutralized an additional panel of 34 pseudoviruses with median IC₈₀ of 0.013 and 0.010 μg/mL, respectively (FIGS. 5 and 6). BiS13 and the mixture of corresponding monospecific antibodies exhibited similar breadth and potency against 40 pseudoviruses with median IC₈₀ of 0.034 and 0.033 μg/mL, respectively (FIGS. 7 and 8). BiS14 tested against a panel of 12 pseudoviruses showed higher breadth than the corresponding monospecific antibodies alone, and similar breadth but lower potency (median IC₈₀ of 0.031 μg/mL) compared to the mixture of monospecific antibodies (median IC₈₀ of 0.013 μg/mL; FIGS. 9 and 10).

### 1. Antibody Production

NEB 5-alpha competent E. coli cells were transformed with a plasmid DNA encoding an antibody chain, and were grown on LB broth agar, supplemented with carbenicillin (100 μg/mL), at 37° C. for 16-20 hours. A single colony was transferred to Plasmid+ Media supplemented with carbenicillin (100 μg/mL) and grown at 37° C. for 16-20 hours with agitation. The plasmid DNA was isolated using the NucleoSpin® plasmid transfection-grade mini kit.

Expi293F cells were maintained in Expi293 Expression Medium and transfected with the plasmid DNA in a transfection mixture composed of: OptiMEM™, plasmid DNA, FectoPRO® Transfection Reagent, Expi293F™, VPA, and glucose (see Table 3 for details). The cells were incubated at 37° C. in a CO₂ incubator for 5 days. The supernatants containing secreted antibodies were then collected by centrifugation, sterilized through a 0.22 μm filter, and incubated with Praesto® AP agarose resin (supernatant to resin v/v ratio of 50 to 1) at 4° C. for 16-20 hours with gentle rotated mixing. The resin was packed onto a chromatographic column and washed with DPBS using a 10-fold resin volume. Antibodies were eluted from the column with IgG elution buffer into neutralization buffer (elution buffer to neutralization buffer v/v ratio of 10 to 1) and dialyzed for 12-20 hours against DPBS in a 10,000 MWCO dialysis cassette. Antibody concentrations were calculated based on their molecular weight, extinction coefficient, and absorbance measured at 280 nm by a NanoDrop™ One spectrophotometer. Antibody chains were identified by SDS-PAGE upon protein denaturation in a reducing buffer.

TABLE 3
Transfection of Expi293F cells

| Final | | | FectoPRO ® | | | |
|---|---|---|---|---|---|---|
| transfection | | Plasmid | Transfection | | | |
| volume | OptiMEM ™ | DNA¹ | Reagent | Expi293F ™ | VPA³ | Glucose³ |
| [mL] | [mL] | [μg] | [μL] | cells² [mL] | [mL] | [mL] |
| 30 | 3 | 24 | 24 | 27 | 0.3 | 0.27 |
| 50 | 5 | 40 | 40 | 45 | 0.5 | 0.45 |
| 100 | 10 | 80 | 80 | 90 | 1 | 0.9 |
| 200 | 20 | 160 | 160 | 180 | 2 | 1.8 |
| 500 | 50 | 400 | 400 | 450 | 5 | 4.5 |
| 1000 | 100 | 800 | 800 | 900 | 10 | 9 |

¹Plasmid DNA was used at a mass ratio of heavy chain A:heavy chain B:light chain = 1:1:2 (BiS1-8, TriS1-6), heavy chain A:heavy chain B = 1:1 (BiS9-12), or heavy chain:light chain = 1:2 (BiS13, BiS14).
²Expi293F cells were used at the concentration of 3 × 10⁶ cells per milliliter.
³Expi293F were fed with VPA and glucose 20-24 hours after transfection.

### 2. HIV-1 Pseudovirus Neutralization Assay

An HIV-1 neutralization assay was performed by measuring the reduction in luciferase expression following a single round of virus infection in TZM-bl cells. All produced antibodies were tested against a panel of HIV-1 pseudoviruses belonging to the Seaman panel and the Montefiori (global) panel. Pseudoviruses were generated by cotransfecting 293T cells with an envelope-expressing plasmid and a backbone plasmid pSG3ΔENV that has an envelope-defective HIV-1 genome, using PEI as a transfection reagent. Virus supernatants were harvested 72 h after transfection and filtered through a 0.45 μm membrane. To measure the antiviral activity of antibodies, they were prepared in 10-fold dilutions (final concentrations ranging from 50 to 0.0005 μg/mL) in DMEM in duplicate, mixed with an equal volume of pseudovirus supernatants in a 96-well plate, supplemented with DEAE-dextran at a final concentration of 10 μg/mL, and incubated 1 h at 37° C. in a CO₂ incubator. TZM-bl cells in DMEM were added to the mixture (10⁴/well) and incubated 48-72 h at 37° C. in a CO₂ incubator. Assay controls included replicate wells of TZM-bl cells alone (cell control) and TZM-bl cells with virus (virus control). Luciferase activity was measured using luciferase assay reagents and a luminescence mode of a microplate reader when cells reached a luminescence value that was at least 50 times higher for virus control compared to cell control. Percent inhibition of the pseudovirus by an antibody and 80% inhibitory concentration (IC₈₀) were calculated using GraphPad Prism software.

FIGS. 7 and 8A shows the neutralization breadth and potency of BiS13 and its parental monoclonal antibodies used alone or in combination against a panel of 40 HIV-1 pseudoviruses. Table 4 shows extracted results from FIG. 7 generated with a subset of 13 pseudoviruses that are mostly resistant to ePGT121 and moderately sensitive to N49P7. FIG. 8B shows the neutralization potency of BiS13 and its parental monoclonal antibodies against the same subset of 13 pseudoviruses. For this specific subset of 13 pseudoviruses, BiS13 showed synergistically increased neutralization potency compared to the parental antibodies used in combination (compare FIG. 8B lanes 3 and 4, demonstrating over 13-fold improved median IC₈₀). This result suggests the bivalent binding of BiS13 on the HIV-1 Env trimer, resulting in increased antibody avidity.

TABLE 4
Extract from FIG. 7: Pseudovirus neutralization by BiS13 (IC₈₀ [μg/mL]).

| | | | | ePGT121 + | |
|---|---|---|---|---|---|
| HIV pseudovirus | Clade | ePGT121 | N49P7 | N49P7 | BiS13 |
| Q259.17 | A | >50 | 0.1625 | 0.1225 | 0.0114 |
| MS208.A1 | A | >50 | 0.1944 | 0.2096 | 0.0998 |
| 6545.V4.C1 | AC | >50 | 1.7620 | 1.1390 | <0.0001 |
| BJOX009000.02.4 | AE | 0.0428 | 0.0999 | 0.0184 | 0.0096 |
| CNE8 | AE | >50 | 0.1750 | 0.2108 | 0.1372 |
| BJOX010000.06.2 | AE (T/F) | >50 | 0.4531 | 0.4495 | 0.1723 |
| T257-31 | AG | >50 | 0.5772 | 0.7165 | 0.1828 |
| QH0692.42-N332A | B | >50 | 0.3499 | 0.3636 | 0.0311 |
| TRO.11-N332A | B | 1.3520 | 0.0622 | 0.0374 | 0.0054 |
| CAP45.G3 | C | 5.7760 | 0.1119 | 0.0734 | 0.0021 |
| 3016.v5.c45 | D | >50 | 0.5160 | 0.6111 | 0.0156 |
| 231965.c01 | D | >50 | 0.1963 | 0.2164 | 0.0157 |
| X1632.S2.B10 | G | >50 | 0.2703 | 0.9352 | 0.0600 |

The synergistic 13-fold increase of the BiS13 bispecific antibody's neutralization potency compared to that of the parental antibodies against the panel of pseudoviruses shown in Table 4 was surprising and unexpected. While multiple reports on designing HIV-1 bi- and trispecific antibodies showed that the bi- and trispecific antibodies have increased breadth of neutralization compared to parental monoclonal antibodies, the gain of breadth in most cases does not correlate with improved potency. See, e.g., Padte, N. N., Yu, J., Huang, Y. et al. Engineering multi-specific antibodies against HIV-1. Retrovirology 15, 60 (2018), DOI: 10.1186/s12977-018-0439-9; Ling Xu et al., Trispecific broadly neutralizing HIV antibodies mediate potent SHIV protection in macaques. Science 358,85-90(2017), DOI: 10.1126/science.aan8630. Improved neutralization potency was reported for a trispecific antibody combining scFv domains connected via flexible linkers [Steinhardt, J. J., Guenaga, J., Turner, H. L. et al. Rational design of a trispecific antibody targeting the HIV-1 Env with elevated anti-viral activity. Nat Commun 9, 877 (2018), DOI: 10.1038/s41467-018-03335-4] and for a bispecific antibody utilizing a long and flexible IgG3 hinge-like domain [Bournazos S., Gazumyan A., Seaman M. S., Nussenzweig M. C., Ravetch J. V. Bispecific Anti-HIV-1 Antibodies with Enhanced Breadth and Potency. Cell 165(7):1609-1620 (2016), DOI: 10.1016/j.cell.2016.04.050]. There are no reports of a bispecific antibody using the general architecture of BiS13 achieving the synergistic increase in neutralization potency observed with BiS13.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

SEQUENCES

| | |
|---|---|
| SEQ ID NO: 1 | ePGT121v1 VH CDR1 |
| SEQ ID NO: 2 | ePGT121v1 VH CDR2 |
| SEQ ID NO: 3 | ePGI121v1 VH CDR3 |
| SEQ ID NO: 4 | ePGT121v1 VH |
| SEQ ID NO: 5 | ePGT121v1 VL CDR1 |
| SEQ ID NO: 6 | ePGT121v1 VL CDR2 |
| SEQ ID NO: 7 | ePGT121v1 VL CDR3 |
| SEQ ID NO: 8 | ePGT121v1 VL |
| SEQ ID NO: 9 | ePGT121v1 scFV/12 |
| SEQ ID NO: 10 | ePGI121v1 scFV/12 B |
| SEQ ID NO: 11 | ePGT121v1 scFV/14 |
| SEQ ID NO: 12 | ePGT121v1 scFV/14 B |
| SEQ ID NO: 13 | ePGT121v1 scFv/12-Fc knob (Bis 1, 3, 9 |
| | 1; Tris 5 & 6 Chain 1) |
| SEQ ID NO: 14 | ePGT121v1 scFv/14-Fc knob (Bis 2, 4, 10 |
| | 1) |
| SEQ ID NO: 15 | ePGT121v1 HC |
| SEQ ID NO: 16 | ePGT121v1 HC hole (Tris 1 & 2 Chain 2) |
| SEQ ID NO: 17 | ePGT121v1 Fab heavy |
| SEQ ID NO: 18 | ePGT121v1 LC |
| SEQ ID NO: 19 | ePGT121v2 VH CDR1 |
| SEQ ID NO: 20 | ePGT121v2 VH CDR2 |
| SEQ ID NO: 21 | ePGI121v2 VH CDR3 |
| SEQ ID NO: 22 | ePGT121v2 VH |
| SEQ ID NO: 23 | ePGT121v2 VL CDR1 |
| SEQ ID NO: 24 | ePGT121v2 VL CDR2 |
| SEQ ID NO: 25 | ePGT121v2 VL CDR3 |
| SEQ ID NO: 26 | ePGT121v2 VL |
| SEQ ID NO: 27 | ePGT121v2 HC (BiS 13 & 14 Chain 1) |
| SEQ ID NO: 28 | ePGT121v2 Fab heavy |
| SEQ ID NO: 29 | ePGT121v2 LC |
| SEQ ID NO: 30 | ePGDM1400v9 VH CDR1 |
| SEQ ID NO: 31 | ePGDM1400v9 VH CDR2 |
| SEQ ID NO: 32 | ePGDM1400v9 VH CDR3 |
| SEQ ID NO: 33 | ePGDM1400v9 VH |
| SEQ ID NO: 34 | ePGDM1400v9 VL CDR1 |
| SEQ ID NO: 35 | ePGDM1400v9 VL CDR2 |
| | LAS |
| SEQ ID NO: 36 | ePGDM1400v9 VL CDR3 |
| SEQ ID NO: 37 | ePGDM1400v9 Fab heavy |
| SEQ ID NO: 38 | ePGDM1400v9 VL |
| SEQ ID NO: 39 | ePGDM1400v9 VH-VL scFv |
| SEQ ID NO: 40 | ePGDM1400v9 VL-VH scFv |
| SEQ ID NO: 41 | ePGDM1 400v9 VH-VL scFv-Fc knob (Bis 5 & 7 Chain |
| | 1; TriS 1 Chain 1) |
| SEQ ID NO: 42 | ePGDM1400v9 VL-VH scFv-Fc knob (Bis 6 & 8 Chain |
| | 1; TriS 2 Chain 1) |
| SEQ ID NO: 43 | ePGDM1 400v9 VH-VL scFv-Fc hole (Bis 11 & 12 Chain |
| | 2) |
| SEQ ID NO: 44 | ePGDM1400v9 VL-VH scFv-Fc hole (Bis 9 & 10 Chain |
| | 2) |
| SEQ ID NO: 45 | N49P7 VH CDR1 |
| SEQ ID NO: 46 | N49P7 VH CDR2 |
| SEQ ID NO: 47 | N49P7 VH CDR3 |
| SEQ ID NO: 48 | N49P7 VH |
| SEQ ID NO: 49 | N49P7 FR3 VH |
| SEQ ID NO: 50 | N49P7 VL CDR1 HNL |
| SEQ ID NO: 51 | N49P7 VL CDR2 DEN |
| SEQ ID NO: 52 | N49P7 VL CDR3 |
| SEQ ID NO: 53 | N49P7 VL |
| SEQ ID NO: 54 | N49P7 scFv |
| SEQ ID NO: 55 | N49P7 FR3 scFv |
| SEQ ID NO: 56 | N49P7 HC hole (Bis 1, 2, 5 & 6 Chain 2) |
| SEQ ID NO: 57 | N49P7 FR3 HC hole (Bis 3, 4, 7& 8 Chain 2; Tris 3 |
| | & 4 Chain 2) |
| SEQ ID NO: 58 | N49P7 Fab heavy |
| SEQ ID NO: 59 | N49P7 FR3 Fab heavy |
| SEQ ID NO: 60 | N49P7 LC (Bis 1, 2, 3, 4, 5, 6, 7 & 8 Chain 3; |
| | TriS 3, 4, 5 & 6 Chain 3) |
| SEQ ID NO: 61 | N49P7 scFv-24aa-ePGT121v2 LC (BiS 13 Chain 2) |
| SEQ ID NO: 62 | N49P7 FR3 scFv-24aa-ePGT121v2 LC (Bis 14 Chain 2) |
| SEQ ID NO: 63 | ePGT121v2 HC hole |
| SEQ ID NO: 64 | N49P7 FR3 scFv-ePGT121v1 LC |
| SEQ ID NO: 65 | ePGT121v1 scFv/12-Fc knob-ePGDM1400v9 VH/VL scFv |
| | (TriS 3 Chain 1) |
| SEQ ID NO: 66 | ePGT121v1 scFv/12-Fc knob-ePGDM1400v9 VL/VH scFv |
| | (Tris 4 Chain 1) |
| SEQ ID NO: 67 | N49P7-FR3 HC hole-ePGDM1400v9 VH/VL scFv (Tris |
| | 5 Chain 2) |
| SEQ ID NO: 68 | N49P7-FR3 HC hole-ePGDM1400v9 VL/VH scFv (TriS |
| | 6 Chain 2) |
| SEQ ID NO: 69 | signal peptide A |
| SEQ ID NO: 70 | signal peptide B |
| SEQ ID NO: 71 | Linker |
| SEQ ID NO: 72 | Linker |
| SEQ ID NO: 73 | Linker |
| SEQ ID NO: 74 | Linker |
| SEQ ID NO: 75 | Linker |
| SEQ ID NO: 76 | Fc |
| SEQ ID NO: 77 | Fc knob |
| SEQ ID NO: 78 | Fc hole |
| SEQ ID NO: 79 | CH1/CH2/CH3 |
| SEQ ID NO: 80 | CH1/CH2/CH3 knob |
| SEQ ID NO: 81 | CH1/CH2/CH3 hole |
| SEQ ID NO: 82 | BiS1 chain 1 |
| SEQ ID NO: 83 | Bis1 chain 2 |
| SEQ ID NO: 84 | Bis1 chain 3 |
| SEQ ID NO: 85 | Bis2 chain 1 |
| SEQ ID NO: 86 | Bis2 chain 2 |
| SEQ ID NO: 87 | Bis2 chain 3 |
| SEQ ID NO: 88 | Bis3 chain 1 |
| SEQ ID NO: 89 | Bis3 chain 2 |
| SEQ ID NO: 90 | BiS3 chain 3 |
| SEQ ID NO: 91 | Bis4 chain 1 |
| SEQ ID NO: 92 | Bis4 chain 2 |
| SEQ ID NO: 93 | Bis4 chain 3 |
| SEQ ID NO: 94 | Bis5 chain 1 |
| SEQ ID NO: 95 | Bis5 chain 2 |
| SEQ ID NO: 96 | Bis5 chain 3 |
| SEQ ID NO: 97 | BiS6 chain 1 |
| SEQ ID NO: 98 | BiS6 chain 2 |
| SEQ ID NO: 99 | BiS6 chain 3 |
| SEQ ID NO: 100 | Bis7 chain 1 |
| SEQ ID NO: 101 | Bis7 chain 2 |
| SEQ ID NO: 102 | BiS7 chain 3 |
| SEQ ID NO: 103 | BiS8 chain 1 |
| SEQ ID NO: 104 | Bis8 chain 2 |
| SEQ ID NO: 105 | Bis8 chain 3 |
| SEQ ID NO: 106 | Bis9 chain 1 |
| SEQ ID NO: 107 | Bis9 chain 2 |
| SEQ ID NO: 108 | BiS10 chain 1 |
| SEQ ID NO: 109 | BiS10 chain 2 |
| SEQ ID NO: 110 | BiS11 chain 1 |
| SEQ ID NO: 111 | Bis11 chain 2 |
| SEQ ID NO: 112 | Bis12 chain 1 |
| SEQ ID NO: 113 | Bis12 chain 2 |
| SEQ ID NO: 114 | Bis13 chain 1 |
| SEQ ID NO: 115 | Bis13 chain 2 |
| SEQ ID NO: 116 | Bis14 chain 1 |
| SEQ ID NO: 117 | Bis14 chain 2 |
| SEQ ID NO: 118 | TriS1 chain 1 |
| SEQ ID NO: 119 | Tris1 chain 2 |
| SEQ ID NO: 120 | TriS1 chain 3 |
| SEQ ID NO: 121 | Tris2 chain 1 |
| SEQ ID NO: 122 | TriS2 chain 2 |
| SEQ ID NO: 123 | Tris2 chain 3 |
| SEQ ID NO: 124 | TriS3 chain 1 |
| SEQ ID NO: 125 | TriS3 chain 2 |
| SEQ ID NO: 126 | TriS3 chain 3 |
| SEQ ID NO: 127 | Tris4 chain 1 |
| SEQ ID NO: 128 | Tris4 chain 2 |
| SEQ ID NO: 129 | Tris4 chain 3 |
| SEQ ID NO: 130 | Tris5 chain 1 |
| SEQ ID NO: 131 | TriS5 chain 2 |
| SEQ ID NO: 132 | TriS5 chain 3 |
| SEQ ID NO: 133 | TriS6 chain 1 |
| SEQ ID NO: 134 | TriS6 chain 2 |
| SEQ ID NO: 135 | TriS6 chain 3 |
| SEQ ID NO: 136 | signal peptide |
| SEQ ID NO: 137 | signal peptide B |
| SEQ ID NO: 138 | CL |
| SEQ ID NO: 139 | Linker |
| SEQ ID NO: 140 | Linker |
| SEQ ID NO: 141 | Linker |
| SEQ ID NO: 142 | N49P7 FR3 scFv-34aa-ePGT121v2 LC (Tris1 & 2 |
| | Chain3) |
| SEQ ID NO: 143 | Linker |

What is claimed is:

1. A multispecific anti-HIV antibody comprising at least two antigen binding domains selected from the group consisting of
(a) an antigen binding domain that binds to the V3 loop epitope region of HIV Env, wherein the antigen binding domain comprises a VH domain and a VL domain comprising the 3 VH CDRs and 3 VL CDRs, respectively, of ePGT121v2 or ePGT121v1; and
(b) an antigen binding domain that binds to the CD4bs epitope region of HIV Env, wherein the antigen binding domain comprises a VH domain and a VL domain comprising the 3 VH CDRs and 3 VL CDRs, respectively, of N49P7 or an engineered variant of N49P7.

2. The multispecific antibody of claim 3, wherein the ePGT121v2 VH CDR1, VH CDR2, VH CDR3, VL CDR1, VL CDR2 and VL CDR3 comprise the amino acid sequence of SEQ ID NO: 19, 20, 21, 23, 24 and 25, respectively, and the ePGT121v1 VH CDR1, VH CDR2, VH CDR3, VL CDR1, VL CDR2 and VL CDR3 of ePGT121v1 comprise the amino acid sequence of SEQ ID NO: 1, 2, 3, 5, 6, and 7, respectively.

3. The multispecific antibody of claim 1 or 2, wherein the ePGT121v2 VH domain and VL domain comprises an amino acid sequence having at least 80%, at least 85%, at least 90%, at least 95%, at least 97% or 100% identity to SEQ ID NO: 22 and 26, respectively, and the ePGT121v1 VH domain and VL domain comprises an amino acid sequence having at least 80%, at least 85%, at least 90%, at least 95%, at least 97% or 100% identity to SEQ ID NO: 4 and 8, respectively.

4. The multispecific antibody of claim 3, wherein the ePGT121v2 antigen binding domain comprises an Fab domain comprising a heavy chain and light chain having the amino acid sequence of SEQ ID NO: 28 and 29, and the ePGT121v1 antigen binding domain comprises an Fab domain comprising a heavy chain and light chain having the amino acid sequence of SEQ ID NO: 17 and 18, respectively.

5. The multispecific antibody of any one of claims 1 to 4, wherein the VH domain comprises the VH CDR1, VH CDR2, VH CDR3 of N49P7, and the VL domain comprises the VL CDR1, VL CDR2 and VL CDR3 of N49P7.

6. The multispecific antibody of claim 5, wherein the N49P7 VH CDR1, VH CDR2, VH CDR3, VL CDR1, VL CDR2 and VL CDR3 comprise the amino acid sequence of SEQ ID NO: 45, 46, 47, 50, 51 and 52, respectively.

7. The multispecific antibody of any one of claims 1 to 6, wherein the N49P7 VH domain and VL domain comprises an amino acid sequence having at least 80%, at least 85%, at least 90%, at least 95%, at least 97% or 100% identity to SEQ ID NO: 48 and 53, respectively, or SEQ ID NO: 49 and 53, respectively.

8. The multispecific antibody of any one of claims 1 to 7, wherein the antigen binding domain that binds to the CD4bs epitope region of HIV Env comprises an scFv domain.

9. The multispecific antibody of claim 8, wherein the antigen binding domain comprises an scFv comprising the amino acid sequence of SEQ ID NO: 54 or 55.

10. The multispecific antibody of any one of claims 1 to 9, which is a bispecific antibody.

11. The multispecific antibody of claim 10 comprising an scFv domain that binds to the CD4bs epitope region of HIV Env linked to an antibody that binds to the V3 loop epitope region of HIV Env.

12. The multispecific antibody of claim 11, wherein the C-terminal end of the scFv domain is linked to the N-terminal end of the antibody light chain by a polypeptide linker, wherein the linker comprises between 15 and 35 amino acid residues.

13. The multispecific antibody of claim 12, wherein the linker comprises between 20 and 30 amino acid residues, between 20 and 27 amino acid residues, between 23 and 30 amino acid residues, or between 23 and 27 amino acid residues.

14. The multispecific antibody of claim 12, wherein the linker comprises 20, 21, 22, 23, 24, 25, 26, 27, 28, 29 or 30 amino acid residues, optionally wherein the linker comprises 23, 24, 25, 26, or 27 amino acid residues.

15. The multispecific antibody of any one of claims 12 to 14, wherein the linker comprises one or more copies of the amino acid sequence of SEQ ID NO: 141.

16. The multispecific antibody of any one of claims 12 to 14, wherein the linker comprises the amino acid sequence of SEQ ID NO: 140.

17. The multispecific antibody of claim 11, wherein the C-terminal end of the scFv domain is linked to the N-terminal end of the antibody light chain by a polypeptide linker, wherein the linker comprises between 25 and 45 amino acid residues.

18. The multispecific antibody of claim 17, wherein the linker comprises between 30 and 40 amino acid residues, between 30 and 37 amino acid residues, between 33 and 40 amino acid residues, or between 33 and 37 amino acid residues.

19. The multispecific antibody of claim 17, wherein the linker comprises 30, 31, 32, 33, 34, 35, 36, 37, 38, 39 or 40 amino acid residues, optionally wherein the linker comprises 33, 34, 35, 36, or 37 amino acid residues.

20. The multispecific antibody of any one of claims 17 to 19, wherein the linker comprises one or more copies of the amino acid sequence of SEQ ID NO: 141.

21. The multispecific antibody of any one of claims 17 to 19, wherein the linker comprises the amino acid sequence of SEQ ID NO: 143.

22. The multispecific antibody of any one of claims 11 to 21, wherein the scFv linked to the antibody light chain and the antibody heavy chain comprises an amino acid sequence having at least 80%, at least 85%, at least 90%, at least 95%, at least 97% or 100% identity to SEQ ID NO: 62 and 27, respectively, to SEQ ID NO: 61 and 27, respectively, or to SEQ ID NO: 142 and 27, respectively.

23. An isolated polynucleotide encoding the multispecific antibody of any one of claims 1 to 22.

24. The isolated polynucleotide of claim 23, wherein the polynucleotide comprises a nucleotide sequence encoding a signal peptide.

25. The isolated polynucleotide of claim 24, wherein the signal peptide comprises the amino acid sequence of SEQ ID NO: 69 or 70.

26. The polynucleotide of any one of claims 23 to 25 that is DNA.

27. The polynucleotide of any one of claims 23 to 25 that is RNA.

28. The polynucleotide of claim 27, wherein the RNA is mRNA comprising a modified ribonucleotide.

29. A vector comprising the polynucleotide of any one of claims 23 to 25.

30. The vector of claim 29 comprising more than one isolated polynucleotide.

31. A host cell capable of producing the multispecific antibody of any one of claims 1 to 22.

32. A host cell comprising the polynucleotide of any one of claims 23 to 25 or the vector of claim 29 or 30.

33. The host cell of claim 32 which is a CHO cell or a HEK293 cell.

34. A recombinant virus comprising the polynucleotide of any one of claims 23 to 25.

35. A pharmaceutical composition comprising the multispecific antibody of any one of claims 1 to 22 and a pharmaceutically acceptable excipient.

36. A pharmaceutical composition comprising the isolated polynucleotide of any one of claims 23 to 28 and a pharmaceutically acceptable excipient.

37. The pharmaceutical composition of claim 36, wherein the polynucleotide is RNA.

38. The pharmaceutical composition of claim 37, wherein the RNA is mRNA comprising a modified ribonucleotide.

39. A method of neutralizing an HIV virus comprising contacting the virus with a sufficient amount of a multispecific antibody of any one of claims 1 to 22 or a pharmaceutical composition according to any one of claims 35 to 38.

40. A method of reducing the likelihood of HIV infection in a subject exposed to HIV comprising administering to the subject a therapeutically sufficient amount of a multispecific antibody of any one of claims 1 to 22 or a pharmaceutical composition according to any one of claims 35 to 38.

41. A method of treating HIV/AIDS comprising administering to a subject in need thereof a therapeutically sufficient amount of a multispecific antibody of any one of claims 1 to 22 or a pharmaceutical composition according to any one of claims 35 to 38.

42. A method of reducing viral load comprising administering to a subject in need thereof a therapeutically sufficient amount of a multispecific antibody of any one of claims 1 to 22 or a pharmaceutical composition according to any one of claims 35 to 38.

43. The method of any one of claims 40 to 42 further comprising administering at least one additional therapeutic agent.

44. The method of claim 43, wherein the additional therapeutic agent is an antiretroviral agent, a reservoir activator, or an additional antibody.

45. The method of claim 44, wherein the additional therapeutic agent comprises an additional antibody, which is broadly neutralizing antibody.


SEQUENCE LISTING (143 sequences)

SEQ ID NO: 1 - protein, 5 aa, synthetic construct
SSYWS

SEQ ID NO: 2 - protein, 16 aa, synthetic construct
YTHHSGDTNYAPSLKS

SEQ ID NO: 3 - protein, 24 aa, synthetic construct
TLHGRRIYGVVAFNEFFTYFYWEV

SEQ ID NO: 4 - protein, 132 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSS

SEQ ID NO: 5 - protein, 11 aa, synthetic construct
GGESIGSRAVQ

SEQ ID NO: 6 - protein, 7 aa, synthetic construct
NNQDRPP

SEQ ID NO: 7 - protein, 12 aa, synthetic construct
HIWDSRRPTNWV

SEQ ID NO: 8 - protein, 105 aa, synthetic construct
SDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVL

SEQ ID NO: 9 - protein, 249 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSSGGVGGEGGGGSGSDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVL

SEQ ID NO: 10 - protein, 251 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSSGGVGGEGGGGSGSDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVLGQ

SEQ ID NO: 11 - protein, 251 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSSGSVGSVSGSGGGGASDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVL

SEQ ID NO: 12 - protein, 253 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSSGSVGSVSGSGGGGASDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVLGQ

SEQ ID NO: 13 - protein, 507 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSSGGVGGEGGGGSGSDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVLGQGGSGGSGGSGGSGGSGGSGGSGGSGGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 509 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSSGSVGSVSGSGGGGASDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVLGQGGSGGSGGSGGSGGSGGSGGSGGSGGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 462 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEYYTYFYWEVWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 16 - protein, 462 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 17 - protein, 235 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEYYTYFYWEVWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 211 aa, synthetic construct
SDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 19 - protein, 5 aa, synthetic construct
SSYWS

SEQ ID NO: 20 - protein, 16 aa, synthetic construct
YTHHSGDTNYAPSLKS

SEQ ID NO: 21 - protein, 24 aa, synthetic construct
TLHGRRIYGVVAFNEYYTYFYWPT

SEQ ID NO: 22 - protein, 132 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEYYTYFYWPTWGKGTQVTVSS

SEQ ID NO: 23 - protein, 11 aa, synthetic construct
GGESIGSRAVQ

SEQ ID NO: 24 - protein, 7 aa, synthetic construct
NNQDRPP

SEQ ID NO: 25 - protein, 12 aa, synthetic construct
HIWDSRRPTNWE

SEQ ID NO: 26 - protein, 105 aa, synthetic construct
SDISVAPGETVRITCGGESIGSRAVQWYQHRPGQAPRLIIYNNQDRPPGIPERFSGSPDIDFGTTATLTISNVEAGDEATYYCHIWDSRRPTNWELGPGTTLTVL

SEQ ID NO: 27 - protein, 462 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEYYTYFYWPTWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 28 - protein, 235 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEYYTYFYWPTWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 29 - protein, 211 aa, synthetic construct
SDISVAPGETVRITCGGESIGSRAVQWYQHRPGQAPRLIIYNNQDRPPGIPERFSGSPDIDFGTTATLTISNVEAGDEATYYCHIWDSRRPTNWELGPGTTLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 30 - protein, 8 aa, synthetic construct
GNTLTTYD

SEQ ID NO: 31 - protein, 8 aa, synthetic construct
ISHEGDKK

SEQ ID NO: 32 - protein, 36 aa, synthetic construct
CAKGSKHRLRDYALYDEDGALNWAVDVDYLANLEFW

SEQ ID NO: 33 - protein, 141 aa, synthetic construct
QAQLVQSGPEVRKPGTSVKVSCKNPGNTLTTYDLHWVRQVPGQGLQWMGWISHEGDKKVIVERFKAKVTIDWDRSTNTAYLQLSGLTSEDTAVYYCAKGSKHRLRDYALYDEDGALNWAVDVDYLANLEFWGQGTAVTVSS

SEQ ID NO: 34 - protein, 11 aa, synthetic construct
QSLIHGDRNNY

SEQ ID NO: 35 - skipped

SEQ ID NO: 36 - protein, 11 aa, synthetic construct
CMQGRESPWTF

SEQ ID NO: 37 - protein, 244 aa, synthetic construct
QAQLVQSGPEVRKPGTSVKVSCKNPGNTLTTYDLHWVRQVPGQGLQWMGWISHEGDKKVIVERFKAKVTIDWDRSTNTAYLQLSGLTSEDTAVYYCAKGSKHRLRDYALYDEDGALNWAVDVDYLANLEFWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 38 - protein, 112 aa, synthetic construct
DIVLTQSPLSLSVTPGESASISCKSSQSLIHGDRNNYLAWYVQKPGRSPQLLIDLASERASGVPDRFSGSGSRKDFTLKISRVETEDVGTYYCMQGRESPWTFGQGTKVDIK

SEQ ID NO: 39 - protein, 268 aa, synthetic construct
QAQLVQSGPEVRKPGTSVKVSCKNPGNTLTTYDLHWVRQVPGQGLQWMGWISHEGDKKVIVERFKAKVTIDWDRSTNTAYLQLSGLTSEDTAVYYCAKGSKHRLRDYALYDEDGALNWAVDVDYLANLEFWGQGTAVTVSSGGGGSGGGGSGGGGSDIVLTQSPLSLSVTPGESASISCKSSQSLIHGDRNNYLAWYVQKPGRSPQLLIDLASERASGVPDRFSGSGSRKDFTLKISRVETEDVGTYYCMQGRESPWTFGQGTKVDIK

SEQ ID NO: 40 - protein, 268 aa, synthetic construct
DIVLTQSPLSLSVTPGESASISCKSSQSLIHGDRNNYLAWYVQKPGRSPQLLIDLASERASGVPDRFSGSGSRKDFTLKISRVETEDVGTYYCMQGRESPWTFGQGTKVDIKGGGGSGGGGSGGGGSQAQLVQSGPEVRKPGTSVKVSCKNPGNTLTTYDLHWVRQVPGQGLQWMGWISHEGDKKVIVERFKAKVTIDWDRSTNTAYLQLSGLTSEDTAVYYCAKGSKHRLRDYALYDEDGALNWAVDVDYLANLEFWGQGTAVTVSS

SEQ ID NO: 41 - protein, 524 aa, synthetic construct
QAQLVQSGPEVRKPGTSVKVSCKNPGNTLTTYDLHWVRQVPGQGLQWMGWISHEGDKKVIVERFKAKVTIDWDRSTNTAYLQLSGLTSEDTAVYYCAKGSKHRLRDYALYDEDGALNWAVDVDYLANLEFWGQGTAVTVSSGGGGSGGGGSGGGGSDIVLTQSPLSLSVTPGESASISCKSSQSLIHGDRNNYLAWYVQKPGRSPQLLIDLASERASGVPDRFSGSGSRKDFTLKISRVETEDVGTYYCMQGRESPWTFGQGTKVDIKGGSGGSGGSGGSGGSGGSGGSGGSGGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 524 aa, synthetic construct
DIVLTQSPLSLSVTPGESASISCKSSQSLIHGDRNNYLAWYVQKPGRSPQLLIDLASERASGVPDRFSGSGSRKDFTLKISRVETEDVGTYYCMQGRESPWTFGQGTKVDIKGGGGSGGGGSGGGGSQAQLVQSGPEVRKPGTSVKVSCKNPGNTLTTYDLHWVRQVPGQGLQWMGWISHEGDKKVIVERFKAKVTIDWDRSTNTAYLQLSGLTSEDTAVYYCAKGSKHRLRDYALYDEDGALNWAVDVDYLANLEFWGQGTAVTVSSGGSGGSGGSGGSGGSGGSGGSGGSGGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 524 aa, synthetic construct
QAQLVQSGPEVRKPGTSVKVSCKNPGNTLTTYDLHWVRQVPGQGLQWMGWISHEGDKKVIVERFKAKVTIDWDRSTNTAYLQLSGLTSEDTAVYYCAKGSKHRLRDYALYDEDGALNWAVDVDYLANLEFWGQGTAVTVSSGGGGSGGGGSGGGGSDIVLTQSPLSLSVTPGESASISCKSSQSLIHGDRNNYLAWYVQKPGRSPQLLIDLASERASGVPDRFSGSGSRKDFTLKISRVETEDVGTYYCMQGRESPWTFGQGTKVDIKGGSGGSGGSGGSGGSGGSGGSGGSGGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - protein, 524 aa, synthetic construct
DIVLTQSPLSLSVTPGESASISCKSSQSLIHGDRNNYLAWYVQKPGRSPQLLIDLASERASGVPDRFSGSGSRKDFTLKISRVETEDVGTYYCMQGRESPWTFGQGTKVDIKGGGGSGGGGSGGGGSQAQLVQSGPEVRKPGTSVKVSCKNPGNTLTTYDLHWVRQVPGQGLQWMGWISHEGDKKVIVERFKAKVTIDWDRSTNTAYLQLSGLTSEDTAVYYCAKGSKHRLRDYALYDEDGALNWAVDVDYLANLEFWGQGTAVTVSSGGSGGSGGSGGSGGSGGSGGSGGSGGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 45 - protein, 8 aa, synthetic construct
GYRFPDYI

SEQ ID NO: 46 - protein, 8 aa, synthetic construct
MNPMGGQV

SEQ ID NO: 47 - protein, 21 aa, synthetic construct
VRDRSNGSGKRFESSNWFLDL

SEQ ID NO: 48 - protein, 127 aa, synthetic construct
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQS

SEQ ID NO: 49 - protein, 134 aa, synthetic construct
ADLVQSGAVVKKPGDSVRISCEAQGYRFPDYIIHWIRRAPGQGPEWMGWMNPMGGQVNIPWKFQGRVSMTRQLSQDPDDPDWGTAFLDLRGLKSDDTAVYYCVRDRSNGSGKRFESSNWFLDLWGRGTAVTIQS

SEQ ID NO: 50-51 - skipped (2 entries)

SEQ ID NO: 52 - protein, 5 aa, synthetic construct
WAYEA

SEQ ID NO: 53 - protein, 97 aa, synthetic construct
ALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVL

SEQ ID NO: 54 - protein, 241 aa, synthetic construct
ALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQGGGGSGGGGSGGGGSADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQS

SEQ ID NO: 55 - protein, 248 aa, synthetic construct
ALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQGGGGSGGGGSGGGGSADLVQSGAVVKKPGDSVRISCEAQGYRFPDYIIHWIRRAPGQGPEWMGWMNPMGGQVNIPWKFQGRVSMTRQLSQDPDDPDWGTAFLDLRGLKSDDTAVYYCVRDRSNGSGKRFESSNWFLDLWGRGTAVTIQS

SEQ ID NO: 56 - protein, 457 aa, synthetic construct
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 57 - protein, 464 aa, synthetic construct
ADLVQSGAVVKKPGDSVRISCEAQGYRFPDYIIHWIRRAPGQGPEWMGWMNPMGGQVNIPWKFQGRVSMTRQLSQDPDDPDWGTAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 58 - protein, 230 aa, synthetic construct
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 59 - protein, 237 aa, synthetic construct
ADLVQSGAVVKKPGDSVRISCEAQGYRFPDYIIHWIRRAPGQGPEWMGWMNPMGGQVNIPWKFQGRVSMTRQLSQDPDDPDWGTAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 60 - protein, 203 aa, synthetic construct
ALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECG

SEQ ID NO: 61 - protein, 476 aa, synthetic construct
ALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQGGGGSGGGGSGGGGSADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSGGGGSGGGGSGGGGSGGGGSGGGGSDISVAPGETVRITCGGESIGSRAVQWYQHRPGQAPRLIIYNNQDRPPGIPERFSGSPDIDFGTTATLTISNVEAGDEATYYCHIWDSRRPTNWELGPGTTLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 62 - protein, 483 aa, synthetic construct
ALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQGGGGSGGGGSGGGGSADLVQSGAVVKKPGDSVRISCEAQGYRFPDYIIHWIRRAPGQGPEWMGWMNPMGGQVNIPWKFQGRVSMTRQLSQDPDDPDWGTAFLDLRGLKSDDTAVYYCVRDRSNGSGKRFESSNWFLDLWGRGTAVTIQSGGGGSGGGGSGGGGSGGGGSGGGGSDISVAPGETVRITCGGESIGSRAVQWYQHRPGQAPRLIIYNNQDRPPGIPERFSGSPDIDFGTTATLTISNVEAGDEATYYCHIWDSRRPTNWELGPGTTLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 63 - protein, 462 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWPTWGKGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 64 - protein, 483 aa, synthetic construct
ALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQGGGGSGGGGSGGGGSADLVQSGAVVKKPGDSVRISCEAQGYRFPDYIIHWIRRAPGQGPEWMGWMNPMGGQVNIPWKFQGRVSMTRQLSQDPDDPDWGTAFLDLRGLKSDDTAVYYCVRDRSNGSGKRFESSNWFLDLWGRGTAVTIQSGGGGSGGGGSGGGGSGGGGSGGGGSDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 65 - protein, 790 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSSGGVGGEGGGGSGSDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVLGQGGSGGSGGSGGSGGSGGSGGSGGSGGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQAQLVQSGPEVRKPGTSVKVSCKNPGNTLTTYDLHWVRQVPGQGLQWMGWISHEGDKKVIVERFKAKVTIDWDRSTNTAYLQLSGLTSEDTAVYYCAKGSKHRLRDYALYDEDGALNWAVDVDYLANLEFWGQGTAVTVSSGGGGSGGGGSGGGGSDIVLTQSPLSLSVTPGESASISCKSSQSLIHGDRNNYLAWYVQKPGRSPQLLIDLASERASGVPDRFSGSGSRKDFTLKISRVETEDVGTYYCMQGRESPWTFGQGTKVDIK

SEQ ID NO: 66 - protein, 790 aa, synthetic construct
QMQLQESGPGLVKPGETLSLTCSVSGASISSSYWSWLRETPGKGLEWIGYTHHSGDTNYAPSLKSRVHLGLHPSKNQVSLSLTSVTAADTAVYYCARTLHGRRIYGVVAFNEFFTYFYWEVWGKGTQVTVSSGGVGGEGGGGSGSDISVAPGETVRISCGGESIGSRAVQWYQHRAGQAPKLIIYNNQDRPPGIPERFSGSPDIDFGTTATLTITNVEAGDEATYYCHIWDSRRPTNWVFGGGTTLTVLGQGGSGGSGGSGGSGGSGGSGGSGGSGGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIVLTQSPLSLSVTPGESASISCKSSQSLIHGDRNNYLAWYVQKPGRSPQLLIDLASERASGVPDRFSGSGSRKDFTLKISRVETEDVGTYYCMQGRESPWTFGQGTKVDIKGGGGSGGGGSGGGGSQAQLVQSGPEVRKPGTSVKVSCKNPGNTLTTYDLHWVRQVPGQGLQWMGWISHEGDKKVIVERFKAKVTIDWDRSTNTAYLQLSGLTSEDTAVYYCAKGSKHRLRDYALYDEDGALNWAVDVDYLANLEFWGQGTAVTVSS

SEQ ID NO: 67 - protein, 747 aa, synthetic construct
ADLVQSGAVVKKPGDSVRISCEAQGYRFPDYIIHWIRRAPGQGPEWMGWMNPMGGQVNIPWKFQGRVSMTRQLSQDPDDPDWGTAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQAQLVQSGPEVRKPGTSVKVSCKNPGNTLTTYDLHWVRQVPGQGLQWMGWISHEGDKKVIVERFKAKVTIDWDRSTNTAYLQLSGLTSEDTAVYYCAKGSKHRLRDYALYDEDGALNWAVDVDYLANLEFWGQGTAVTVSSGGGGSGGGGSGGGGSDIVLTQSPLSLSVTPGESASISCKSSQSLIHGDRNNYLAWYVQKPGRSPQLLIDLASERASGVPDRFSGSGSRKDFTLKISRVETEDVGTYYCMQGRESPWTFGQGTKVDIK

SEQ ID NO: 68 - protein, 747 aa, synthetic construct
ADLVQSGAVVKKPGDSVRISCEAQGYRFPDYIIHWIRRAPGQGPEWMGWMNPMGGQVNIPWKFQGRVSMTRQLSQDPDDPDWGTAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSDIVLTQSPLSLSVTPGESASISCKSSQSLIHGDRNNYLAWYVQKPGRSPQLLIDLASERASGVPDRFSGSGSRKDFTLKISRVETEDVGTYYCMQGRESPWTFGQGTKVDIKGGGGSGGGGSGGGGSQAQLVQSGPEVRKPGTSVKVSCKNPGNTLTTYDLHWVRQVPGQGLQWMGWISHEGDKKVIVERFKAKVTIDWDRSTNTAYLQLSGLTSEDTAVYYCAKGSKHRLRDYALYDEDGALNWAVDVDYLANLEFWGQGTAVTVSS

SEQ ID NO: 69 - protein, 19 aa, synthetic construct
MDWTWRILFLVAAATGAHS

SEQ ID NO: 70 - protein, 20 aa, synthetic construct
METDTLLLWVLLLWVPGSTG

SEQ ID NO: 71 - protein, 12 aa, synthetic construct
GGVGGEGGGGSG

SEQ ID NO: 72 - protein, 14 aa, synthetic construct
GSVGSVSGSGGGGA

SEQ ID NO: 73 - protein, 14 aa, synthetic construct
GSVGSVSGSGGGGA

SEQ ID NO: 74 - protein, 15 aa, synthetic construct
GGGGSGGGGSGGGGS

SEQ ID NO: 75 - protein, 29 aa, synthetic construct
GGSGGSGGSGGSGGSGGSGGSGGSGGSGG

SEQ ID NO: 76 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 77 - protein, 227 aa, synthetic construct [sequence omitted]

SEQ ID NO: 78 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 79 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 80 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVLHEALHNHYTQKSLSLSPGK

SEQ ID NO: 81 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 82 - DNA, 1521 bp, synthetic construct
  source 1..1521: mol_type = other DNA
caaatgcagctgcaggagagcggtcctggcctggtgaagcctggcgagacactgagcctgacatgcagcgtgtccggcgccagcatcagcagcagctactggagctggctgcgggaaacccctggaaaaggcctggaatggatcggctacacccaccacagcggcgacacaaattacgccccaagcctcaagtccagagtgcacctgggcctgcaccccagcaagaaccaggtgtccctgtctctgaccagcgtcaccgcagccgacaccgctgtgtactactgcgccagaaccctgcatggcagaaggatctacggcgttgtggcctttaacgagttcttcacctacttctattgggaggtgtggggcaaaggcacccaggtgacagtgtctagcggcggagtgggcggcgagggcggcggtggctccggcagtgacatcagcgtggctcccggcgaaacagtgcggatctcttgtggcggagaaagcattggatctagagccgtgcagtggtaccagcacagagccggccaggccccaaagctgatcatctacaacaaccaggatagacctcctggcatccccgagcggttcagcggcagccctgacatcgacttcggcaccaccgccaccctgaccatcaccaatgtggaagccggcgatgaggccacatactattgccacatctgggactccagacggcccaccaactgggtctttggcggaggaacaaccctgacagtgctgggccaaggcggaagcggcgggtccggcggctctggaggcagcggcggctctggcggaagcggaggctctggcggcagcggcggcagcggcggcgacaagacccacacctgtcccccttgccccgctcccgagctgctgggaggcccctccgtgttcctgttccccccaaagcccaaagataccctgatgatcagcaggaccccggaggtgacctgcgtggtggtggacgtgagccacgaggaccctgaggtgaagttcaactggtacgtggacggcgtggaggttcacaacgccaagaccaaacccagggaggagcagtacaacagcacttacagggtggtgagcgtgctgaccgtgctgcaccaggactggctgaacggcaaggagtacaaatgcaaggttagcaacaaggcccttccagcccccatcgagaagaccatcagcaaggccaagggacagcctagggagccccaggtctacactctgccccccagccgcgacgagctgaccaagaaccaggtgagtttgtggtgtctggtgaagggcttctaccccagcgacatcgccgtggagtgggagagcaacggccagcccgagaacaactacaaaaccaccccccctgtgctggactcagacggcagctttttcctgtattctaagctcaccgtggacaagagcaggtggcagcagggcaacgtgttcagttgcagcgtgatgcacgaggccctgcacaaccactacacccagaagagcttgagcctgagccccggaaag

SEQ ID NO: 83 - DNA, 1371 bp, synthetic construct
  source 1..1371: mol_type = other DNA
gctgatctggtgcagagcggcgccgtggtgaagaaaccaggcgacagcgtgcgcatcagctgtgaagcccagggctatagattccccgactacatcatccactggatcaggcgggcccctggccagggccctgaatggatgggctggatgaaccccatgggaggacaagtgaacattccttggaagttccagggaagagtgtccatgaccagagataccagcatcgagacagcttttctggacctgagaggcctgaagtccgacgacaccgccgtctactactgcgtgcgggacagaagcaacggctctggcaagcggttcgagagcagcaattggttcctggatctgtggggcagaggcacagccgtgaccatccagtctgccagcaccaagggtcccagcgtattccccctggccccaagtagcaaaagcaccagtggtggcacagcggccctcggatgcctggttaaggactatttccccgagccggtaaccgtaagctggaacagtggagcactgaccagcggcgtgcataccttccccgctgtactgcaatcctccggcctgtactcactgagcagcgtagtgacggtgccctctagcagcctgggcacccagacctacatctgcaacgtgaaccataaaccgtctaacacgaaggtcgacaaaaaagttgagcctaaaagctgcgacaagacccacacctgtcccccttgccccgctcccgagctgctgggaggcccctccgtgttcctgttccccccaaagcccaaagataccctgatgatcagcaggaccccggaggtgacctgcgtggtggtggacgtgagccacgaggaccctgaggtgaagttcaactggtacgtggacggcgtggaggttcacaacgccaagaccaaacccagggaggagcagtacaacagcacttacagggtggtgagcgtgctgaccgtgctgcaccaggactggctgaacggcaaggagtacaaatgcaaggttagcaacaaggcccttccagcccccatcgagaagaccatcagcaaggccaagggacagcctagggagccccaggtctacactctgccccccagccgcgacgagctgaccaagaaccaggtgagtttgtcttgtgctgtgaagggcttctaccccagcgacatcgccgtggagtgggagagcaacggccagcccgagaacaactacaaaaccaccccccctgtgctggactcagacggcagctttttcctggtctctaagctcaccgtggacaagagcaggtggcagcagggcaacgtgttcagttgcagcgtgatgcacgaggccctgcacaaccactacacccagaagagcttgagcctgagccccggaaag

SEQ ID NO: 84 - DNA, 609 bp, synthetic construct
  source 1..609: mol_type = other DNA
gcactgacccagcctaggagcgtgtccgcctctccaggacagagcgtgacaatctcctgcaccggcacacacaacctggtgtcctggtgtcagcaccagccaggacgggcccctaagctgctgatctacgacttcaataagcggccatctggcgtgcccgatcggttcagcggctccggctctggaggcaccgccagcctgaccatcacaggactgcaggacgatgacgatgccgagtacttctgctgggcctatgaggcctttggcggaggcaccaagctgacagtgctgggacagcctaaggccgctcctagcgttacactgttccctccaagctctgaggaactgcaggccaacaaagccacactcgtgtgcctgatcagcgacttttatcctggcgccgtgacagtggcctggaaggctgatagttctcctgtgaaggccggcgtggaaaccaccacacctagcaagcagtccaacaacaaatacgccgccagcagctacctgagcctgacacctgaacagtggaagtcccacaagtcctacagctgccaagtgacccacgagggcagcaccgtggaaaaaacagtggcccctacagagtgcggc

SEQ ID NO: 85 - DNA, 1527 bp, synthetic construct
  source 1..1527: mol_type = other DNA
cagatgcagctgcaggagtctggccctggactggtgaagcccggcgaaaccctgagcctcacatgcagcgtgagcggagcctctatcagcagcagctactggagctggctgcgggaaacccccggaaagggactggaatggatcggctatacacaccacagcggagataccaactacgccccttctctgaaaagccgggtgcacctgggcctgcaccccagcaagaaccaagtgtccctgtctctgaccagcgtgaccgccgctgatacagccgtgtactactgtgctagaacactgcatggcagaagaatctacggcgtggtggcctttaacgagttcttcacctacttctactgggaggtgtggggcaagggcacacaggtgaccgtgtctagcggcagcgtgggctctgtgtccggctcaggcggcggaggcgccagcgacatctccgtcgcccctggcgagaccgttagaatcagctgcggcggagagagcattggctccagagccgtgcagtggtaccagcaccgggccggccaggccccaaaactgatcatctacaacaaccaggacagacctccaggcatccccgagagattcagcggatcccctgacatcgacttcggcaccaccgccacactgacaatcaccaatgtggaagctggcgacgaggccacctattactgccacatctgggacagcaggcggcctaccaattgggtctttggcggcggtacaaccctgaccgtgctgggacaaggcggatctggcggcagcggcggatctggaggcagcggcggcagcggcggcagcggcggcagcggcggtagcggaggcagcggcggcgacaagacccacacctgtcccccttgccccgctcccgagctgctgggaggcccctccgtgttcctgttccccccaaagcccaaagataccctgatgatcagcaggaccccggaggtgacctgcgtggtggtggacgtgagccacgaggaccctgaggtgaagttcaactggtacgtggacggcgtggaggttcacaacgccaagaccaaacccagggaggagcagtacaacagcacttacagggtggtgagcgtgctgaccgtgctgcaccaggactggctgaacggcaaggagtacaaatgcaaggttagcaacaaggcccttccagcccccatcgagaagaccatcagcaaggccaagggacagcctagggagccccaggtctacactctgccccccagccgcgacgagctgaccaagaaccaggtgagtttgtggtgtctggtgaagggcttctaccccagcgacatcgccgtggagtgggagagcaacggccagcccgagaacaactacaaaaccaccccccctgtgctggactcagacggcagctttttcctgtattctaagctcaccgtggacaagagcaggtggcagcagggcaacgtgttcagttgcagcgtgatgcacgaggccctgcacaaccactacacccagaagagcttgagcctgagccccggaaag

SEQ ID NO: 86 - DNA, 1371 bp, synthetic construct; identical to SEQ ID NO: 83
  source 1..1371: mol_type = other DNA

SEQ ID NO: 87 - DNA, 609 bp, synthetic construct; identical to SEQ ID NO: 84
  source 1..609: mol_type = other DNA

SEQ ID NO: 88 - DNA, 1521 bp, synthetic construct; identical to SEQ ID NO: 82
  source 1..1521: mol_type = other DNA

SEQ ID NO: 89 - DNA, 1392 bp, synthetic construct
  source 1..1392: mol_type = other DNA
gctgatctggtgcagtctggcgccgtggtgaagaaacctggcgacagcgtgcggatttcttgtgaagcccagggttatagattccccgactacatcatccactggatcagaagagcccctggacagggacctgagtggatgggctggatgaaccccatgggcggccaagtgaacatcccttggaagttccagggcagagtgtccatgaccagacagctgagccaggacccagatgaccctgattggggcaccgcttttctggacctgagaggcctgaagtccgacgacaccgccgtctactactgcgtgcgggacagaagcaacggcagcggcaagcggttcgagagcagcaattggttcctggatctgtggggaaggggcacagccgttacaatccagagcgccagcaccaagggtcccagcgtattccccctggccccaagtagcaaaagcaccagtggtggcacagcggccctcggatgcctggttaaggactatttccccgagccggtaaccgtaagctggaacagtggagcactgaccagcggcgtgcataccttccccgctgtactgcaatcctccggcctgtactcactgagcagcgtagtgacggtgccctctagcagcctgggcacccagacctacatctgcaacgtgaaccataaaccgtctaacacgaaggtcgacaaaaaagttgagcctaaaagctgcgacaagacccacacctgtcccccttgccccgctcccgagctgctgggaggcccctccgtgttcctgttccccccaaagcccaaagataccctgatgatcagcaggaccccggaggtgacctgcgtggtggtggacgtgagccacgaggaccctgaggtgaagttcaactggtacgtggacggcgtggaggttcacaacgccaagaccaaacccagggaggagcagtacaacagcacttacagggtggtgagcgtgctgaccgtgctgcaccaggactggctgaacggcaaggagtacaaatgcaaggttagcaacaaggcccttccagcccccatcgagaagaccatcagcaaggccaagggacagcctagggagccccaggtctacactctgccccccagccgcgacgagctgaccaagaaccaggtgagtttgtcttgtgctgtgaagggcttctaccccagcgacatcgccgtggagtgggagagcaacggccagcccgagaacaactacaaaaccaccccccctgtgctggactcagacggcagctttttcctggtctctaagctcaccgtggacaagagcaggtggcagcagggcaacgtgttcagttgcagcgtgatgcacgaggccctgcacaaccactacacccagaagagcttgagcctgagccccggaaag

SEQ ID NO: 90 - DNA, 609 bp, synthetic construct; identical to SEQ ID NO: 84
  source 1..609: mol_type = other DNA

SEQ ID NO: 91 - DNA, 1527 bp, synthetic construct; identical to SEQ ID NO: 85
  source 1..1527: mol_type = other DNA

SEQ ID NO: 92 - DNA, 1392 bp, synthetic construct; identical to SEQ ID NO: 89
  source 1..1392: mol_type = other DNA

SEQ ID NO: 93 - DNA, 609 bp, synthetic construct; identical to SEQ ID NO: 84
  source 1..609: mol_type = other DNA

SEQ ID NO: 94 - DNA, 1572 bp, synthetic construct
  source 1..1572: mol_type = other DNA
caggcccagctggtgcagagcggacctgaggtgcggaagcctggcacaagcgtcaaggtgagctgcaaaaaccccggcaacacactgacaacatacgacctgcactgggtgcggcaggtgcctggccaaggactgcagtggatgggctggatcagccacgagggcgacaagaaagtcatcgtggaaagattcaaggccaaggtgacaatcgactgggacagaagcacaaacaccgcctacctgcaactgagcggcctgaccagcgaggataccgctgtgtattactgcgccaaaggctctaagcatagactgcgggactacgccctgtacgacgaggacggcgccctgaattgggccgtggacgttgattacctcgctaatctggagttctggggccagggcaccgccgtgaccgtgagcagcggcggcggtggctccggcggcgggggcagcggcggaggcggaagcgacatcgtgctgacccagtctccactgagcctgtctgtgactcctggcgagtccgccagcatcagctgcaagagcagccagagcctgatccacggcgatagaaacaactacctggcctggtacgtgcagaagcccggcagaagccctcagctgctgatcgatctggcttctgagagagcctctggagtgcccgaccggtttagcggcagcggatctagaaaggacttcaccctgaagatcagcagagtggaaaccgaggatgtgggcacctattactgtatgcagggcagggaaagcccttggaccttcggccagggcaccaaggtggatattaagggcggcagcggcggaagcggaggctccggcggatccggtggcagcggcggctctggcggctcaggcggctccggaggctctggcggcgacaagacccacacctgtcccccttgccccgctcccgagctgctgggaggcccctccgtgttcctgttccccccaaagcccaaagataccctgatgatcagcaggaccccggaggtgacctgcgtggtggtggacgtgagccacgaggaccctgaggtgaagttcaactggtacgtggacggcgtggaggttcacaacgccaagaccaaacccagggaggagcagtacaacagcacttacagggtggtgagcgtgctgaccgtgctgcaccaggactggctgaacggcaaggagtacaaatgcaaggttagcaacaaggcccttccagcccccatcgagaagaccatcagcaaggccaagggacagcctagggagccccaggtctacactctgccccccagccgcgacgagctgaccaagaaccaggtgagtttgtggtgtctggtgaagggcttctaccccagcgacatcgccgtggagtgggagagcaacggccagcccgagaacaactacaaaaccaccccccctgtgctggactcagacggcagctttttcctgtattctaagctcaccgtggacaagagcaggtggcagcagggcaacgtgttcagttgcagcgtgatgcacgaggccctgcacaaccactacacccagaagagcttgagcctgagccccggaaag

SEQ ID NO: 95 - DNA, 1371 bp, synthetic construct; identical to SEQ ID NO: 83
  source 1..1371: mol_type = other DNA

SEQ ID NO: 96 - DNA, 609 bp, synthetic construct; identical to SEQ ID NO: 84
  source 1..609: mol_type = other DNA

SEQ ID NO: 97 - DNA, 1572 bp, synthetic construct
  source 1..1572: mol_type = other DNA
gacatcgtgctgacccagagccctctgagcctgagcgtgacccctggcgagagcgcctccatcagctgcaagagcagccagtctctgatccacggcgaccggaacaattacctcgcttggtacgtgcagaagcctggaagaagcccccagctgctgattgacctggcttctgagagagccagcggcgtgcctgataggttcagcggcagcggctcccggaaggacttcaccctgaagatcagcagagtggaaaccgaggatgtgggcacctactattgtatgcagggcagagagtctccatggaccttcggccagggaacaaaggtggacatcaagggcggcggcggcagcggcggcggcggatccggaggaggaggcagccaggcccagctggtgcagagcggacctgaggtgcggaaacctggcaccagcgtgaaggtttcctgcaagaaccccggcaataccctgacaacctacgacctgcattgggtgcgccaggtgcccggacaaggcctgcagtggatgggctggatcagccacgagggcgataagaaagtgatcgtggaaagatttaaggccaaagtcacaatcgactgggaccggagcaccaacacagcctacctgcaactgagcggtcttacatctgaagacaccgctgtctactactgcgccaagggctcaaagcacagactgagagattacgccctgtacgacgaggacggcgccctgaactgggccgtggatgtggactacctggccaacctggagttctggggacagggcacagccgtgaccgtgtccagtggcggcagcggcggcagcggcggctctggcggatctggtggctctggcggaagcggcggcagcggcggctccggaggcagcggcggcgacaagacccacacctgtcccccttgccccgctcccgagctgctgggaggcccctccgtgttcctgttccccccaaagcccaaagataccctgatgatcagcaggaccccggaggtgacctgcgtggtggtggacgtgagccacgaggaccctgaggtgaagttcaactggtacgtggacggcgtggaggttcacaacgccaagaccaaacccagggaggagcagtacaacagcacttacagggtggtgagcgtgctgaccgtgctgcaccaggactggctgaacggcaaggagtacaaatgcaaggttagcaacaaggcccttccagcccccatcgagaagaccatcagcaaggccaagggacagcctagggagccccaggtctacactctgccccccagccgcgacgagctgaccaagaaccaggtgagtttgtggtgtctggtgaagggcttctaccccagcgacatcgccgtggagtgggagagcaacggccagcccgagaacaactacaaaaccaccccccctgtgctggactcagacggcagctttttcctgtattctaagctcaccgtggacaagagcaggtggcagcagggcaacgtgttcagttgcagcgtgatgcacgaggccctgcacaaccactacacccagaagagcttgagcctgagccccggaaag

SEQ ID NO: 98 - DNA, 1371 bp, synthetic construct; identical to SEQ ID NO: 83
  source 1..1371: mol_type = other DNA

SEQ ID NO: 99 - DNA, 609 bp, synthetic construct; identical to SEQ ID NO: 84
  source 1..609: mol_type = other DNA

SEQ ID NO: 100 - DNA, 1572 bp, synthetic construct; identical to SEQ ID NO: 94
  source 1..1572: mol_type = other DNA

SEQ ID NO: 101 - DNA, 1392 bp, synthetic construct; identical to SEQ ID NO: 89
  source 1..1392: mol_type = other DNA

SEQ ID NO: 102 - DNA, 609 bp, synthetic construct; identical to SEQ ID NO: 84
  source 1..609: mol_type = other DNA

SEQ ID NO: 103 - DNA, 1572 bp, synthetic construct; identical to SEQ ID NO: 97
  source 1..1572: mol_type = other DNA

SEQ ID NO: 104 - DNA, 1392 bp, synthetic construct; identical to SEQ ID NO: 89
  source 1..1392: mol_type = other DNA

SEQ ID NO: 105 - DNA, 609 bp, synthetic construct; identical to SEQ ID NO: 84
  source 1..609: mol_type = other DNA

SEQ ID NO: 106 - DNA, 1521 bp, synthetic construct; identical to SEQ ID NO: 82
  source 1..1521: mol_type = other DNA

SEQ ID NO: 107 - DNA, 1572 bp, synthetic construct
  source 1..1572: mol_type = other DNA
caggcccagctggtgcagagcggacctgaggtgcggaagcctggcacaagcgtcaaggtgagctgcaaaaaccccggcaacacactgacaacatacgacctgcactgggtgcggcaggtgcctggccaaggactgcagtggatgggctggatcagccacgagggcgacaagaaagtcatcgtggaaagattcaaggccaaggtgacaatcgactgggacagaagcacaaacaccgcctacctgcaactgagcggcctgaccagcgaggataccgctgtgtattactgcgccaaaggctctaagcatagactgcgggactacgccctgtacgacgaggacggcgccctgaattgggccgtggacgttgattacctcgctaatctggagttctggggccagggcaccgccgtgaccgtgagcagcggcggcggtggctccggcggcgggggcagcggcggaggcggaagcgacatcgtgctgacccagtctccactgagcctgtctgtgactcctggcgagtccgccagcatcagctgcaagagcagccagagcctgatccacggcgatagaaacaactacctggcctggtacgtgcagaagcccggcagaagccctcagctgctgatcgatctggcttctgagagagcctctggagtgcccgaccggtttagcggcagcggatctagaaaggacttcaccctgaagatcagcagagtggaaaccgaggatgtgggcacctattactgtatgcagggcagggaaagcccttggaccttcggccagggcaccaaggtggatattaagggcggcagcggcggaagcggaggctccggcggatccggtggcagcggcggctctggcggctcaggcggctccggaggctctggcggcgacaagacccacacctgtcccccttgccccgctcccgagctgctgggaggcccctccgtgttcctgttccccccaaagcccaaagataccctgatgatcagcaggaccccggaggtgacctgcgtggtggtggacgtgagccacgaggaccctgaggtgaagttcaactggtacgtggacggcgtggaggttcacaacgccaagaccaaacccagggaggagcagtacaacagcacttacagggtggtgagcgtgctgaccgtgctgcaccaggactggctgaacggcaaggagtacaaatgcaaggttagcaacaaggcccttccagcccccatcgagaagaccatcagcaaggccaagggacagcctagggagccccaggtctacactctgccccccagccgcgacgagctgaccaagaaccaggtgagtttgtcttgtgctgtgaagggcttctaccccagcgacatcgccgtggagtgggagagcaacggccagcccgagaacaactacaaaaccaccccccctgtgctggactcagacggcagctttttcctggtctctaagctcaccgtggacaagagcaggtggcagcagggcaacgtgttcagttgcagcgtgatgcacgaggccctgcacaaccactacacccagaagagcttgagcctgagccccggaaag

SEQ ID NO: 108 - DNA, 1527 bp, synthetic construct; identical to SEQ ID NO: 85
  source 1..1527: mol_type = other DNA

SEQ ID NO: 109 - DNA, 1572 bp, synthetic construct; identical to SEQ ID NO: 107
  source 1..1572: mol_type = other DNA

SEQ ID NO: 110 - DNA, 1521 bp, synthetic construct; identical to SEQ ID NO: 82
  source 1..1521: mol_type = other DNA

SEQ ID NO: 111 - DNA, 1572 bp, synthetic construct
  source 1..1572: mol_type = other DNA
gacatcgtgctgacccagagccctctgagcctgagcgtgacccctggcgagagcgcctccatcagctgcaagagcagccagtctctgatccacggcgaccggaacaattacctcgcttggtacgtgcagaagcctggaagaagcccccagctgctgattgacctggcttctgagagagccagcggcgtgcctgataggttcagcggcagcggctcccggaaggacttcaccctgaagatcagcagagtggaaaccgaggatgtgggcacctactattgtatgcagggcagagagtctccatggaccttcggccagggaacaaaggtggacatcaagggcggcggcggcagcggcggcggcggatccggaggaggaggcagccaggcccagctggtgcagagcggacctgaggtgcggaaacctggcaccagcgtgaaggtttcctgcaagaaccccggcaataccctgacaacctacgacctgcattgggtgcgccaggtgcccggacaaggcctgcagtggatgggctggatcagccacgagggcgataagaaagtgatcgtggaaagatttaaggccaaagtcacaatcgactgggaccggagcaccaacacagcctacctgcaactgagcggtcttacatctgaagacaccgctgtctactactgcgccaagggctcaaagcacagactgagagattacgccctgtacgacgaggacggcgccctgaactgggccgtggatgtggactacctggccaacctggagttctggggacagggcacagccgtgaccgtgtccagtggcggcagcggcggcagcggcggctctggcggatctggtggctctggcggaagcggcggcagcggcggctccggaggcagcggcggcgacaagacccacacctgtcccccttgccccgctcccgagctgctgggaggcccctccgtgttcctgttccccccaaagcccaaagataccctgatgatcagcaggaccccggaggtgacctgcgtggtggtggacgtgagccacgaggaccctgaggtgaagttcaactggtacgtggacggcgtggaggttcacaacgccaagaccaaacccagggaggagcagtacaacagcacttacagggtggtgagcgtgctgaccgtgctgcaccaggactggctgaacggcaaggagtacaaatgcaaggttagcaacaaggcccttccagcccccatcgagaagaccatcagcaaggccaagggacagcctagggagccccaggtctacactctgccccccagccgcgacgagctgaccaagaaccaggtgagtttgtcttgtgctgtgaagggcttctaccccagcgacatcgccgtggagtgggagagcaacggccagcccgagaacaactacaaaaccaccccccctgtgctggactcagacggcagctttttcctggtctctaagctcaccgtggacaagagcaggtggcagcagggcaacgtgttcagttgcagcgtgatgcacgaggccctgcacaaccactacacccagaagagcttgagcctgagccccggaaag

SEQ ID NO: 112 - DNA, 1527 bp, synthetic construct; identical to SEQ ID NO: 85
  source 1..1527: mol_type = other DNA

SEQ ID NO: 113 - DNA, 1572 bp, synthetic construct; identical to SEQ ID NO: 111
  source 1..1572: mol_type = other DNA

SEQ ID NO: 114 - DNA, 1386 bp, synthetic construct
  source 1..1386: mol_type = other DNA
cagatgcagctgcaggagagcggaccaggactggtgaagcctggcgagaccctgtccctgacatgctctgtgagcggcgcctctatcagctcctcttactggagctggctgagggagacccctggcaagggcctggagtggatcggctacacccaccactctggcgacacaaactatgccccaagcctgaagtcccgcgtgcacctgggactgcacccatccaagaatcaggtgtccctgtctctgacctctgtgacagccgccgataccgccgtgtactattgtgcccggacactgcacggccggagaatctatggcgtggtggccttcaacgagtactatacctacttttattggcccacatggggcaagggcacccaggtgaccgtgagcagcgcctctacaaagggcccctccgtttttccactggctcctagcagcaagagcacatctggtggaacagccgctctgggctgcctggtcaaggattactttcctgagcctgtgaccgtgtcctggaattctggtgctctgacaagcggcgtgcacacctttccagccgtgctgcaaagcagcggcctgtactctctgtctagcgtggtcacagtgcctagctctagcctgggcacccagacctacatctgcaacgtgaaccacaagcctagcaacaccaaggtggacaagaaggtggaacccaagagctgcgacaagacccacacctgtcctccatgtcctgctccagaactgctcggcggaccctccgtgtttctgttccctccaaagcctaaggacaccctgatgatcagcagaacccctgaagtgacctgcgtggtggtggatgtgtcccacgaggacccagaagtgaagttcaattggtacgtggacggcgtggaagtgcacaacgccaagaccaagccaagagaggaacagtacaacagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagcctagagaaccccaggtttacacactgcctccaagcagggacgagctgaccaagaatcaggtgtccctgacctgcctcgtgaagggcttctacccttccgatatcgccgtggaatgggagagcaacggacagcccgagaacaactacaagacaacccctcctgtgctggacagcgacggctcattcttcctgtacagcaagctgaccgtggacaagtccagatggcagcagggcaatgtgttcagctgctctgtgctgcacgaggccctgcactcccactacacccagaaaagcctgtctctgagccccggcaag

SEQ ID NO: 115 - DNA, 1428 bp, synthetic construct
  source 1..1428: mol_type = other DNA
gccctgacacagcccagaagcgtgtctgcctctcctggccagagcgtgaccatcagctgcaccggcacacacaacctggtcagctggtgccagcatcagcctggccgcgccccaaagctgctgatctacgacttcaacaagcggcccagcggcgtgcccgacagattcagcggcagcggctccggcggaaccgccagcctgaccatcacaggcctgcaggatgacgacgacgccgagtacttctgctgggcctacgaggctttcggcggcggaaccaagctgaccgtactgggccaaggcggaggaggcagcggagggggcggcagcggcggaggaggctctgctgatctcgtgcagtctggcgccgtggtgaagaaacccggggattccgttagaatttcctgcgaggcccagggctacagatttccagactacatcatccactggatccggcgggctcctggccagggccctgaatggatgggctggatgaaccccatgggaggccaagtgaacatcccctggaagttccagggaagggtgtccatgaccagagatactagcatcgaaaccgccttcctggacctgaggggcctgaaaagcgacgataccgccgtctactattgtgtgcgggacagaagcaatggctctggcaagagattcgagagcagcaattggttcctggatctgtggggcagaggcaccgccgtgaccattcagagcggaggcggcggctctggaggaggcggcagcggaggcggcggctcaggcggcggcggcagcggaggcggcggctctgacatcagcgtggcccctggcgagacagtgcggatcacctgtggcggcgaaagcatcggctctagagccgtgcagtggtaccagcacagacctggccaggcccctagactgatcatctacaacaaccaggacagacctcctggcatccctgaaagatttagcggcagccctgacatcgactttggaacaacagctacactgaccatcagcaacgtggaagccggcgacgaggccacatactactgccacatctgggacagcagacggccaacaaactgggagctgggacctggcacaaccctgaccgtgctgggccagcccaaggccgctcccagcgtgaccctgttcccccccagcagcgaggagctgcaggccaacaaggccaccctggtgtgcctgatctcagacttctatcctggggcagttaccgtggcctggaaagccgacagcagccccgtgaaggccggagtggagacaaccaccccaagcaagcagagcaacaacaagtacgccgcctccagctacctgagcctgacccccgagcagtggaagagccacaagagctacagctgccaggtgacccacgagggcagcaccgtggagaagaccgttgcacccaccgagtgcagc

SEQ ID NO: 116 - DNA, 1386 bp, synthetic construct; identical to SEQ ID NO: 114
  source 1..1386: mol_type = other DNA

SEQ ID NO: 117 - DNA, 1449 bp, synthetic construct
  source 1..1449: mol_type = other DNA
gccctgacccagcctcgtagcgtgtccgccagccctggacagtctgtgaccatctcctgcaccggcacccacaacctggtcagctggtgccagcaccagcccggcagagctccaaagctgctgatctacgacttcaacaagagacccagcggcgtgcccgacagattcagcggcagcggaagcggcggtacagcctctctgaccattacaggcctgcaggacgacgatgacgccgagtacttctgctgggcttacgaggcttttggcggaggaaccaagctgacagtgctgggacagggcggcggaggcagcggcggaggcggtagtggcggcggtggcagcgccgacctggtgcagagcggcgccgtggtgaaaaagcctggagatagcgtgagaatctcttgtgaagcccaggggtacagattccctgactacatcatccactggatcagaagggccccaggccagggacctgagtggatgggctggatgaaccccatgggcggccaagtgaacatcccctggaagttccagggcagagtgtctatgaccagacaactgagccaggatcccgatgatcctgattggggcacagccttcctggacctgagaggcctgaaaagcgacgacaccgctgtttactattgtgtgcgcgacagaagcaatggctctggcaagcggttcgagagctctaattggttcctggatctgtggggaagaggaaccgccgtgacaatccagagcggcggcggcggcagcggcgggggcggctccggcggcggcggtagcggcggaggcggctccggaggcggcggctctgacatcagcgtcgcccctggagagacagtgcggattacctgcggcggcgaaagcatcggctctcgggccgtgcagtggtaccagcatagacccggccaggcccctcggctgatcatctacaacaaccaggacagaccacctggcatccctgaaagattcagcggctcccctgacatcgacttcggcactaccgcaaccctgaccatcagcaacgtggaagccggcgacgaggccacatactactgccacatctgggacagcagacggcctacaaactgggagctgggccctggcaccaccctgaccgtgctcggccaacccaaggccgctcccagcgtgaccctgttcccccccagcagcgaggagctgcaggccaacaaggccaccctggtgtgcctgatctcagacttctatcctggggcagttaccgtggcctggaaagccgacagcagccccgtgaaggccggagtggagacaaccaccccaagcaagcagagcaacaacaagtacgccgcctccagctacctgagcctgacccccgagcagtggaagagccacaagagctacagctgccaggtgacccacgagggcagcaccgtggagaagaccgttgcacccaccgagtgcagc

SEQ ID NO: 118 - DNA, 1572 bp, synthetic construct; identical to SEQ ID NO: 94
  source 1..1572: mol_type = other DNA

SEQ ID NO: 119 - DNA, 1386 bp, synthetic construct
  source 1..1386: mol_type = other DNA
caaatgcagctgcaggagagcggacctggcctggtgaagcccggcgagacactgagcctgacatgtagcgtgtctggcgccagcatcagcagcagctattggtcttggctgcgggaaacccctggcaagggcctggaatggatcggctacacccaccacagcggagataccaattacgccccatctctgaagagcagagtgcacctgggcctccatcctagcaagaaccaggtgtccctgtctctgaccagcgttacagccgctgacaccgccgtgtactactgcgccagaaccctgcacggcagacggatctacggcgtggtggcttttaacgagttcttcacctacttctactgggaggtgtggggaaaaggcacccaggtcacagtgtccagcgccagcaccaagggtcccagcgtattccccctggccccaagtagcaaaagcaccagtggtggcacagcggccctcggatgcctggttaaggactatttccccgagccggtaaccgtaagctggaacagtggagcactgaccagcggcgtgcataccttccccgctgtactgcaatcctccggcctgtactcactgagcagcgtagtgacggtgccctctagcagcctgggcacccagacctacatctgcaacgtgaaccataaaccgtctaacacgaaggtcgacaaaaaagttgagcctaaaagctgcgacaagacccacacctgtcccccttgccccgctcccgagctgctgggaggcccctccgtgttcctgttccccccaaagcccaaagataccctgatgatcagcaggaccccggaggtgacctgcgtggtggtggacgtgagccacgaggaccctgaggtgaagttcaactggtacgtggacggcgtggaggttcacaacgccaagaccaaacccagggaggagcagtacaacagcacttacagggtggtgagcgtgctgaccgtgctgcaccaggactggctgaacggcaaggagtacaaatgcaaggttagcaacaaggcccttccagcccccatcgagaagaccatcagcaaggccaagggacagcctagggagccccaggtctacactctgccccccagccgcgacgagctgaccaagaaccaggtgagtttgtcttgtgctgtgaagggcttctaccccagcgacatcgccgtggagtgggagagcaacggccagcccgagaacaactacaaaaccaccccccctgtgctggactcagacggcagctttttcctggtctctaagctcaccgtggacaagagcaggtggcagcagggcaacgtgttcagttgcagcgtgatgcacgaggccctgcacaaccactacacccagaagagcttgagcctgagccccggaaag

SEQ ID NO: 120 - DNA, 1479 bp, synthetic construct
  source 1..1479: mol_type = other DNA
gccctgacccagcctcgtagcgtgtccgccagccctggacagtctgtgaccatctcctgcaccggcacccacaacctggtcagctggtgccagcaccagcccggcagagctccaaagctgctgatctacgacttcaacaagagacccagcggcgtgcccgacagattcagcggcagcggaagcggcggtacagcctctctgaccattacaggcctgcaggacgacgatgacgccgagtacttctgctgggcttacgaggcttttggcggaggaaccaagctgacagtgctgggacagggcggcggaggcagcggcggaggcggtagtggcggcggtggcagcgccgacctggtgcagagcggcgccgtggtgaaaaagcctggagatagcgtgagaatctcttgtgaagcccaggggtacagattccctgactacatcatccactggatcagaagggccccaggccagggacctgagtggatgggctggatgaaccccatgggcggccaagtgaacatcccctggaagttccagggcagagtgtctatgaccagacaactgagccaggatcccgatgatcctgattggggcacagccttcctggacctgagaggcctgaaaagcgacgacaccgctgtttactattgtgtgcgcgacagaagcaatggctctggcaagcggttcgagagctctaattggttcctggatctgtggggaagaggaaccgccgtgacaatccagagcggcggcggcggcagcggcgggggcggctccggcggcggcggcagcggcgggggcggctccggcggcggcggtagcggcggaggcggctccggaggcggcggctctgacatcagcgtcgcccctggagagacagtgcggattacctgcggcggcgaaagcatcggctctcgggccgtgcagtggtaccagcatagacccggccaggcccctcggctgatcatctacaacaaccaggacagaccacctggcatccctgaaagattcagcggctcccctgacatcgacttcggcactaccgcaaccctgaccatcagcaacgtggaagccggcgacgaggccacatactactgccacatctgggacagcagacggcctacaaactgggagctgggccctggcaccaccctgaccgtgctcggccaacccaaggccgctcccagcgtgaccctgttcccccccagcagcgaggagctgcaggccaacaaggccaccctggtgtgcctgatctcagacttctatcctggggcagttaccgtggcctggaaagccgacagcagccccgtgaaggccggagtggagacaaccaccccaagcaagcagagcaacaacaagtacgccgcctccagctacctgagcctgacccccgagcagtggaagagccacaagagctacagctgccaggtgacccacgagggcagcaccgtggagaagaccgttgcacccaccgagtgcagc

SEQ ID NO: 121 - DNA, 1572 bp, synthetic construct; identical to SEQ ID NO: 97
  source 1..1572: mol_type = other DNA

SEQ ID NO: 122 - DNA, 1386 bp, synthetic construct; identical to SEQ ID NO: 119
  source 1..1386: mol_type = other DNA

SEQ ID NO: 123 - DNA, 1479 bp, synthetic construct; identical to SEQ ID NO: 120
  source 1..1479: mol_type = other DNA

SEQ ID NO: 124 - DNA, 2370 bp, synthetic construct
  source 1..2370: mol_type = other DNA
caaatgcagctgcaggagagcggtcctggcctggtgaagcctggcgagacactgagcctgacatgcagcgtgtccggcgccagcatcagcagcagctactggagctggctgcgggaaacccctggaaaaggcctggaatggatcggctacacccaccacagcggcgacacaaattacgccccaagcctcaagtccagagtgcacctgggcctgcaccccagcaagaaccaggtgtccctgtctctgaccagcgtcaccgcagccgacaccgctgtgtactactgcgccagaaccctgcatggcagaaggatctacggcgttgtggcctttaacgagttcttcacctacttctattgggaggtgtggggcaaaggcacccaggtgacagtgtctagcggcggagtgggcggcgagggcggcggtggctccggcagtgacatcagcgtggctcccggcgaaacagtgcggatctcttgtggcggagaaagcattggatctagagccgtgcagtggtaccagcacagagccggccaggccccaaagctgatcatctacaacaaccaggatagacctcctggcatccccgagcggttcagcggcagccctgacatcgacttcggcaccaccgccaccctgaccatcaccaatgtggaagccggcgatgaggccacatactattgccacatctgggactccagacggcccaccaactgggtctttggcggaggaacaaccctgacagtgctgggccaaggcggaagcggcgggtccggcggctctggaggcagcggcggctctggcggaagcggaggctctggcggcagcggcggcagcggcggcgacaagacccacacctgtcccccttgccccgctcccgagctgctgggaggcccctccgtgttcctgttccccccaaagcccaaagataccctgatgatcagcaggaccccggaggtgacctgcgtggtggtggacgtgagccacgaggaccctgaggtgaagttcaactggtacgtggacggcgtggaggttcacaacgccaagaccaaacccagggaggagcagtacaacagcacttacagggtggtgagcgtgctgaccgtgctgcaccaggactggctgaacggcaaggagtacaaatgcaaggttagcaacaaggcccttccagcccccatcgagaagaccatcagcaaggccaagggacagcctagggagccccaggtctacactctgccccccagccgcgacgagctgaccaagaaccaggtgagtttgtggtgtctggtgaagggcttctaccccagcgacatcgccgtggagtgggagagcaacggccagcccgagaacaactacaaaaccaccccccctgtgctggactcagacggcagctttttcctgtattctaagctcaccgtggacaagagcaggtggcagcagggcaacgtgttcagttgcagcgtgatgcacgaggccctgcacaaccactacacccagaagagcttgagcctgagccccggaaagggcggcggtggctccggcggcgggggcagcggcggaggcggaagccaggcccagctggtgcagagcggacctgaggtgcggaagcctggcacaagcgtcaaggtgagctgcaaaaaccccggcaacacactgacaacatacgacctgcactgggtgcggcaggtgcctggccaaggactgcagtggatgggctggatcagccacgagggcgacaagaaagtcatcgtggaaagattcaaggccaaggtgacaatcgactgggacagaagcacaaacaccgcctacctgcaactgagcggcctgaccagcgaggataccgctgtgtattactgcgccaaaggctctaagcatagactgcgggactacgccctgtacgacgaggacggcgccctgaattgggccgtggacgttgattacctcgctaatctggagttctggggccagggcaccgccgtgaccgtgagcagcggcggcggtggctccggcggcgggggcagcggcggaggcggaagcgacatcgtgctgacccagtctccactgagcctgtctgtgactcctggcgagtccgccagcatcagctgcaagagcagccagagcctgatccacggcgatagaaacaactacctggcctggtacgtgcagaagcccggcagaagccctcagctgctgatcgatctggcttctgagagagcctctggagtgcccgaccggtttagcggcagcggatctagaaaggacttcaccctgaagatcagcagagtggaaaccgaggatgtgggcacctattactgtatgcagggcagggaaagcccttggaccttcggccagggcaccaaggtggatattaag

SEQ ID NO: 125 - DNA, 1392 bp, synthetic construct; identical to SEQ ID NO: 89
  source 1..1392: mol_type = other DNA

SEQ ID NO: 126 - DNA, 609 bp, synthetic construct; identical to SEQ ID NO: 84
  source 1..609: mol_type = other DNA

SEQ ID NO: 127 - DNA, 2370 bp, synthetic construct
  source 1..2370: mol_type = other DNA
caaatgcagctgcaggagagcggtcctggcctggtgaagcctggcgagacactgagcctgacatgcagcgtgtccggcgccagcatcagcagcagctactggagctggctgcgggaaacccctggaaaaggcctggaatggatcggctacacccaccacagcggcgacacaaattacgccccaagcctcaagtccagagtgcacctgggcctgcaccccagcaagaaccaggtgtccctgtctctgaccagcgtcaccgcagccgacaccgctgtgtactactgcgccagaaccctgcatggcagaaggatctacggcgttgtggcctttaacgagttcttcacctacttctattgggaggtgtggggcaaaggcacccaggtgacagtgtctagcggcggagtgggcggcgagggcggcggtggctccggcagtgacatcagcgtggctcccggcgaaacagtgcggatctcttgtggcggagaaagcattggatctagagccgtgcagtggtaccagcacagagccggccaggccccaaagctgatcatctacaacaaccaggatagacctcctggcatccccgagcggttcagcggcagccctgacatcgacttcggcaccaccgccaccctgaccatcaccaatgtggaagccggcgatgaggccacatactattgccacatctgggactccagacggcccaccaactgggtctttggcggaggaacaaccctgacagtgctgggccaaggcggaagcggcgggtccggcggctctggaggcagcggcggctctggcggaagcggaggctctggcggcagcggcggcagcggcggcgacaagacccacacctgtcccccttgccccgctcccgagctgctgggaggcccctccgtgttcctgttccccccaaagcccaaagataccctgatgatcagcaggaccccggaggtgacctgcgtggtggtggacgtgagccacgaggaccctgaggtgaagttcaactggtacgtggacggcgtggaggttcacaacgccaagaccaaacccagggaggagcagtacaacagcacttacagggtggtgagcgtgctgaccgtgctgcaccaggactggctgaacggcaaggagtacaaatgcaaggttagcaacaaggcccttccagcccccatcgagaagaccatcagcaaggccaagggacagcctagggagccccaggtctacactctgccccccagccgcgacgagctgaccaagaaccaggtgagtttgtggtgtctggtgaagggcttctaccccagcgacatcgccgtggagtgggagagcaacggccagcccgagaacaactacaaaaccaccccccctgtgctggactcagacggcagctttttcctgtattctaagctcaccgtggacaagagcaggtggcagcagggcaacgtgttcagttgcagcgtgatgcacgaggccctgcacaaccactacacccagaagagcttgagcctgagccccggaaagggcggcggtggctccggcggcgggggcagcggcggaggcggaagcgacatcgtgctgacccagtctccactgagcctgtctgtgactcctggcgagtccgccagcatcagctgcaagagcagccagagcctgatccacggcgatagaaacaactacctggcctggtacgtgcagaagcccggcagaagccctcagctgctgatcgatctggcttctgagagagcctctggagtgcccgaccggtttagcggcagcggatctagaaaggacttcaccctgaagatcagcagagtggaaaccgaggatgtgggcacctattactgtatgcagggcagggaaagcccttggaccttcggccagggcaccaaggtggatattaagggcggcggtggctccggcggcgggggcagcggcggaggcggaagccaggcccagctggtgcagagcggacctgaggtgcggaagcctggcacaagcgtcaaggtgagctgcaaaaaccccggcaacacactgacaacatacgacctgcactgggtgcggcaggtgcctggccaaggactgcagtggatgggctggatcagccacgagggcgacaagaaagtcatcgtggaaagattcaaggccaaggtgacaatcgactgggacagaagcacaaacaccgcctacctgcaactgagcggcctgaccagcgaggataccgctgtgtattactgcgccaaaggctctaagcatagactgcgggactacgccctgtacgacgaggacggcgccctgaattgggccgtggacgttgattacctcgctaatctggagttctggggccagggcaccgccgtgaccgtgagcagc

SEQ ID NO: 128 - DNA, 1392 bp, synthetic construct; identical to SEQ ID NO: 89
  source 1..1392: mol_type = other DNA

SEQ ID NO: 129 - DNA, 609 bp, synthetic construct; identical to SEQ ID NO: 84
  source 1..609: mol_type = other DNA

SEQ ID NO: 130 - DNA, 1521 bp, synthetic construct; identical to SEQ ID NO: 82
  source 1..1521: mol_type = other DNA

SEQ ID NO: 131 - DNA, 2241 bp, synthetic construct
  source 1..2241: mol_type = other DNA
gctgatctggtgcagtctggcgccgtggtgaagaaacctggcgacagcgtgcggatttcttgtgaagcccagggttatagattccccgactacatcatccactggatcagaagagcccctggacagggacctgagtggatgggctggatgaaccccatgggcggccaagtgaacatcccttggaagttccagggcagagtgtccatgaccagacagctgagccaggacccagatgaccctgattggggcaccgcttttctggacctgagaggcctgaagtccgacgacaccgccgtctactactgcgtgcgggacagaagcaacggcagcggcaagcggttcgagagcagcaattggttcctggatctgtggggaaggggcacagccgttacaatccagagcgccagcaccaagggtcccagcgtattccccctggccccaagtagcaaaagcaccagtggtggcacagcggccctcggatgcctggttaaggactatttccccgagccggtaaccgtaagctggaacagtggagcactgaccagcggcgtgcataccttccccgctgtactgcaatcctccggcctgtactcactgagcagcgtagtgacggtgccctctagcagcctgggcacccagacctacatctgcaacgtgaaccataaaccgtctaacacgaaggtcgacaaaaaagttgagcctaaaagctgcgacaagacccacacctgtcccccttgccccgctcccgagctgctgggaggcccctccgtgttcctgttccccccaaagcccaaagataccctgatgatcagcaggaccccggaggtgacctgcgtggtggtggacgtgagccacgaggaccctgaggtgaagttcaactggtacgtggacggcgtggaggttcacaacgccaagaccaaacccagggaggagcagtacaacagcacttacagggtggtgagcgtgctgaccgtgctgcaccaggactggctgaacggcaaggagtacaaatgcaaggttagcaacaaggcccttccagcccccatcgagaagaccatcagcaaggccaagggacagcctagggagccccaggtctacactctgccccccagccgcgacgagctgaccaagaaccaggtgagtttgtcttgtgctgtgaagggcttctaccccagcgacatcgccgtggagtgggagagcaacggccagcccgagaacaactacaaaaccaccccccctgtgctggactcagacggcagctttttcctggtctctaagctcaccgtggacaagagcaggtggcagcagggcaacgtgttcagttgcagcgtgatgcacgaggccctgcacaaccactacacccagaagagcttgagcctgagccccggaaagggcggcggtggctccggcggcgggggcagcggcggaggcggaagccaggcccagctggtgcagagcggacctgaggtgcggaagcctggcacaagcgtcaaggtgagctgcaaaaaccccggcaacacactgacaacatacgacctgcactgggtgcggcaggtgcctggccaaggactgcagtggatgggctggatcagccacgagggcgacaagaaagtcatcgtggaaagattcaaggccaaggtgacaatcgactgggacagaagcacaaacaccgcctacctgcaactgagcggcctgaccagcgaggataccgctgtgtattactgcgccaaaggctctaagcatagactgcgggactacgccctgtacgacgaggacggcgccctgaattgggccgtggacgttgattacctcgctaatctggagttctggggccagggcaccgccgtgaccgtgagcagcggcggcggtggctccggcggcgggggcagcggcggaggcggaagcgacatcgtgctgacccagtctccactgagcctgtctgtgactcctggcgagtccgccagcatcagctgcaagagcagccagagcctgatccacggcgatagaaacaactacctggcctggtacgtgcagaagcccggcagaagccctcagctgctgatcgatctggcttctgagagagcctctggagtgcccgaccggtttagcggcagcggatctagaaaggacttcaccctgaagatcagcagagtggaaaccgaggatgtgggcacctattactgtatgcagggcagggaaagcccttggaccttcggccagggcaccaaggtggatattaag

SEQ ID NO: 132 - DNA, 609 bp, synthetic construct; identical to SEQ ID NO: 84
  source 1..609: mol_type = other DNA

SEQ ID NO: 133 - DNA, 1521 bp, synthetic construct; identical to SEQ ID NO: 82
  source 1..1521: mol_type = other DNA

SEQ ID NO: 134 - DNA, 2241 bp, synthetic construct
  source 1..2241: mol_type = other DNA
gctgatctggtgcagtctggcgccgtggtgaagaaacctggcgacagcgtgcggatttcttgtgaagcccagggttatagattccccgactacatcatccactggatcagaagagcccctggacagggacctgagtggatgggctggatgaaccccatgggcggccaagtgaacatcccttggaagttccagggcagagtgtccatgaccagacagctgagccaggacccagatgaccctgattggggcaccgcttttctggacctgagaggcctgaagtccgacgacaccgccgtctactactgcgtgcgggacagaagcaacggcagcggcaagcggttcgagagcagcaattggttcctggatctgtggggaaggggcacagccgttacaatccagagcgccagcaccaagggtcccagcgtattccccctggccccaagtagcaaaagcaccagtggtggcacagcggccctcggatgcctggttaaggactatttccccgagccggtaaccgtaagctggaacagtggagcactgaccagcggcgtgcataccttccccgctgtactgcaatcctccggcctgtactcactgagcagcgtagtgacggtgccctctagcagcctgggcacccagacctacatctgcaacgtgaaccataaaccgtctaacacgaaggtcgacaaaaaagttgagcctaaaagctgcgacaagacccacacctgtcccccttgccccgctcccgagctgctgggaggcccctccgtgttcctgttccccccaaagcccaaagataccctgatgatcagcaggaccccggaggtgacctgcgtggtggtggacgtgagccacgaggaccctgaggtgaagttcaactggtacgtggacggcgtggaggttcacaacgccaagaccaaacccagggaggagcagtacaacagcacttacagggtggtgagcgtgctgaccgtgctgcaccaggactggctgaacggcaaggagtacaaatgcaaggttagcaacaaggcccttccagcccccatcgagaagaccatcagcaaggccaagggacagcctagggagccccaggtctacactctgccccccagccgcgacgagctgaccaagaaccaggtgagtttgtcttgtgctgtgaagggcttctaccccagcgacatcgccgtggagtgggagagcaacggccagcccgagaacaactacaaaaccaccccccctgtgctggactcagacggcagctttttcctggtctctaagctcaccgtggacaagagcaggtggcagcagggcaacgtgttcagttgcagcgtgatgcacgaggccctgcacaaccactacacccagaagagcttgagcctgagccccggaaagggcggcggtggctccggcggcgggggcagcggcggaggcggaagcgacatcgtgctgacccagtctccactgagcctgtctgtgactcctggcgagtccgccagcatcagctgcaagagcagccagagcctgatccacggcgatagaaacaactacctggcctggtacgtgcagaagcccggcagaagccctcagctgctgatcgatctggcttctgagagagcctctggagtgcccgaccggtttagcggcagcggatctagaaaggacttcaccctgaagatcagcagagtggaaaccgaggatgtgggcacctattactgtatgcagggcagggaaagcccttggaccttcggccagggcaccaaggtggatattaagggcggcggtggctccggcggcgggggcagcggcggaggcggaagccaggcccagctggtgcagagcggacctgaggtgcggaagcctggcacaagcgtcaaggtgagctgcaaaaaccccggcaacacactgacaacatacgacctgcactgggtgcggcaggtgcctggccaaggactgcagtggatgggctggatcagccacgagggcgacaagaaagtcatcgtggaaagattcaaggccaaggtgacaatcgactgggacagaagcacaaacaccgcctacctgcaactgagcggcctgaccagcgaggataccgctgtgtattactgcgccaaaggctctaagcatagactgcgggactacgccctgtacgacgaggacggcgccctgaattgggccgtggacgttgattacctcgctaatctggagttctggggccagggcaccgccgtgaccgtgagcagc

SEQ ID NO: 135 - DNA, 609 bp, synthetic construct; identical to SEQ ID NO: 84
  source 1..609: mol_type = other DNA

SEQ ID NO: 136 - DNA, 57 bp, synthetic construct
  source 1..57: mol_type = other DNA
atggactggacatggcggatcctgtttctggtggctgctgccacaggtgcccattct

SEQ ID NO: 137 - DNA, 60 bp, synthetic construct
  source 1..60: mol_type = other DNA
atggaaaccgatacactgctgctgtgggtgctgttgctctgggtgccaggatctacaggc

SEQ ID NO: 138 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 139 - protein, 17 aa, synthetic construct
GQGGGGSGGGGSGGGGS

SEQ ID NO: 140 - protein, 24 aa, synthetic construct
GGGGSGGGGSGGGGSGGGGSGGGG

SEQ ID NO: 141 - protein, 5 aa, synthetic construct
GGGGS

SEQ ID NO: 142 - protein, 493 aa, synthetic construct
ALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQGGGGSGGGGSGGGGSADLVQSGAVVKKPGDSVRISCEAQGYRFPDYIIHWIRRAPGQGPEWMGWMNPMGGQVNIPWKFQGRVSMTRQLSQDPDDPDWGTAFLDLRGLKSDDTAVYYCVRDRSNGSGKRFESSNWFLDLWGRGTAVTIQSGGGGSGGGGSGGGGSGGGGSGGGGSGGGGSGGGGSDISVAPGETVRITCGGESIGSRAVQWYQHRPGQAPRLIIYNNQDRPPGIPERFSGSPDIDFGTTATLTISNVEAGDEATYYCHIWDSRRPTNWELGPGTTLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 143 - protein, 34 aa, synthetic construct
GGGGSGGGGSGGGGSGGGGSGGGGSGGGGSGGGG